(19) United States
(12) Patent Application Publication
(10) Pub. No.: US 2003/0175294 A1
(43) Pub. Date: Sep. 18, 2003

(54) Heterologous fusion protein constructs comprising a Leishmania antigen

(75) Inventors: Yasir Skeiky, Bellevue, WA (US); Mark Brannon, Seattle, WA (US); Jeffrey Guderian, Lynwood, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Corixa Corporation, Seattle, WA (US)

(21) Appl. No.: 10/098,732

(22) Filed: Mar. 13, 2002

Related U.S. Application Data
(60) Provisional application No. 60/275,837, filed on Mar. 13, 2001.

(51) Int. Cl. A61K 39/02; A61K 39/002; C07H 21/04; C12N 9/02; C12P 21/02; C12N 1/21; C12N 15/74

(52) U.S. Cl. 424/190.1; 424/191.1; 435/69.3; 435/252.3; 435/189; 435/320.1; 536/23.7

(57) ABSTRACT

The present invention provides a recombinant nucleic acid molecule encoding a fusion polypeptide, wherein the recombinant nucleic acid comprises a heterologous polynucleotide sequence encoding an antigen or an antigenic fragment, and a Leishmania polynucleotide sequence encoding a polypeptide or fragment thereof, wherein the Leishmania polynucleotide is selected from the group consisting of TSA polynucleotide, LeIF polynucleotide, M15 polynucleotide, and 6H polynucleotide. The invention also provides an expression cassette comprising the recombinant nucleic acid molecule, host cells comprising the expression cassette, compositions, fusion polypeptides, and methods of their use in diagnosis or in generating a protective immune response in hosts.

### CROSS-REFERENCES TO RELATED APPLICATIONS

The present application claims priority to U.S. Ser. No. 60/275,837, filed Mar. 13, 2001, herein incorporated by reference in its entirety.

The present application is related to U.S. patent application Ser. No. 09/056,556, filed Apr. 7, 1998; U.S. patent application Ser. No. 09/223,040, filed Dec. 30, 1998; U.S. patent application Ser. No. 09/287,849, filed Apr. 7, 1999; published PCT application No. WO 99/51748, filed Apr. 7, 1999 (PCT/US99/07717); U.S. patent application Ser. No. 60/158,338, filed Oct. 7, 1999; U.S. patent application Ser. No. 60/158,425, filed Oct. 7, 1999; U.S. patent application Ser. No. 09/597,796, filed Jun. 20, 2000; U.S. patent application Ser. No. 09/688,672, filed Oct. 10, 2000; published PCT application No. WO 01/24820, filed Oct. 10, 2000 (PCT/US00/28095); U.S. patent application Ser. No. 60/265,737, filed Feb. 1, 2001; U.S. patent application Ser. No. 09/886,349, filed Jun. 20, 2001; and published PCT application No. WO 01/98460, filed Jun. 20, 2001 (PCT/US01 19959), and U.S. Ser. No. 60/______, filed Feb. 15, 2002, TTC attorney docket number 014058-009080US, herein each incorporated by reference in its entirety.

### STATEMENT AS TO RIGHTS TO INVENTIONS MADE UNDER FEDERALLY SPONSORED RESEARCH AND DEVELOPMENT

Not applicable.

### FIELD OF THE INVENTION

This present invention relates to recombinant nucleic acids containing Leishmania TSA, LeIF, M15 or 6H polynucleotide encoding a polypeptide or a fragment thereof and a heterologous polynucleotide encoding an antigen or an antigenic fragment, such as Mycobacterium sp. antigens. In particular, it relates to using these nucleic acids as DNA vaccines to elicit protective immunity against pathogenic microorganisms in the host. The present invention also relates to expression cassettes comprising the recombinant nucleic acids, host cells comprising the expression cassettes, compositions, fusion polypeptides, and methods of their use in diagnosis or in generating a protective immune response in hosts.

### BACKGROUND OF THE INVENTION

Tuberculosis is a chronic infectious disease caused by infection with M. tuberculosis and other Mycobacterium species. It is a major disease in developing countries, as well as an increasing problem in developed areas of the world, with about 8 million new cases and 3 million deaths each year. Although the infection may be asymptomatic for a considerable period of time, the disease is most commonly manifested as an acute inflammation of the lungs, resulting in fever and a nonproductive cough. If untreated, serious complications and death typically result.

Although tuberculosis can generally be controlled using extended antibiotic therapy, such treatment is not sufficient to prevent the spread of the disease. Infected individuals may be asymptomatic, but contagious, for some time. In addition, although compliance with the treatment regimen is critical, patient behavior is difficult to monitor. Some patients do not complete the course of treatment, which can lead to ineffective treatment and the development of drug resistance.

In order to control the spread of tuberculosis, effective vaccination and accurate early diagnosis of the disease are of utmost importance. Currently, vaccination with live bacteria is the most efficient method for inducing protective immunity. The most common Mycobacterium employed for this purpose is Bacillus Calmette-Guerin (BCG), an avirulent strain of M. bovis. However, the safety and efficacy of BCG is a source of controversy and some countries, such as the United States, do not vaccinate the general public with this agent.

Diagnosis of tuberculosis is commonly achieved using a skin test, which involves intradermal exposure to tuberculin PPD (protein-purified derivative). Antigen-specific T cell responses result in measurable induration at the injection site by 48-72 hours after injection, which indicates exposure to Mycobacterium antigens. Sensitivity and specificity have, however, been a problem with this test, and individuals vaccinated with BCG cannot be distinguished from infected individuals.

While macrophages have been shown to act as the principal effectors of Mycobacterium immunity, T cells are the predominant inducers of such immunity. The essential role of T cells in protection against Mycobacterium infection is illustrated by the frequent occurrence of Mycobacterium infection in AIDS patients, due to the depletion of CD4⁺ T cells associated with human immunodeficiency virus (HIV) infection. Mycobacterium-reactive CD4⁺ T cells have been shown to be potent producers of γ-interferon (IFN-γ), which, in turn, has been shown to trigger the anti-mycobacterial effects of macrophages in mice. While the role of IFN-γ in humans is less clear, studies have shown that 1,25-dihydroxy-vitamin D3, either alone or in combination with IFN-γ or tumor necrosis factor-alpha, activates human macrophages to inhibit M. tuberculosis infection. Furthermore, it is known that IFN-γ stimulates human macrophages to make 1,25-dihydroxy-vitamin D3. Similarly, interleukin-12 (IL-12) has been shown to play a role in stimulating resistance to M tuberculosis infection. For a review of the immunology of M. tuberculosis infection, see Chan & Kaufmann, Tuberculosis: Pathogenesis, Protection and Control (Bloom ed., 1994), and Harrison's Principles of Internal Medicine, volume 1, pp. 1004-1014 and 1019-1023 (14^{th} ed., Fauciet al., eds., 1998).

Accordingly, there is a need for improved diagnostic reagents, and improved methods for diagnosis, preventing and treating tuberculosis. Embodiments of the invention meet this and other goals.

### SUMMARY OF THE INVENTION

The present invention is based, in part, on the discovery that when a heterologous polynucleotide sequence is fused to a Leishmania thiol-specific thiol-specific-antioxidant (herein referred to as “TSA” or “MAPS”), the Leishmania polynucleotide increases the expression of heterologous polynucleotide in eukaryotic cells. In addition to Leishmania TSA polynucleotide, embodiments of the invention provide that other Leishmania polynucleotides that expresses at a high level in eukaryotic cells, such as LeIF (a L. braziliensis gene homologous to the eukaryotic ribosomal protein eIF4A, also referred to as “LbeIF4A”), M15 (L. major stress-inducible 1 or LmSTII) or 6H (L. braziliensis gene homologous to the gene for the eukaryotic 83-kDa heat shock protein, also referred to as “Lbhsp83”), can also be used to make fusion constructs of the invention. Embodiments of the invention also provide that by optimizing the codons of the heterologous polynucleotides for maximal expression in eukaryotic cells, the expression of the fusion constructs can be further enhanced in eukaryotic cells. The Leishmania antigen can be at the N- or C-terminal region of the fusion protein, or may be found at any position in a fusion protein that comprises more than two antigens.

Any suitable heterologous polynucleotides that encode an antigen or an antigenic fragment can be fused to the Leishmania TSA, LeIF, M15 or 6H sequences. Typically, the heterologous polynucleotide is selected from those that encode a viral antigen such as HIV, HSV, CMV, or an Ebola antigen, a malaria antigen, a cancer antigen, or a bacterial antigen. In a preferred embodiment, a heterologous polynucleotide is a Mycobacterium polynucleotide sequence encoding an antigen or antigenic fragment thereof from a Mycobacterium species of the tuberculosis complex. In another preferred embodiment, the antigen is a Mycobacterium fusion protein, e.g., MTB72F±85b antigen. In another embodiment, the fusion protein comprises an RA35 antigen (full length, mature, or N-terminal portion of mature or full length Ra35) with a serine to alanine mutation at the triad active site at amino acid position 183 in wild-type MTB32A (Ra35).

The present fusion constructs are useful for enhancing the expression of Mycobacterium polynucleotides, as well as other heterologous polynucleotides which otherwise express poorly in eukaryotic cells. Moreover, the present invention constructs are particularly useful, among others, as DNA vaccines against, e.g., infections by one or more pathogenic microorganisms.

The present invention is also based, in part, on the discovery that when a heterologous polynucleotide is fused to a Leishmania TSA polynucleotide, the Leishmania polynucleotide fusion polypeptide elicits a strong cellular immune response when administered to a mammal. Thus, the present fusion constructs are useful, among others, in eliminating altered self-cells (e.g., virus-infected cells and tumor cells) in the host.

Accordingly, in one aspect, the invention provides a recombinant nucleic acid molecule encoding a fusion polypeptide, wherein the recombinant nucleic acid comprises a heterologous polynucleotide encoding an antigen or an antigenic fragment, and a Leishmania polynucleotide sequence encoding a polypeptide or a fragment thereof, wherein the Leishmania polynucleotide is selected from the group consisting of TSA polynucleotide, LeIF polynucleotide, M15 polynucleotide, and 6H polynucleotide. The invention also provides an expression cassette comprising the recombinant nucleic acid molecule, host cells comprising the expression cassette, and compositions comprising the expression cassette, and fusion polypeptides.

In one embodiment, the fusion polynucleotide and polypeptide comprise a relatively short fragment of a gene or a polypeptide, respectively, derived from the Leishmania TSA, LeIF, M15 or 6H so that a minimal immune response is elicited against the Leishmania polypeptide fragment in the host. In another embodiment, the heterologous polynucleotide is a Mycobacterium polynucleotide, preferably those that encode for MTB8.4 antigen, MTB9.8 antigen, MTB9.9 antigen, MTB12 antigen, MTB32A antigen, MTB40 antigen, MTB41 antigen, TbH9 antigen, Ra35 antigen, Ra12 antigen, 38-1 antigen, TbRa3 antigen, 38 kD antigen, DPEP antigen, TbH4 antigen, DPPD antigen, MTB82 antigen, Erd14 antigen, ESAT-6 antigen, MTB85 complex antigen, or an immunogenic fragment thereof. In another embodiment, Mycobacterium polynucleotide encodes for fusion proteins with two or more Mycobacterium antigens, such as MTB59F antigen, MTB72F antigen, MTB3 1F antigen, MTB71F antigen, or an immunogenic fragment thereof. In another embodiment, the Mycobacterium polynucleotide is codon optimized for expression in eukaryotic cells.

In another aspect, recombinant nucleic acid molecules, expression cassettes, compositions and fusion polypeptides may be used as immunogens to generate or elicit a protective immune response in a patient. The polynucleotides may be administered directly into a subject as DNA vaccines to cause antigen expression in the subject, and the subsequent induction of, e.g., an anti-M. tuberculosis immune response. Alternatively, the isolated or purified polynucleotides are used to produce recombinant fusion polypeptide antigens in vitro, which are then administered as a vaccine. Thus, the isolated or purified fusion Leishmania polypeptides and nucleic acids of the invention may be formulated as pharmaceutical compositions for administration into a subject in the prevention or treatment of Leishmania infections and/or infections by other microorganisms, such as M. tuberculosis. The immunogenicity of the fusion protein or antigens may be enhanced by the inclusion of an adjuvant, as well as additional fusion polypeptides, from Mycobacterium or other organisms, such as bacterial, viral, mammalian polypeptides. Additional polypeptides may also be included in the compositions, either linked or unlinked to the fusion polypeptide or compositions.

In another aspect, recombinant nucleic acid molecules, expression cassettes, compositions and fusion polypeptides of the invention are used in in vitro and in vivo assays for detecting humoral antibodies or cell-mediated immunity against one or more pathogenic microorganisms (e.g., M. tuberculosis and/or Leishmania) for diagnosis of infection or monitoring of disease progression. For example, the polypeptides may be used as an in vivo diagnostic agent in the form of an intradermal skin test. The polypeptides may also be used in in vitro tests such as an ELISA with patient serum. Alternatively, the nucleic acids, the compositions, and the fusion polypeptides may be used to raise, e.g., anti-M. tuberculosis antibodies in a non-human animal. The antibodies can be used to detect the target antigens in vivo and in vitro.

### DEFINITIONS

“Leishmania polynucleotide” that encodes a polypeptide or a fragment thereof refers to a native Leishmania polynucleotide found in Leishmania cells, fragments thereof, or any conservatively modified variants thereof. Functionally, a Leishmania polynucleotide has the ability to produce a fusion protein, and enhances expression relative to expression of a native full length Mycobacterium polynucleotide or portion thereof, or fusion thereof (e.g., MTB8.4, MTB12, MTB72F, 85b complex antigen, MTB72F plus 85b complex antigen (MTB103F), TB38-1 antigen, etc.) by at least 10%, optionally at least by 20%, 30%, 40%, 50%, 100%, or 200%.

“Fusion polypeptide” or “fusion protein” refers to a protein having at least two heterologous polypeptides covalently linked, either directly or via an amino acid linker. The polypeptides forming the fusion protein are typically linked C-terminus to N-terminus, although they can also be linked C-terminus to C-terminus, N-terminus to N-terminus, or N-terminus to C-terminus. The polypeptides of the fusion protein can be in any order. This term also refers to conservatively modified variants, polymorphic variants, alleles, mutants, subsequences, and interspecies homologs of the antigens that make up the fusion protein. In embodiments of the invention, typically a Leishmania polypeptide or a fragment thereof is fused to a heterologous polypeptide, such as Mycobacterium tuberculosis antigen or a fragment thereof. The Leishmania antigen can be fused to the Mycobacterium tuberculosis antigen (or other heterologous antigen) at either the N-or C-terminus, or for a fusion protein of more than two members, at any position. Mycobacterium tuberculosis antigens are described in Cole et al., Nature 393:537 (1998), which discloses the entire Mycobacterium tuberculosis genome. The complete sequence of Mycobacterium tuberculosis can also be found at http://www.sanger.ac.uk and at http://www.pasteur.fr/mycdb/ (MycDB). Antigens from other Mycobacterium species that correspond to M. tuberculosis antigens can be identified, e.g., using sequence comparison algorithms, as described herein, or other methods known to those of skill in the art, e.g., hybridization assays and antibody binding assays.

Typically, a fusion polypeptide of the invention specifically binds to antibodies raised against at least two antigen polypeptides, wherein each antigen polypeptide is selected from the group consisting of a Leishmania TSA, LeIF, M15 or 6H polypeptide and a heterologous polypeptide, such as a Mycobacterium polypeptide. The antibodies can be polyclonal or monoclonal. Optionally, the fusion polypeptide specifically binds to antibodies raised against the fusion junction of the antigens, which antibodies do not bind to the antigens individually, i.e., when they are not part of a fusion protein. The fusion polypeptides optionally comprise additional polypeptides, e.g., three, four, five, six, or more polypeptides, up to about 25 polypeptides, optionally heterologous polypeptides or repeated homologous polypeptides, fused to the at least two heterologous antigens. The additional polypeptides of the fusion protein are optionally derived from Mycobacterium as well as other sources, such as other bacterial, viral, or invertebrate, vertebrate, or mammalian sources. The individual polypeptides of the fusion protein can be in any order. As described herein, the fusion protein can also be linked to other molecules, including additional polypeptides. The compositions of the invention can also comprise additional polypeptides that are unlinked to the fusion proteins of the invention. These additional polypeptides may be heterologous or homologous polypeptides.

The term “fused” refers to the covalent linkage between two polypeptides in a fusion protein. The polypeptides are typically joined via a peptide bond, either directly to each other or via an amino acid linker. Optionally, the peptides can be joined via non-peptide covalent linkages known to those of skill in the art.

“FL” refers to full-length, i.e., a polypeptide that is the same length as the wild-type polypeptide.

In some instances in the application, Ra35 refers to the N-terminus of MTB32A (Ra35FL), comprising at least about the first 205 amino acids of MTB32A from M. tuberculosis, or the corresponding region from another Mycobacterium species. Ra12 refers to the C-terminus of MTB32A (Ra35FL), comprising at least about the last 132 amino acids from MTB32A from M. tuberculosis, or the corresponding region from another Mycobacterium species.

The following provides sequences of some individual antigens used in the compositions and fusion proteins of the invention:

MTB32A (TbRa35FL), the sequence of which is disclosed as SEQ ID NO:17 (cDNA) and SEQ ID NO:79 (protein) in the U.S. patent applications Ser. No. 08/523,436, 08/523,435, No. 08/658,800, No. 08/659,683, No. 08/818,112, No. 09/056,556, and No. 08/818,111 and in the WO97/09428 and WO97/09429 applications, see also Skeiky et al., Infection and Immunity 67:3998-4007 (1999);

MTBRa12, the C-terminus of MTB32A (Ra35FL), comprising at least about the last 132 amino acids from MTB32A from M. tuberculosis, the sequence of which is disclosed as SEQ ID NO:4 (DNA) and SEQ ID NO:66 (predicted amino acid sequence) in the U.S. patent application Ser. No. 09/072,967;

Ra35, the N-terminus of MTB32A (Ra35FL), comprising at least about the first 205 amino acids of MTB32A from M. tuberculosis, the nucleotide and amino acid sequence of which is disclosed in FIG. 4;

MTB39 (TbH9), the sequence of which is disclosed as SEQ ID NO:106 (cDNA full length) and SEQ ID NO:107 (protein full length) in the U.S. patent applications Ser. No. 08/658,800, No. 08/659,683, No. 08/818,112, and No. 08/818,111 and in the WO97/09428 and WO97/09429 applications. The sequence is also disclosed as SEQ ID NO:33 (DNA) and SEQ ID NO:91 (amino acid) in U.S. patent application Ser. No. 09/056,559;

The following provides sequences of some fusion proteins of the invention

TbH9-Ra35 (MTB59F), the sequence of which is disclosed as SEQ ID NO:23 (cDNA) and SEQ ID NO:24 (protein) in the U.S. patent application Ser. No. 09/287,849 and in the PCT/US99/07717 application;

RA12-TbH9-Ra35 (MTB72F), the sequence of which is disclosed as SEQ ID NO:1 (DNA) and SEQ ID NO:2 (protein) in the U.S. patent application Ser. No. 09/223,040, No. 09/223,040, and in the PCT/US99/07717 application.

RA12-TbH9-Ra35-85b antigen (MTB103F), the sequence of which is disclosed in U.S. Ser. No. 60/______, filed Feb. 15, 2002, TTC reference no. 014058-009080US.

The following provides sequences of some additional antigens used in the compositions and fusion proteins of the invention:

MTB8.4 (DPV), the sequence of which is disclosed as SEQ ID NO:101 (cDNA) and SEQ ID NO:102 (protein) in the U.S. patent applications Ser. No. 08/658,800, No. 08/659,683, No. 08/818,112 and No. 08/818,111 and in the WO97/09428 and WO97/09429 applications;

MTB9.8 (MSL), the sequence of which is disclosed as SEQ ID NO:12 (DNA), SEQ ID NO:109 (predicted amino acid sequence) and SEQ ID NO:110 to 124 (peptides) in the U.S. patent applications Ser. No. 08/859,381, No. 08/858,998, No. 09/073,009 and No. 09/073,010 and in the PCT/US98/10407 and PCT/US98/10514 applications;

MTB9.9A (MTI, also known as MTI-A), the sequence of which is disclosed as SEQ ID NO:3 and SEQ ID NO:4 (DNA) and SEQ ID NO:29 and SEQ ID NO:51 to 66 (ORF peptide for MTI) in the U.S. patent applications Ser. No. 08/859,381, No. 08/858,998, No. 09/073,009 and v09/073,010 and in the PCT/US98/10407 and PCT/US98/10514 applications. Two other MTI variants also exist, called MTI-B and MTI-C;

MTB40 (HTCC#1), the sequence of which is disclosed as SEQ ID NO:137 (cDNA) and 138 (predicted amino acid sequence) in the U.S. patent applications Ser. No. 09/073,009 and No. 09/073,010 and in the PCT/US98/10407 and PCT/US98/10514 applications;

MTB41 (MTCC#2), the sequence of which is disclosed as SEQ ID NO:140 (cDNA) and SEQ ID NO:142 (predicted amino acid sequence) in the U.S. patent applications Ser. No. 09/073,009 and No. 09/073,010 and in the PCT/US98/10407 and PCT/US98/10514 applications;

ESAT-6, the sequence of which is disclosed as SEQ ID NO:103 (DNA) and SEQ ID NO:104 (predicted amino acid sequence) in the U.S. patent application Ser. No. 09/072,967. The sequence of ESAT-6 is also disclosed in U.S. Pat. No. 5,955,077.

TB38-1, the sequence of which is disclosed as SEQ ID NO:46 (DNA) and SEQ ID NO:88 (amino acid) in U.S. Ser. No. 08/818,112 and U.S. Ser. No. 09/072,967.

α-crystalline antigen, the sequence of which is disclosed in Verbon et al., J. Bact. 174:1352-1359 (1992);

85 complex antigen, e.g., 85b complex antigen, the sequence of which is disclosed in Content et al., Infect. & Immunol. 59:3205-3212 (1991).

Each of the above sequences is also disclosed in Cole et al. Nature 393:537 (1998) and can be found at, e.g., http://www.sanger.ac.uk and http:/www.pasteur.fr/mycdb/.

The above sequences are disclosed in U.S. patent applications Ser. Nos. 08/523,435, 08/523,436, 08/658,800, 08/659,683, 08/818,111, 08/818,112, 08/942,341, 08/942,578, 08/858,998, 08/859,381, 09/056,556, 09/072,596, 09/072,967, 09/073,009, 09/073,010, 09/223,040, 09/287,849 and in PCT patent applications PCT/US98/10407, PCT/US98/10514, PCT/US99/03265, PCT/US99/03268, PCT/US99/07717, WO97/09428 and WO97/09429, WO98/16645, WO98/16646, each of which is herein incorporated by reference.

MTB32AMutSA is a mutated version of wild-type MTB32A (Ra35FL or Ra35 mature). The sequence of wild-type RA35 is disclosed as SEQ ID NO:17 (cDNA) and SEQ ID NO:79 (protein) in the U.S. patent applications Ser. No. 08/523,436, 08/523,435, No. 08/658,800, No. 08/659,683, No. 08/818,112, No. 09/056,556, and No. 08/818,111 and in the WO97/09428 and WO97/09429 applications, see also Skeiky et al., Infection and Immunity 67:3998-4007 (1999). The term mutated MTB32, mutated MTB32A, MTB32AMutSA or MTB32MutSA includes MTB32A amino acid sequences in which any one of the three amino acids at the active site triad (His, Asp, Ser, amino acid positions 182-184 of the wild type molecule), e.g., the serine residue at amino acid position 183 in wild-type MTB32A, has been changed to another amino acid (e.g., to alanine, Ra35FLMutSA, see, e.g., the sequence comparison of wild type and mutated MTB32 in FIG. 5).

The term “immunogenic fragment thereof” refers to a polypeptide comprising an epitope that is recognized by cytotoxic T lymphocytes, helper T lymphocytes or B cells.

The term “Mycobacterium species of the tuberculosis complex” includes those species traditionally considered as causing the disease tuberculosis, as well as Mycobacterium environmental and opportunistic species that cause tuberculosis and lung disease in immune compromised patients, such as patients with AIDS, e.g., M. tuberculosis, M. bovis, or M. africanum, BCG, M. avium, M. intracellulare, M. celatum, M. genavense, M. haemophilum, M. kansasii, M. simiae, M. vaccae, M. fortuitum, and M. scrofulaceum (see, e.g., Harrison's Principles of Internal Medicine, volume 1, pp. 1004-1014 and 1019-1023 (14^{th} ed., Fauci et al., eds., 1998).

An adjuvant refers to the components in a vaccine or therapeutic composition that increase the specific immune response to the antigen (see, e.g., Edelman, AIDS Res. Hum Retroviruses 8:1409-1411 (1992)). Adjuvants induce immune responses of the Th1-type and Th-2 type response. Th1-type cytokines (e.g., IFN-γ, IL-2, and IL-12) tend to favor the induction of cell-mediated immune response to an administered antigen, while Th-2 type cytokines (e.g., IL-4, IL-5, IL-6, IL-10 and TNF-β) tend to favor the induction of humoral immune responses.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

As used herein, the terms “DNA segment” and “polynucleotide” refer to a DNA molecule that has been isolated free of total genomic DNA of a particular species. Therefore, a DNA segment encoding a polypeptide refers to a DNA segment that contains one or more coding sequences yet is substantially isolated away from, or purified free from, total genomic DNA of the species from which the DNA segment is obtained. Included within the terms “DNA segment” and “polynucleotide” are DNA segments and smaller fragments of such segments, and also recombinant vectors, including, for example, plasmids, cosmids, phagemids, phage, viruses, and the like.

As will be understood by those skilled in the art, the DNA segments of this invention can include genomic sequences, extra-genomic and plasmid-encoded sequences and smaller engineered gene segments that express, or may be adapted to express, proteins, polypeptides, peptides and the like. Such segments may be naturally isolated, or modified synthetically by the hand of man.

The terms “isolated,” “purified,” or “biologically pure” therefore refer to material that is substantially or essentially free from components that normally accompany it as found in its native state. Of course, this refers to the DNA segment as originally isolated, and does not exclude other isolated proteins, genes, or coding regions later added to the composition by the hand of man. Purity and homogeneity are typically determined using analytical chemistry techniques such as polyacrylamide gel electrophoresis or high performance liquid chromatography. A protein that is the predominant species present in a preparation is substantially purified. An isolated nucleic acid is separated from other open reading frames that flank the gene and encode proteins other than the gene.

As will be recognized by the skilled artisan, polynucleotides may be single-stranded (coding or antisense) or double-stranded, and may be DNA (genomic, cDNA or synthetic) or RNA molecules. RNA molecules include mRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein, which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

(see, e.g., Creighton, Proteins (1984)).

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as a formamide. For selective or specific hybridization, a positive signal is at least two times background, optionally 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency.

An “expression cassette” refers to a polynucleotide molecule comprising expression control sequences operatively linked to coding sequence(s).

A “vector” is a replicon in which another polynucleotide segment is attached, so as to bring about the replication and/or expression of the attached segment.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of monoclonal or polyclonal antibodies, any technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy (1985)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to fusion proteins can be selected to obtain only those polyclonal antibodies that are specifically immunoreactive with fusion protein and not with individual components of the fusion proteins. This selection may be achieved by subtracting out antibodies that cross-react with the individual antigens. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

Polynucleotides may comprise a native sequence (i.e., an endogenous sequence that encodes an individual antigen or a portion thereof) or may comprise a variant of such a sequence. Polynucleotide variants may contain one or more substitutions, additions, deletions and/or insertions such that the biological activity of the encoded fusion polypeptide is not diminished, relative to a fusion polypeptide comprising native antigens. Variants preferably exhibit at least about 70% identity, more preferably at least about 80% identity and most preferably at least about 90% identity to a polynucleotide sequence that encodes a native polypeptide or a portion thereof.

The terms “identical” or percent “identity,” in the context of two or more nucleic acids or polypeptide sequences, refer to two or more sequences or subsequences that are the same or have a specified percentage of amino acid residues or nucleotides that are the same (i.e., 70% identity, optionally 75%, 80%, 85%, 90%, or 95% identity over a specified region), when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Optionally, the identity exists over a region that is at least about 25 to about 50 amino acids or nucleotides in length, or optionally over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 25 to 500, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Another example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. Software for performing BLAST analyses is publicly available through the National Center for Biotechnology Information (http://www.ncbi.nlm.nih.govl). This algorithm involves first identifying high scoring sequence pairs (HSPS) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

### BRIEF DESCRIPTION OF THE DRAWINGS

FIGS. 1A-C illustrate Western blots of DNA vaccine construct expression in HEK 293 cells. Panel A. Rabbit anti-DPV: MAPS-DPV-AC is strongly expressed in HEK cells, while all other DPV constructs are undetectable. Panel B. Rabbit anti-DPAS: While all DPAS constructs are expressed, fusion with MAPS results in increased expression. Panel C. Rabbit anti-MAPS: This panel demonstrates that, of the DPV constructs, only MAPS-DPV-AC is expressed and that MAPS-DPAS and MAPS-DPAS-AC are expressed at comparable levels. JA4304, negative control, shows no reactivity with any antibody.

FIG. 2 illustrates Western blots of DNA vaccine construct expression in HEK 293 cells. The left panel shows reactivity of fusion proteins with rabbit anti-DPV. The right panel shows reactivity of fusion proteins with rabbit anti-MAPS. Data indicate that fusion of the codon optimized DPV gene to sequences encoding the first give (MAPS(N5)/DPV-AC) and, in particular, the first ten (MAPS(N10)/DPV-AC) amino acids of MAPS significantly boosts the expression of these antigens in eukaryotic cells. The full length MAPS/DPV-AC are most highly expressed.

FIGS. 3A and 3B illustrate nucleotide and amino acid sequences of Leishmania thiol-specific-antioxidant (i.e., TSA or MAPS) having SEQ ID NOS: 66 and 67, respectively.

FIG. 4 illustrate nucleotide and amino acid sequences of Leishmania LeJF (i.e., LbeIF4A) having SEQ ID NOS: 68 and 69, respectively.

FIG. 5 illustrate nucleic acid and amino acid sequences of Leishmania M15 (i.e., LmSTI1) having SEQ ID NOS: 70 and 71, respectively.

FIG. 6 illustrate nucleic acid and amino acid sequences of Leishmania 6H (i.e., Lbhsp83) having SEQ ID NOS: 72 and 73, respectively.

### DETAILED DESCRIPTION

### Introduction

Vaccination with antigen encoding DNA constructs is an attractive alternative to protein-based vaccines. One potential problem for DNA vaccination, however, is that the level of antigen expression sufficient to elicit protective immunity is often not achieved. In some situations, this may be due to the fact that non-secreted, intracellular, heterologous proteins may not be highly expressed in eukaryotic cells. In other situations, this may be due to the fact that many organisms utilize codons differentially to obtain optimum protein expression. Therefore, a gene derived from an infectious disease agent, containing that microorganism's inherent codon bias, may not be expressed at a level high enough to provide protection in a mammalian model system of the disease. For example, low protein expression occurs for some Mycobacterium tuberculosis genes tested in DNA vaccination studies.

The present inventors discovered that the fusion to a gene known to express at high levels in eukaryotic cells can enhance the expression of heterologous polynucleotides in eukaryotic cells. For example, many Leishmania genes express at a high level in eukaryotic cells. These genes include, e.g., thiol-specific-antioxidant (herein referred to as “TSA” or “MAPS,” see, e.g., Webb et al., Infection and Immunity 66:3279-3289 (1998)), LeIF (also referred to as “LbeIF4A,” see, e.g., Skeiky et al., J. Exp. Med. 181:1527-1537 (1995); Skeiky et al., J. Immunol. 161:6171-6179 (1998)), M15 (also referred to as “LmSTI1,” see, e.g., Webb et al., J. Immunol. 157:5034-5041 (1996)), and 6H (also referred to as “Lbhsp83,” see, e.g., Skeiky et al., Infection and Immunity 63:4105-4114 (1995)). Preferably, these Leishmania sequences are fused at the N-terminus of the heterologous polynucleotide to enhance the efficiency of ribosome movement and hence the translation efficiency of the mRNA. In a preferred embodiment, TSA is used to produce a fusion construct. Moreover, the expression of a heterologous polynucleotide fused to these Leishmania polynucleotide can be further enhanced by optimizing the codon usage of the heterologous polynucleotide for maximal expression in eukaryotic cells. Therefore, the present fusion constructs are particularly useful as DNA vaccines to prevent, e.g., infections by pathogenic microorganisms.

Any heterologous sequences of interest can be fused to the Leishmania TSA, LeIF, M15 or 6H sequences. These include, but are not limited to, a Mycobacterium antigen, a HIV antigen, a HSV antigen, a CMV antigen, a malaria antigen, a cancer antigen, or other viral or bacterial antigens. Expression of these heterologous sequences can be enhanced by fusing them to the above described Leishmania sequences. Moreover, it has been found that the fusion of a heterologous sequence to a Leishmania sequence can elicit a strong cellular immune response in mammalian host. Thus, the present fusion constructs are useful, among others, in eliminating altered self-cells (e.g., virus-infected cells and tumor cells) in the host.

Accordingly, the present invention provides recombinant nucleic acid molecules encoding a fusion polypeptide, wherein the nucleic acid molecule comprises a heterologous polynucleotide sequence of interest and a Leishmania polynucleotide encoding a polypeptide or a fragment thereof, wherein the Leishmania polynucleotide is TSA polynucleotide, LeIF polynucleotide, M15 polynucleotide, or 6H polynucleotide. The invention also provides expression cassettes comprising the recombinant nucleic acid molecules, compositions comprising the expression cassettes, fusion polypeptides, and methods for their use.

Embodiments of the invention have many applications. For example, the present invention can be used to produce DNA vaccines against infections by microorganisms, such as Mycobacterium. In another example, by fusing polynucleotides that encode epitopes from two or more microorganisms, the present invention can be used as a vaccine against diseases caused by different infectious agents (e.g., Mycobacterium and Leishmania). In another example, embodiments of the invention can be used in vitro and in vivo assays for detecting humoral antibodies or cell-mediated immunity against Mycobacterium or other microorganisms for diagnosis of infection or monitoring of disease progression. Embodiments of the invention and their use are described in detail below.

### Recombinant Nucleic Acid Molecules

In one aspect, the invention provides recombinant nucleic acid molecules comprising a Leishmania TSA, LeIF, M15 or 6H polynucleotide sequence encoding a polypeptide or fragment thereof and a polynucleotide encoding an antigen or antigenic fragment of a microorganism, such as Mycobacterium. Recombinant nucleic acids are constructed so that, preferably, the Leishmania polynucleotide is located 5′ to a heterologous polynucleotide sequence of interest. It may also be appropriate to place a Leishmania polynucleotide 3′ to the heterologous polynucleotide sequence or to insert the heterologous polynucleotide sequence into a site within the Leishmania polynucleotide.

The recombinant nucleic acid molecules of the present invention, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative DNA segments with total lengths of about 10,000, about 5000, about 3000, about 2,000, about 1,000, about 500, about 200, about 100, about 50 base pairs in length, and the like, (including all intermediate lengths) are contemplated to be useful in many implementations of this invention. Preferably, the recombinant sequences are operably linked to a eukaryotic promoter, such as CMV, to provide expression cassettes.

1. Leishmania Polynucleotides

Any suitable Leishmania polynucleotides can be used for constructing recombinant fusion nucleic acid molecules of the present invention. For example, the Leishmania polynucleotides can be derived from TSA gene (see Webb et al., Infect. Immun. 66:3279-3289 (1998); GenBank Accession No. AF044679), LeIF gene (Skeiky et al., J. Exp. Med. 181:1527-1537 (1995); Skeiky et al., J. Immunol. 161:6171-6179 (1998)), M15 gene (Webb et al., J. Immunol. 157:5034-5041 (1996); GenBank Accession No. 473845), or 6H gene (Skeiky et al., J. Infec. Immun. 63:4105-4114 (1995)), all of the disclosures of which are incorporated herein by reference. The nucleic acid and amino acid sequences of LeIF, M15 and 6H are also described in U.S. Pat. No. 5,834,592, incorporated herein by reference. These genes express highly in eukaryotic cells, and any of these Leishmania genes can be used to make a fusion polynucleotide.

Typically, fusion to these Leishmania polynucleotides increase the expression of a heterologous polynucleotide fused to these Leishmania polynucleotides by at least 10%, optionally at least 20%, 30%, 40%, 50%, 100% or 200%, compared to the expression of the heterologous polynucleotide alone.

Either the full length Leishmania gene or a portion of the Leishmania gene can be included in fusion polynucleotides of the invention. For example, the Leishmania polynucleotides that encode a polypeptide or a fragment thereof can comprise at least about 15, 20, 30, 40, 50, 75, 100, 150, 200, 300, 400, 500 or 1000 or more contiguous nucleotides of one or more of the sequences disclosed herein as well as all intermediate lengths there between. It will be readily understood that “intermediate lengths,” in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like.

The selection of the length and portion of the Leishmania polynucleotide depends on whether an immune response against the Leishmania polypeptide is desired. If it is desired to elicit an immune response against a Leishmania polypeptide portion of the fusion construct, then the full length Leishmania gene or a portion that encodes a highly antigenic epitope is used. These constructs are capable of serving as an effective vaccine against at least two different infectious agent (Leishmania and another microorganism from which the fusion partner is derived).

If minimizing an immune response against a Leishmania polypeptide is desired, then preferably small fragments of a Leishmania gene are used. For example, a Leishmania polynucleotide included in the fusion construct may comprise about 90 nucleotides or less, about 60 nucleotides or less, about 30 nucleotides or less, about 15 nucleotides or less, or any intermediate lengths in between. Preferably, a Leishmania polynucleotide includes at least the 5′ portion of a Leishmania gene. As described in the example section, a Leishmania polynucleotide comprising the first 15 nucleotides or the first 30 nucleotides of the Leishmania TSA gene can enhance the expression of its fusion partner, without eliciting much immune response to TSA.

In embodiments of the invention, Leishmania polynucleotides may comprise a native sequence (i.e., an endogenous sequence that encodes TSA, LeIF, M15, 6H or a portion thereof) or may comprise a variant of such a sequence. Polynucleotide variants may contain one or more substitutions, additions, deletions and/or insertions such that the biological activity of the encoded fusion polypeptide is not diminished, relative to a fusion polypeptide comprising a native Leishmania polypeptide. Variants preferably exhibit at least about 70% identity, more preferably at least about 80% identity and most preferably at least about 90% identity to a polynucleotide sequence that encodes a native Leishmania polynucleotide or a portion thereof. Optionally, the identity exists over a region that is at least about 25 to about 50 nucleotides in length, or optionally over a region that is 75-100 nucleotides in length. Variants are preferably capable of hybridizing under stringent conditions to the native Leishmania sequences.

2. Fusion Partners to Leishmania Polynucleotides

In the present invention, any suitable heterologous polynucleotides of interest can be selected as a fusion partner to Leishmania polynucleotides. Typically, heterologous polynucleotides encode pathogenic antigens, bacterial antigens, viral antigens, cancer antigens, tumor antigens, tumor suppressors, or antigenic fragments thereof. In one embodiment, heterologous polynucleotides encode an antigen or antigenic fragment from a Mycobacterium species of the tuberculosis complex. In another embodiment, heterologous polynucleotides are derived from infectious agents, such as HIV, HSV, CMR, Ebola, or pathogenic agents that cause malaria (e.g., P. falciparum, P. vivax, P. malariae, and P. ovale).

Preferably, the fusion partner is derived from Mycobacterium polynucleotides encoding Mycobacterium antigens or fragments thereof can be coupled to a Leishmania polynucleotide. Mycobacterium polynucleotides are derived from a Mycobacterium species of the tuberculosis complex, e.g., a species such as M. tuberculosis, M. bovis, or M. africanum, or a Mycobacterium species that is environmental or opportunistic and that causes opportunistic infections such as lung infections in immune compromised hosts (e.g., patients with AIDS), e.g., BCG, M. avium, M. intracellulare, M. celatum, M. genavense, M. haemophilum, M. kansasii, M. simiae, M. vaccae, M. fortuitum, and M. scrofulaceum (see, e.g., Harrison's Principles of Internal Medicine, volume 1, pp. 1004-1014 and 1019-1023 (14^{th} ed., Fauci et al., eds., 1998).

In embodiments of the invention, Mycobacterium polynucleotides can encode a single antigen or immunogenic fragments thereof, or can encode at least two heterologous Mycobacterium antigens or immunogenic fragments thereof. Some fusion proteins comprising at least two heterologous Mycobacterium antigens, or immunogenic fragments thereof are sometimes highly antigenic. The antigens of the present invention may further comprise other components designed to enhance the antigenicity of the antigens or to improve these antigens in other aspects, for example, the isolation of these antigens through addition of a stretch of histidine residues at one end of the antigen.

Examples of Mycobacterium polynucleotides that can be fused to a Leishmania polynucleotide include those that encode Mycobacterium sp. antigens such as MTB8.4 antigen, MTB9.8 antigen, MTB9.9 antigen, MTB12 antigen, MTB32A antigen, MTB40 antigen, MTB41 antigen, TbH9 antigen, Ra35 antigen, Ra12 antigen, 38-1 antigen, TbRa3 antigen, 38 kD antigen, DPEP antigen, TbH4 antigen, DPPD antigen, MTB92 antigen, Erd14 antigen, ESAT-6 antigen, MTB85 complex antigen, MTB59F antigen, MTB72F antigen, MTB31F antigen, MTB71 antigen, or immunogenic fragment thereof.

The heterologous polynucleotide which is linked to a Leishmania polynucleotide encodes a polypeptide or a fragment comprising at least about 15, 20, 30, 40, 50, 75, 100, 150, 200, 300, 400, 500 or 1000 or more contiguous nucleotides of one or more of the sequences disclosed herein as well as all intermediate lengths there between. It will be readily understood that “intermediate lengths”, in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like.

In embodiments of the invention, heterologous polynucleotides may comprise a native sequence (e.g., an endogenous sequence from an organism's cells) or may comprise a conservatively modified variant of such a sequence or immunogenic fragment thereof. Polynucleotide variants may contain one or more substitutions, additions, deletions and/or insertions such that the biological activity of the encoded fusion polypeptide is not diminished, relative to a fusion polypeptide comprising a native heterologous polypeptide. Variants preferably exhibit at least about 70% identity, more preferably at least about 80% identity and most preferably at least about 90% identity to a polynucleotide sequence that encodes a native polynucleotide or a portion thereof. Optionally, the identity exists over a region that is at least about 25 to about 50 nucleotides in length, or optionally over a region that is 75-100 nucleotides in length. Variants are preferably capable of hybridizing under stringent conditions to the native sequences.

In some embodiments, the heterologous polynucleotides are optimized for eukaryotic codon selection, particularly for human and/or primate. As described above, most organisms exhibit differential codon usage for optimum protein expression. Thus, the expression of a Mycobacterium sp. genes in eukaryotic cells is often very poor. The expression of Mycobacterium or other heterologous polynucleotides can be enhanced by optimizing the codon usage of the polynucleotides for maximal expression in eukaryotic cells. Preferably, the codons are optimized for expression in mammals, particularly in human and/or in primates. The preferred codon usage in mammals and other vertebrates are described in, e.g., Current Protocols in Molecular Biology, vol. 4, Ausubel et al., ed., John Wiley & Sons, Inc., Appendix 1, incorporated herein by reference. Codon usage tables can also be found in www.kazusa.or.jp/codon/, incorporated herein by reference.

The following provides sequences of some Mycobacterium sp. antigens used in embodiments of the invention:

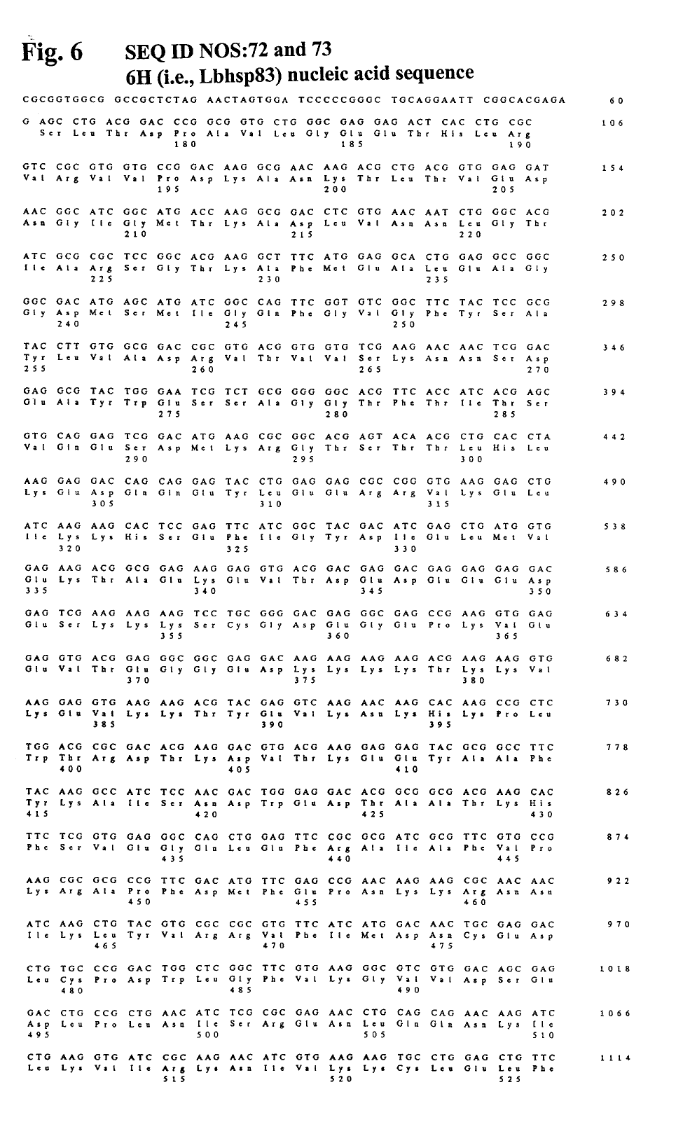

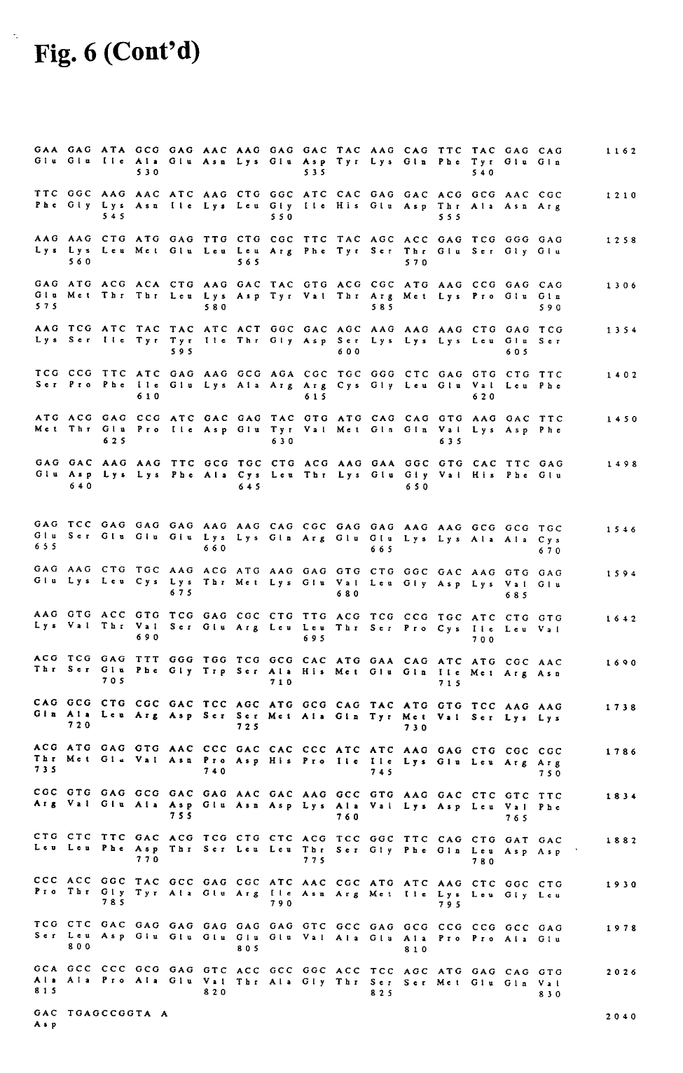

SEQ ID NO:1-4: MTB32A (Ra35FL or Ra35 mature), the sequence of which is also disclosed as SEQ ID NO:17 (cDNA) and SEQ ID NO:79 (protein) in the U.S. patent applications Ser. No. 08/523,436, 08/523,435, No. 08/658,800, No. 08/659,683, No. 08/818,112, No. 09/056,556, and No. 08/818,111 and in the WO97/09428 and WO97/09429 applications, see also Skeiky et al., Infection and Immunity 67:3998-4007 (1999). The term MTB32A also includes MTB32A amino acid sequences in which any one of the three amino acids at the active site triad (His, Asp, Ser), e.g., the serine residue at amino acid position 207 in SEQ ID NO:2 or amino acid position 183 in SEQ ID NO:4, has been changed to another amino acid (e.g., alanine, Ra35FLMutSA, see, e.g., FIG. 6 and SEQ ID NO:6).

SEQ ID NO:5 and 6: Ra35FLMut SA, the mature version of RA35FL in which the serine residue at amino acid position 183 of SEQ ID NO:4 has been changed to an alanine residue.

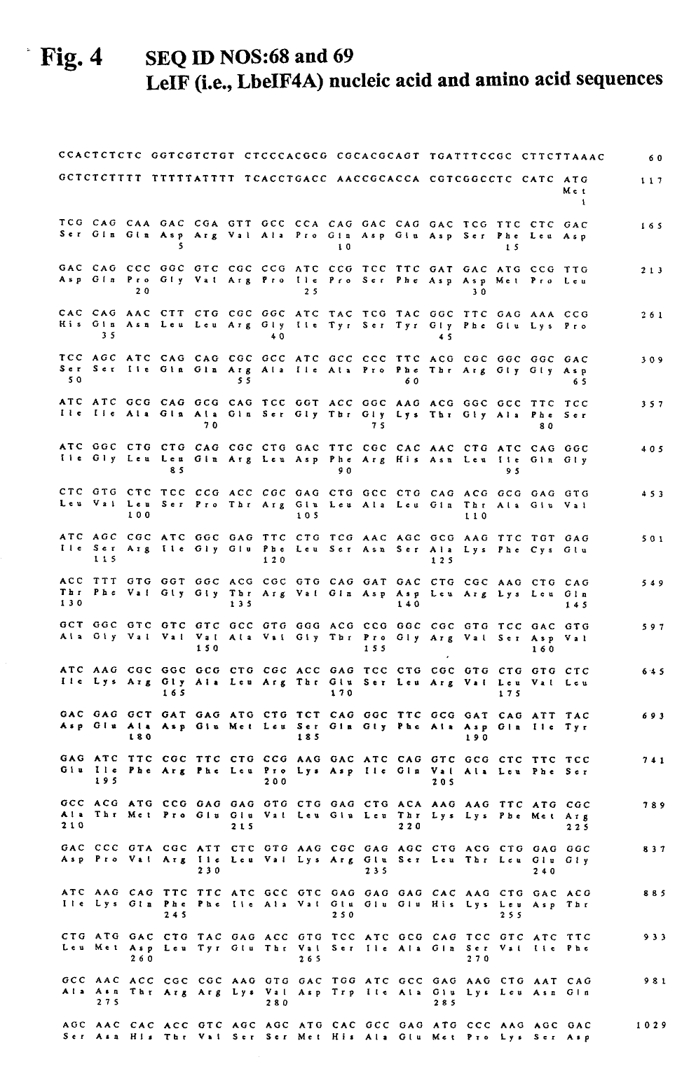

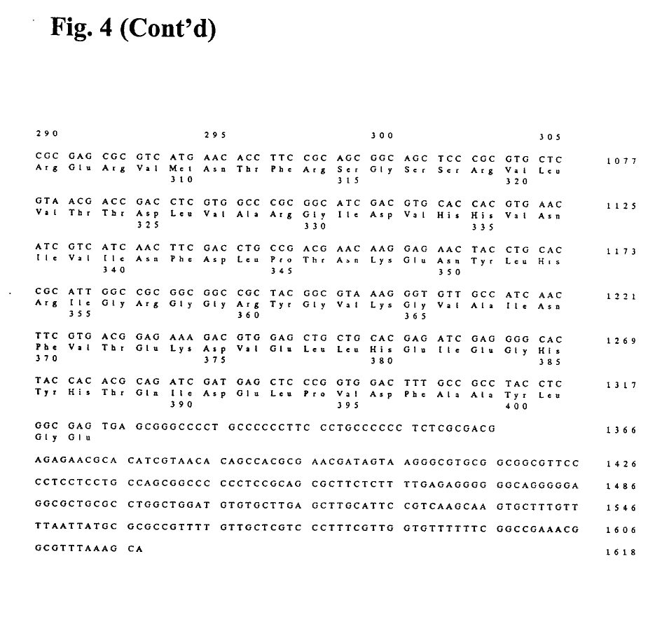

SEQ ID NO:7 and 8: Ra35, the N-terminus of MTB32A (Ra35FL), comprising at least about 195 amino acids from the N-terminus of MTB32A from M. tuberculosis, the nucleotide and amino acid sequence of which is disclosed in FIG. 4 (see also amino acids 33-227 of SEQ ID NO:2 and amino acids 8 to 202 of SEQ ID NO:4). The term Ra35 (N-term) also includes Ra35 amino acid sequences in which any one of the three amino acids at the active site triad (i.e., His, Asp, or Ser) has been changed as described above.

SEQ ID NO:9 and 10: MTBRa12, the C-terminus of MTB32A (Ra35FL), comprising at least about 132 amino acids from the C-terminus of MTB32A from M. tuberculosis (see, e.g., amino acids 224 to 355 of SEQ ID NO:2 and amino acids 199 to 330 of SEQ ID NO:4), the sequence of which is disclosed as SEQ ID NO:4 (DNA) and SEQ ID NO:66 (predicted amino acid sequence) in the U.S. patent application Ser. No. 09/072,967.

SEQ ID NO:11 12, 13. and 14: MTB39 (TbH9), the sequence of which is disclosed as SEQ ID NO:106 (cDNA full length) and SEQ ID NO:107 (protein full length) in the U.S. patent applications Ser. No. 08/658,800, No. 08/659,683, No. 08/818,112, and No. 08/818,111 and in the WO97/09428 and WO97/09429 applications. The sequence is also disclosed as SEQ ID NO:33 (DNA) and SEQ ID NO:91 (amino acid) in U.S. patent application Ser. No. 09/056,559.

The following provides sequences of some fusion Mycobacterium sp. proteins of the invention

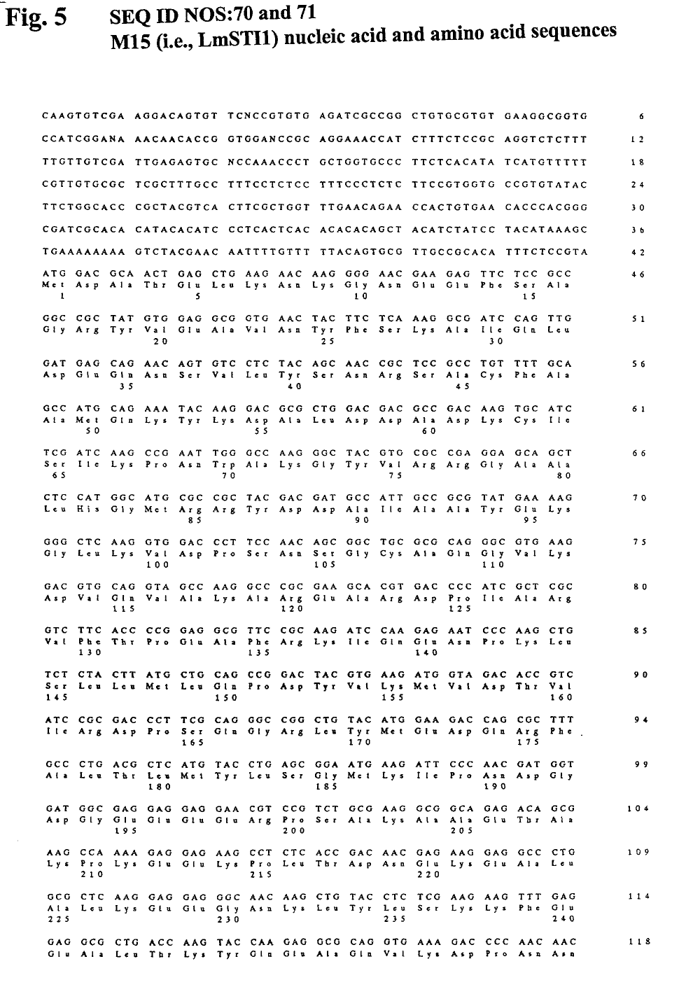

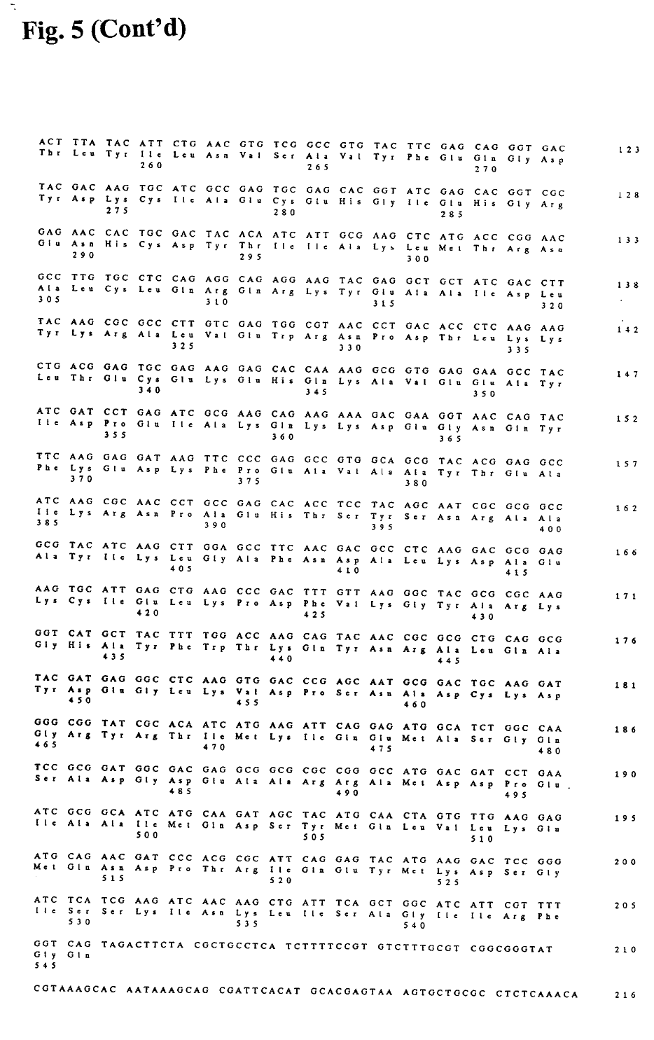

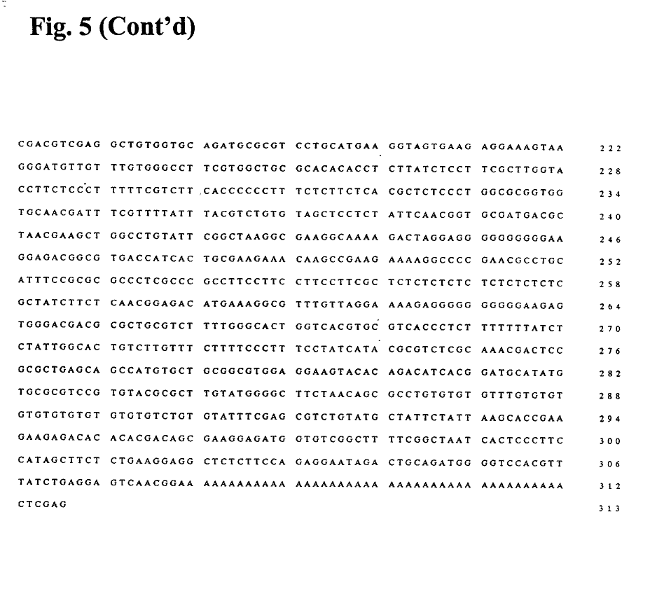

SEQ ID NO:15 and 16: MTB72F (Ra12-TbH9-Ra35), the sequence of which is disclosed as SEQ ID NO: 1 (DNA) and SEQ ID NO:2 (protein) in the U.S. patent application Ser. No. 09/223,040, No. 09/223,040, and in the PCT/US99/07717 application. The term MTB372F also includes MTB72F amino acid sequences in which any one of the three amino acids at the active site triad in Ra35FL (i.e., His, Asp, or Ser), has been changed as described above (see, e.g., MTB72FMutSA, FIG. 5).

SEQ ID NO:17 and 18: MTB72FMutSA (Ra12-TbH9-Ra35MutSA), wherein, in the Ra35 component of the fusion protein, the serine at position 710 has been changed to an alanine.

SEQ ID NO:19 and 20: TbH9-Ra35 (MTB59F), the sequence of which is disclosed as SEQ ID NO:23 (cDNA) and SEQ ID NO:24 (protein) in the U.S. patent application Ser. No. 09/287,849 and in the PCT/US99/07717 application.

The following provides sequences of some additional antigens used in the compositions and fusion proteins of the invention:

SEQ ID NO: 21 and 22: MTB8.4 (DPV), the sequence of which is disclosed as SEQ ID NO:101 (cDNA) and SEQ ID NO:102 (protein) in the U.S. patent applications Ser. No. 08/658,800, No. 08/659,683, No. 08/818,112 and No. 08/818,111 and in the WO97/09428 and WO97/09429 applications.

SEQ ID NO:23 and 24: MTB9.8 (MSL), the sequence of which is disclosed as SEQ ID NO: 12 (DNA), SEQ ID NO:109 (predicted amino acid sequence) and SEQ ID NO:110 to 124 (peptides) in the U.S. patent applications Ser. No. 08/859,381, No. 08/858,998, No. 09/073,009 and No. 09/073,010 and in the PCT/US98/10407 and PCT/US98/10514 applications.

SEQ ID NO:25, 26, and 27: MTB9.9A (MTI, also known as MTI-A), the sequence of which is disclosed as SEQ ID NO:3 and SEQ ID NO:4 (DNA) and SEQ ID NO:29 and SEQ ID NO:51 to 66 (ORF peptide for MTI) in the U.S. patent applications Ser. No. 08/859,381, No. 08/858,998, No. 09/073,009 and vO9/073,010 and in the PCT/US98/10407 and PCT/US98/10514 applications. Two other MTI variants also exist, called MTI-B and MTI-C.

SEQ ID NO:28 and 29: MTB40 (HTCC#l), the sequence of which is disclosed as SEQ ID NO: 137 (cDNA) and 138 (predicted amino acid sequence) in the U.S. patent applications Ser. No. 09/073,009 and No. 09/073,010 and in the PCT/US98/10407 and PCT/US98/10514 applications.

SEQ ID NO:30 and 31: MTB41 (MTCC#2), the sequence of which is disclosed as SEQ ID NO:140 (cDNA) and SEQ ID NO:142 (predicted amino acid sequence) in the U.S. patent applications Ser. No. 09/073,009 and No. 09/073,010 and in the PCT/US98/10407 and PCT/US98/10514 applications.

SEQ ID NO:32 and 33: ESAT-6, the sequence of which is disclosed as SEQ ID NO:103 (DNA) and SEQ ID NO:104 (predicted amino acid sequence) in the U.S. patent application Ser. No. 09/072,967. The sequence of ESAT-6 is also disclosed in U.S. Pat. No. 5,955,077.

SEQ ID NO:34 and 35: Tb38-1 or 38-1 (MTb11), the sequence of which is disclosed in SEQ ID NO:46 (DNA) and SEQ ID NO:88 (predicted amino acid) in the U.S. patent application Ser. Nos. 09/072,96; 08/523,436; 08/523,435; 08/818,112; and 08/818,111; and in the WO97/09428 and WO97/09429 applications.

SEQ ID NO:36 and 37: TbRa3, the sequence of which is disclosed in SEQ ID NO:15 (DNA) and SEQ ID NO:77 (predicted amino acid sequence) of WO 97/09428 and WO97/09429 applications.

SEQ ID NO:38 and 39: 38 kD, the sequence of which is disclosed in SEQ ID NO:154 (DNA) and SEQ ID NO:155 predicted amino acid sequence) in the U.S. patent application Ser. No. 09/072,967. 38 kD has two alternative forms, with and without the N-terminal cysteine residue.

SEQ ID NO:40 and 41: DPEP, the sequence of which is disclosed in SEQ ID NO:52 (DNA) and SEQ ID NO:53 (predicted amino acid sequence) in the WO97/09428 and WO97/09429 publications.

SEQ ID NO:42 and 43: TbH4, the sequence of which is disclosed as SEQ ID NO:43 (DNA) and SEQ ID NO:81 (predicted amino acid sequence) in WO97/09428 and WO97/09429 publications.

SEQ ID NO:44 and 45: DPPD, the sequence of which is disclosed in SEQ ID NO:240 (DNA) and SEQ ID NO:241 (predicted amnino acid sequence) in U.S. Ser. No. 09/072,967 and in the PCT/US99/03268 and PCT/US99/03265 applications. The secreted form of DPPD is shown herein in FIG. 12 of PCT/US00/28095.

MTb82 (MTb867), the sequence of which is disclosed in FIGS. 8 (DNA) and 9 (amino acid) of PCT/US00/2809.

Erd14 (MTb16), the cDNA and amino acids sequences of which are disclosed in Verbon et al., J. Bacteriology 174:1352-1359 (1992).

α-crystalline antigen, the sequence of which is disclosed in Verbon et al., J. Bact. 174:1352-1359 (1992);

85 complex antigen, the sequence of which is disclosed in Content et al., Infect. & Immunol. 59:3205-3212 (1991).

The following provides sequences of some additional fusion proteins used in the compositions and fusion proteins of the invention:

SEQ ID NO:46 and 47: DPV-MTI-MSL-MTCC#2 (MTb71F), the sequence of which is disclosed as SEQ ID NO:15 (nucleic acid) and in SEQ ID NO:16: (protein) in the U.S. patent application Ser. No. 09/287,849 and in the PCT/US99/07717 application.

SEQ ID NO:48 and 49: DPV-MTI-MSL (MTb31F), the sequence of which is disclosed in SEQ ID NO:18 (cDNA) and SEQ ID NO:19 (protein) in the U.S. patent application Ser. No. 09/287,849 and in the PCT/US99/07717 application.

Each of the above sequences is also disclosed in Cole et al. Nature 393:537 (1998) and can be found at, e.g., http://www.sanger.ac.uk and http:/www.pasteur.fr/mycdb/.

The above sequences are disclosed in U.S. patent applications Ser. Nos. 08/523,435, 08/523,436, 08/658,800, 08/659,683, 08/818,111, 08/818,112, 08/942,341, 08/942,578, 08/858,998, 08/859,381, 09/056,556, 09/072,596, 09/072,967, 09/073,009, 09/073,010, 09/223,040, 09/287,849 09/597,796; and in PCT patent applications PCT/US00/28095; PCT/US98/10407, PCT/US98/10514, PCT/US99/03265, PCT/US99/03268, PCT/US99/07717, WO97/09428 and WO97/09429, WO98/16645, WO98/16646, each of which is herein incorporated by reference.

In the nomenclature of the application, Ra35 refers to the N-terminus of MTB32A (Ra35FL), comprising at least about 195 to 205 amino acids of MTB32A from M. tuberculosis, or the corresponding region from another Mycobacterium species. Ra12 refers to the C-terminus of MTB32A (Ra35FL), comprising at least about the last 132 amino acids from MTB32A from M. tuberculosis, or the corresponding region from another Mycobacterium species.

3. Examples of Fusion Between Leishmania and Mycobacterium Sequences

The following provides sequences of fusion nucleic acid constructs between a Leishmania TSA polynucleotide and a Mycobacterium sp. polynucleotide, and proteins encoded by the fusion polynucleotides:

SEQ ID NO:50 and 51: MAPS-DPVpET is a fusion DNA construct comprising Leishmania gene TSA at the N-terminus and linked with the TB antigen DPV (aka MTB8.4). SEQ ID NO:50 is a nucleotide sequence and SEQ ID NO:51 is the corresponding amino acid sequence. This construct is used for protein expression.

SEQ ID NO:52 and 53: MAPS-DPASpET is a fusion DNA construct comprising Leishmania gene TSA at the N-terminus and linked with the TB antigen DPAS (aka MTB12). SEQ ID NO:52 is a nucleotide sequence and SEQ ID NO:53 is the corresponding amino acid sequence. This construct is used for protein expression.

SEQ ID NO:54: MAPS-DPVpc is a fusion DNA vaccine construct comprising Leishmania gene TSA at the N-terminus and linked with the TB antigen DPV (aka MTB8.4).

SEQ ID NO:55: MAPS-DPASpc is a fusion DNA vaccine construct comprising Leishmania gene TSA at the N-terminus and linked with the TB antigen DPAS (aka MTB12).

SEQ ID NO:56 and 57: MAPS-DPV-AC is a fusion construct comprising Leishmania TSA at the N-terminus and linked with the TB antigen DPV (aka MTB8.4) which is codon optimized for expression in eukaryotic cells. SEQ ID NO:56 is a nucleotide sequence, and SEQ ID NO:57 is the corresponding amino acid sequence.

SEQ ID NO:58 and 59: MAPS-DPAS-AC is a fusion construct comprising Leishmania TSA at the N-terminus and linked with the TB antigen DPAS (aka MTB 12) which is codon optimized for expression in eukaryotic cells. SEQ ID NO:58 is a nucleotide sequence, and SEQ ID NO:59 is the corresponding amino acid sequence.

SEQ ID NO:60 and 61: MAPS(N5)-DPV-AC is a fusion construct comprising the first five amino acids of Leishmania TSA at the N-terminus and linked with the TB antigen DPV (aka MTB8.4) which is codon optimized for expression in eukaryotic cells. SEQ ID NO:60 is a nucleotide sequence, and SEQ ID NO:61 is the corresponding amino acid sequence.

SEQ ID NO:62 and 63: MAPS(N10)-DPV-AC is a fusion construct comprising the first ten amino acids of Leishmania TSA at the N-terminus and linked with the TB antigen DPV (aka MTB8.4) which is codon optimized for expression in eukaryotic cells. SEQ ID NO:62 is a nucleotide sequence, and SEQ ID NO:63 is the corresponding amino acid sequence.

SEQ ID NO:64 and 65: MTB72F-MAPS (aka r95f) is a fusion construct comprising a MTB72F (a 72 kDa poly-protein fusion construct comprising Ra12-TbH9-Ra35) linked to the Leishmania TSA. SEQ ID NO:64 is a nucleotide sequence, and SEQ ID NO:65 is the corresponding amino acid sequence.

These fusion constructs are merely exemplary, and one of skill in the art would readily recognize that any suitable Leishmania sequences can be linked to any other heterologous sequences of interest, particularly those derived from pathogenic agents.

### Polynucleotide Identification and Characterization

The above-described polynucleotides may be identified, prepared and/or manipulated using any of a variety of well established techniques. For example, a polynucleotide may be identified, as described in more detail below, by screening a microarray of cDNAs for tumor-associated expression (i.e., expression that is at least two fold greater in a tumor than in normal tissue, as determined using a representative assay provided herein). Such screens may be performed, for example, using a Synteni microarray (Palo Alto, Calif.) according to the manufacturer's instructions (and essentially as described by Schena et al., Proc. Natl. Acad. Sci. USA 93:10614-10619 (1996) and Heller et al., Proc. Natl. Acad. Sci. USA 94:2150-2155 (1997)). Alternatively, polynucleotides may be amplified from cDNA prepared from cells expressing the proteins described herein, such as M. tuberculosis or Leishmania cells. Such polynucleotides may be amplified via polymerase chain reaction (PCR). For this approach, sequence-specific primers may be designed based on the sequences provided herein, and may be purchased or synthesized.

An amplified portion of a polynucleotide of the present invention may be used to isolate a full length gene from a suitable library (e.g., a M. tuberculosis cDNA library) using well known techniques. Within such techniques, a library (cDNA or genomic) is screened using one or more polynucleotide probes or primers suitable for amplification. Preferably, a library is size-selected to include larger molecules. Random primed libraries may also be preferred for identifying 5′ and upstream regions of genes. Genomic libraries are preferred for obtaining introns and extending 5′ sequences.

For hybridization techniques, a partial sequence may be labeled (e.g., by nick-translation or end-labeling with ³²P) using well known techniques. A bacterial or bacteriophage library is then generally screened by hybridizing filters containing denatured bacterial colonies (or lawns containing phage plaques) with the labeled probe (see Sambrook et al., Molecular Cloning: A Laboratory Manual (1989)). Hybridizing colonies or plaques are selected and expanded, and the DNA is isolated for further analysis. cDNA clones may be analyzed to determine the amount of additional sequence by, for example, PCR using a primer from the partial sequence and a primer from the vector. Restriction maps and partial sequences may be generated to identify one or more overlapping clones. The complete sequence may then be determined using standard techniques, which may involve generating a series of deletion clones. The resulting overlapping sequences can then assembled into a single contiguous sequence. A full length cDNA molecule can be generated by ligating suitable fragments, using well known techniques.

Alternatively, there are numerous amplification techniques for obtaining a full length coding sequence from a partial cDNA sequence. Within such techniques, amplification is generally performed via PCR. Any of a variety of commercially available kits may be used to perform the amplification step. Primers may be designed using, for example, software well known in the art. Primers are preferably 22-30 nucleotides in length, have a GC content of at least 50% and anneal to the target sequence at temperatures of about 68° C. to 72° C. The amplified region may be sequenced as described above, and overlapping sequences assembled into a contiguous sequence.

One such amplification technique is inverse PCR (see Triglia et al., Nucl. Acids Res. 16:8186 (1988)), which uses restriction enzymes to generate a fragment in the known region of the gene. The fragment is then circularized by intramolecular ligation and used as a template for PCR with divergent primers derived from the known region. Within an alternative approach, sequences adjacent to a partial sequence may be retrieved by amplification with a primer to a linker sequence and a primer specific to a known region. The amplified sequences are typically subjected to a second round of amplification with the same linker primer and a second primer specific to the known region. A variation on this procedure, which employs two primers that initiate extension in opposite directions from the known sequence, is described in WO 96/38591. Another such technique is known as “rapid amplification of cDNA ends” or RACE. This technique involves the use of an internal primer and an external primer, which hybridizes to a polyA region or vector sequence, to identify sequences that are 5′ and 3′ of a known sequence. Additional techniques include capture PCR (Lagerstrom et al., PCR Methods Applic. 1:111- 19 (1991)) and walking PCR (Parker et al., Nucl. Acids. Res. 19:3055-60 (1991)). Other methods employing amplification may also be employed to obtain a full length cDNA sequence.

In certain instances, it is possible to obtain a full length cDNA sequence by analysis of sequences provided in an expressed sequence tag (EST) database, such as that available from GenBank. Searches for overlapping ESTs may generally be performed using well known programs (e.g., NCBI BLAST searches), and such ESTs may be used to generate a contiguous full length sequence. Full length DNA sequences may also be obtained by analysis of genomic fragments.

### Polynucleotide Expression in Host Cells

In other embodiments of the invention, Leishmania fusion constructs may be used in recombinant DNA molecules to direct expression of a polypeptide in appropriate host cells. Due to the inherent degeneracy of the genetic code, other DNA sequences that encode substantially the same or a functionally equivalent amino acid sequence may be produced and these sequences may be used to clone and express a given polypeptide.

As will be understood by those of skill in the art, it may be advantageous in some instances to produce polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence.

Moreover, the polynucleotide sequences of the present invention can be engineered using methods generally known in the art in order to alter polypeptide encoding sequences for a variety of reasons, including but not limited to, alterations which modify the cloning, processing, and/or expression of the gene product. For example, DNA shuffling by random fragmentation and PCR reassembly of gene fragments and synthetic oligonucleotides may be used to engineer the nucleotide sequences. In addition, site-directed mutagenesis may be used to insert new restriction sites, alter glycosylation patterns, change codon preference, produce splice variants, or introduce mutations, and so forth.

In another embodiment of the invention, natural, modified, or recombinant nucleic acid sequences may be ligated to a heterologous sequence to encode a fusion protein. For example, to screen peptide libraries for inhibitors of polypeptide activity, it may be useful to encode a chimeric protein that can be recognized by a commercially available antibody. A fusion protein may also be engineered to contain a cleavage site located between the polypeptide-encoding sequence and the heterologous protein sequence, so that the polypeptide may be cleaved and purified away from the heterologous moiety.

Sequences encoding a desired polypeptide may be synthesized, in whole or in part, using chemical methods well known in the art (see Caruthers, M. H. et al., Nucl. Acids Res. Symp. Ser. pp. 215-223 (1980), Horn et al., Nucl. Acids Res. Symp. Ser. pp. 225-232 (1980)). Alternatively, the protein itself may be produced using chemical methods to synthesize the amino acid sequence of a polypeptide, or a portion thereof. For example, peptide synthesis can be performed using various solid-phase techniques (Roberge et al., Science 269:202-204 (1995)) and automated synthesis may be achieved, for example, using the ABI 431A Peptide Synthesizer (Perkin Elmer, Palo Alto, Calif.).

A newly synthesized peptide may be substantially purified by preparative high performance liquid chromatography (e.g., Creighton, Proteins, Structures and Molecular Principles (1983)) or other comparable techniques available in the art. The composition of the synthetic peptides may be confirmed by amino acid analysis or sequencing (e.g., the Edman degradation procedure). Additionally, the amino acid sequence of a polypeptide, or any part thereof, may be altered during direct synthesis and/or combined using chemical methods with sequences from other proteins, or any part thereof, to produce a variant polypeptide.

In order to express a desired polypeptide, the nucleotide sequences encoding the polypeptide, or functional equivalents, may be inserted into appropriate expression vector, i.e., a vector which contains the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook et al., Molecular Cloning, A Laboratory Manual (1989), and Ausubel et al., Current Protocols in Molecular Biology (1989).

A variety of expression vector/host systems may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

The “control elements” or “regulatory sequences” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the PBLUESCRIPT phagemid (Stratagene, La Jolla, Calif.) or PSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, a number of expression vectors may be selected depending upon the use intended for the expressed polypeptide. For example, when large quantities are needed, for example for the induction of antibodies, vectors which direct high level expression of fusion proteins that are readily purified may be used. Such vectors include, but are not limited to, the multifunctional E. coli cloning and expression vectors such as BLUESCRIPT (Stratagene), in which the sequence encoding the polypeptide of interest may be ligated into the vector in frame with sequences for the amino-terminal Met and the subsequent 7 residues of β-galactosidase so that a hybrid protein is produced; pIN vectors (Van Heeke & Schuster, J. Biol. Chem. 264:5503-5509 (1989)); and the like. pGEX Vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase, and PGH may be used. For reviews, see Ausubel et al. (supra) and Grant et al., Methods Enzymol. 153:516-544 (1987).

In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu, EMBO J. 6:307-311 (1987)). Alternatively, plant promoters such as the small subunit of RUBISCO or heat shock promoters may be used (Coruzzi et al., EMBO J. 3:1671-1680 (1984); Broglie et al., Science 224:838-843 (1984); and Winter et al., Results Probl. Cell Differ. 17:85-105 (1991)). These constructs can be introduced into plant cells by direct DNA transformation or pathogen-mediated transfection. Such techniques are described in a number of generally available reviews (see, e.g., Hobbs in McGraw Hill Yearbook of Science and Technology pp. 191-196 (1992)).

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia larvae. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. frugiperda cells or Trichoplusia larvae in which the polypeptide of interest may be expressed (Engelhard et al., Proc. Natl. Acad. Sci. U.S.A. 91:3224-3227 (1994)).

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan & Shenk, Proc. Natl. Acad. Sci. U.S.A. 81:3655-3659 (1984)). In addition, transcription enhancers, such as the Rous sarcoma virus (RSV) enhancer, may be used to increase expression in mammalian host cells.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf. et al., Results Probl. Cell Differ. 20:125-162 (1994)).

In addition, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells such as CHO, HeLa, MDCK, HEK293, and W138, which have specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines which stably express a polynucleotide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase (Wigler et al., Cell 11:223-32 (1977)) and adenine phosphoribosyltransferase (Lowy et al., Cell 22:817-23 (1990)) genes which can be employed in tk⁻ or aprt⁻ cells, respectively. Also, antimetabolite, antibiotic or herbicide resistance can be used as the basis for selection; for example, dhfr which confers resistance to methotrexate (Wigler et al., Proc. Natl. Acad. Sci. U.S.A. 77:3567-70 (1980)); npt, which confers resistance to the aminoglycosides, neomycin and G-418 (Colbere-Garapin et al., J. Mol. Biol. 150:1-14 (1981)); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman & Mulligan, Proc. Natl. Acad. Sci. U.S.A. 85:8047-51 (1988)). Recently, the use of visible markers has gained popularity with such markers as anthocyanins, β-glucuronidase and its substrate GUS, and luciferase and its substrate luciferin, being widely used not only to identify transformants, but also to quantify the amount of transient or stable protein expression attributable to a specific vector system (Rhodes et al., Methods Mol. Biol. 55:121-131 (1995)).

Although the presence/absence of marker gene expression suggests that the gene of interest is also present, its presence and expression may need to be confirmed. For example, if the sequence encoding a polypeptide is inserted within a marker gene sequence, recombinant cells containing sequences can be identified by the absence of marker gene function. Alternatively, a marker gene can be placed in tandem with a polypeptide-encoding sequence under the control of a single promoter. Expression of the marker gene in response to induction or selection usually indicates expression of the tandem gene as well.

Alternatively, host cells which contain and express a desired polynucleotide sequence may be identified by a variety of procedures known to those of skill in the art. These procedures include, but are not limited to, DNA-DNA or DNA-RNA hybridizations and protein bioassay or immunoassay techniques which include membrane, solution, or chip based technologies for the detection and/or quantification of nucleic acid or protein.

A variety of protocols for detecting and measuring the expression of polynucleotide-encoded products, using either polyclonal or monoclonal antibodies specific for the product are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on a given polypeptide may be preferred for some applications, but a competitive binding assay may also be employed. These and other assays are described, among other places, in Hampton et al., Serological Methods, a Laboratory Manual (1990) and Maddox et al., J. Exp. Med. 158:1211-1216 (1983).

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with a polynucleotide sequence of interest may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides of the invention may be designed to contain signal sequences which direct secretion of the encoded polypeptide through a prokaryotic or eukaryotic cell membrane. Other recombinant constructions may be used to join sequences encoding a polypeptide of interest to nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences such as those specific for Factor XA or enterokinase (Invitrogen. San Diego, Calif.) between the purification domain and the encoded polypeptide may be used to facilitate purification. One such expression vector provides for expression of a fusion protein containing a polypeptide of interest and a nucleic acid encoding 6 histidine residues preceding a thioredoxin or an enterokinase cleavage site. The histidine residues facilitate purification on IMIAC (immobilized metal ion affinity chromatography) as described in Porath et al., Prot. Exp. Purif. 3:263-281 (1992) while the enterokinase cleavage site provides a means for purifying the desired polypeptide from the fusion protein. A discussion of vectors which contain fusion proteins is provided in Kroll et al., DNA Cell Biol. 12:441-453 (1993)).

In addition to recombinant production methods, polypeptides of the invention, and fragments thereof, may be produced by direct peptide synthesis using solid-phase techniques (Merrifield, J. Am. Chem. Soc. 85:2149-2154 (1963)). Protein synthesis may be performed using manual techniques or by automation. Automated synthesis may be achieved, for example, using Applied Biosystems 431A Peptide Synthesizer (Perkin Elmer). Alternatively, various fragments may be chemically synthesized separately and combined using chemical methods to produce the full length molecule.

### In Vivo Polynucleotide Delivery Techniques

In additional embodiments, genetic constructs comprising one or more of the polynucleotides of the invention are introduced into cells in vivo. This may be achieved using any of a variety or well known approaches, several of which are outlined below for the purpose of illustration.

I. Adenovirus

One of the preferred methods for in vivo delivery of one or more nucleic acid sequences involves the use of an adenovirus expression vector. “Adenovirus expression vector” is meant to include those constructs containing adenovirus sequences sufficient to (a) support packaging of the construct and (b) to express a polynucleotide that has been cloned therein in a sense or antisense orientation. Of course, in the context of an antisense construct, expression does not require that the gene product be synthesized.

The expression vector comprises a genetically engineered form of an adenovirus. Knowledge of the genetic organization of adenovirus, a 36 kb, linear, double-stranded DNA virus, allows substitution of large pieces of adenoviral DNA with foreign sequences up to 7 kb (Grunhaus & Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones & Shenk, 1978), the current adenovirus vectors, with the help of 293 cells, carry foreign DNA in either the E1, the D3 or both regions (Graham & Prevec, 1991). In nature, adenovirus can package approximately 105% of the wild-type genome (Ghosh-Choudhury et al., 1987), providing capacity for about 2 extra kB of DNA. Combined with the approximately 5.5 kB of DNA that is replaceable in the E1 and E3 regions, the maximum capacity of the current adenovirus vector is under 7.5 kB, or about 15% of the total length of the vector. More than 80% of the adenovirus viral genome remains in the vector backbone and is the source of vector-borne cytotoxicity. Also, the replication deficiency of the E1-deleted virus is incomplete. For example, leakage of viral gene expression has been observed with the currently available vectors at high multiplicities of infection (MOI) (Mulligan, 1993).

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the currently preferred helper cell line is 293.

Recently, Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain a conditional replication-defective adenovirus vector for use in the present invention, since Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus & Horwitz, 1992; Graham & Prevec, 1992). Recently, animal studies suggested that recombinant adenovirus could be used for gene therapy (Stratford-Perricaudet & Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz & Gerard, 1993) and stereotactic inoculation into the brain (Le Gal La Salle et al., 1993).

2. Retroviruses

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding one or more oligonucleotide or polynucleotide sequences of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas & Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

3. Adeno-Associated Viruses

AAV (Ridgeway, 1988; Hermonat & Muzycska, 1984) is a parovirus, discovered as a contamination of adenoviral stocks. It is a ubiquitous virus (antibodies are present in 85% of the US human population) that has not been linked to any disease. It is also classified as a dependovirus, because its replications is dependent on the presence of a helper virus, such as adenovirus. Five serotypes have been isolated, of which AAV-2 is the best characterized. AAV has a single-stranded linear DNA that is encapsidated into capsid proteins VP1, VP2 and VP3 to form an icosahedral virion of 20 to 24 nm in diameter (Muzyczka & McLaughlin, 1988).

The AAV DNA is approximately 4.7 kilobases long. It contains two open reading frames and is flanked by two ITRs. There are two major genes in the AAV genome: rep and cap. The rep gene codes for proteins responsible for viral replications, whereas cap codes for capsid protein VP1-3. Each ITR forms a T-shaped hairpin structure. These terminal repeats are the only essential cis components of the AAV for chromosomal integration. Therefore, the AAV can be used as a vector with all viral coding sequences removed and replaced by the cassette of genes for delivery. Three viral promoters have been identified and named p5, p19, and p40, according to their map position. Transcription from p5 and p19 results in production of rep proteins, and transcription from p40 produces the capsid proteins (Hermonat & Muzyczka, 1984).

There are several factors that prompted researchers to study the possibility of using rAAV as an expression vector One is that the requirements for delivering a gene to integrate into the host chromosome are surprisingly few. It is necessary to have the 145-bp ITRs, which are only 6% of the AAV genome. This leaves room in the vector to assemble a 4.5-kb DNA insertion. While this carrying capacity may prevent the AAV from delivering large genes, it is amply suited for delivering the antisense constructs of the present invention.

AAV is also a good choice of delivery vehicles due to its safety. There is a relatively complicated rescue mechanism: not only wild type adenovirus but also AAV genes are required to mobilize rAAV. Likewise, AAV is not pathogenic and not associated with any disease. The removal of viral coding sequences minimizes immune reactions to viral gene expression, and therefore, rAAV does not evoke an inflammatory response.

4. Other Viral Vectors as Expressed Constructs

Other viral vectors may be employed as expression constructs in the present invention for the delivery of oligonucleotide or polynucleotide sequences to a host cell. Vectors derived from viruses such as vaccinia virus (Ridgeway, 1988; Coupar et al., 1988), lentiviruses, polio viruses and herpes viruses may be employed. They offer several attractive features for various mammalian cells (Friedmann, 1989; Ridgeway, 1988; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. The hepatotropism and persistence (integration) were particularly attractive properties for liver-directed gene transfer. Chang et al. (1991) introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

5. Non-Viral Vectors

In order to effect expression of the oligonucleotide or polynucleotide sequences of the present invention, the expression construct must be delivered into a cell. This delivery may be accomplished in vitro, as in laboratory procedures for transforming cells lines, or in vivo or ex vivo, as in the treatment of certain disease states. As described above, one preferred mechanism for delivery is via viral infection where the expression construct is encapsulated in an infectious viral particle.

Once the expression construct has been delivered into the cell the nucleic acid encoding the desired oligonucleotide or polynucleotide sequences may be positioned and expressed at different sites. In certain embodiments, the nucleic acid encoding the construct may be stably integrated into the genome of the cell. This integration may be in the specific location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In certain embodiments of the invention, the expression construct comprising one or more oligonucleotide or polynucleotide sequences may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty & Reshef (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

Another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

### Polypeptides

The present invention, in other aspects, provides fusion polypeptides and compositions comprising the fusion polypeptides. Generally, a polypeptide of the invention will be an isolated polypeptide (or an epitope, variant, or active fragment thereof) derived from a mammalian species. Preferably, the polypeptide is encoded by a polynucleotide sequence disclosed herein or a sequence which hybridizes under moderately stringent conditions to a polynucleotide sequence disclosed herein. Alternatively, the polypeptide may be defined as a polypeptide which comprises a contiguous amino acid sequence from an amino acid sequence disclosed herein, or which polypeptide comprises an entire amino acid sequence disclosed herein.

Immunogenic portions may generally be identified using well known techniques, such as those summarized in Paul, Fundamental Immunology, 3rd ed., 243-247 (1993) and references cited therein. Such techniques include screening polypeptides for the ability to react with antigen-specific antibodies, antisera and/or T-cell lines or clones. As used herein, antisera and antibodies are “antigen-specific” if they specifically bind to an antigen (i.e., they react with the protein in an ELISA or other immunoassay, and do not react detectably with unrelated proteins). Such antisera and antibodies may be prepared as described herein, and using well known techniques. An immunogenic portion of a Mycobacterium sp. protein is a portion that reacts with such antisera and/or T-cells at a level that is not substantially less than the reactivity of the full length polypeptide (e.g., in an ELISA and/or T-cell reactivity assay). Similarly, an immunogenic portion of a Leishmania protein is a portion that reacts with such antisera and/or T-cells at a level that is not substantially less than the reactivity of the full length polypeptide. Such immunogenic portions may react within such assays at a level that is similar to or greater than the reactivity of the full length polypeptide. Such screens may generally be performed using methods well known to those of ordinary skill in the art, such as those described in Harlow & Lane, Antibodies: A Laboratory Manual (1988). For example, a polypeptide may be immobilized on a solid support and contacted with patient sera to allow binding of antibodies within the sera to the immobilized polypeptide. Unbound sera may then be removed and bound antibodies detected using, for example, ¹²⁵I-labeled Protein A.

Polypeptides may be prepared using any of a variety of well known techniques. Recombinant polypeptides encoded by DNA sequences as described above may be readily prepared from the DNA sequences using any of a variety of expression vectors known to those of ordinary skill in the art. Expression may be achieved in any appropriate host cell that has been transformed or transfected with an expression vector containing a DNA molecule that encodes a recombinant polypeptide. Suitable host cells include prokaryotes, yeast, and higher eukaryotic cells, such as mammalian cells and plant cells. Preferably, the host cells employed are E. coli, yeast or a mammalian cell line such as COS or CHO. Supernatants from suitable host/vector systems which secrete recombinant protein or polypeptide into culture media may be first concentrated using a commercially available filter. Following concentration, the concentrate may be applied to a suitable purification matrix such as an affinity matrix or an ion exchange resin. Finally, one or more reverse phase HPLC steps can be employed to further purify a recombinant polypeptide.

Polypeptides of the invention, immunogenic fragments thereof, and other variants having less than about 100 amino acids, and generally less than about 50 amino acids, may also be generated by synthetic means, using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146 (1963). Equipment for automated synthesis of polypeptides is commercially available from suppliers such as Perkin E1mer/Applied BioSystems Division (Foster City, Calif.), and may be operated according to the manufacturer's instructions.

In embodiments of the invention, a fusion polypeptide comprises Leishmania TSA, LeIF, M15 or 6H polypeptide or a fragment thereof and a heterologous polypeptide. Any heterologous polypeptide of interest can be fused to the Leishmania polypeptide. Typically, the heterologous polypeptide is a HIV antigen, a malaria antigen, a cancer antigen, a viral antigen or a bacterial antigen. Preferably, the heterologous polypeptide is a Mycobacterium antigen or an antigenic fragment thereof. For example, the fusion partner to the Leishmania polypeptide is selected from Mycobacterium antigens or antigenic fragments thereof, such as MTB8.4 antigen, MTB9.8 antigen, MTB9.9 antigen, MTB12 antigen, MTB32A antigen, MTB40 antigen, MTB41 antigen, TbH9 antigen, Ra35 antigen, Ra12 antigen, 38-1 antigen, TbRa3 antigen, 38 kD antigen, DPEP antigen, TbH4 antigen, DPPD antigen, MTB82 antigen, Erd14 antigen, ESAT-6 antigen, MTB85 complex antigen, MTB59F antigen, MTB72F antigen, MTB31F antigen, or MTB71F antigen.

Fusion polypeptides of the present invention can further comprise one or more additional polypeptides. For example, an additional fusion partner may assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the protein or to enable the protein to be targeted to desired intracellular compartments. Still further fusion partners include affinity tags, which facilitate purification of the protein.

Within preferred embodiments, an additional immunological fusion partner is derived from protein D, a surface protein of the gram-negative bacterium Haemophilus influenza B (WO 91/18926). Preferably, a protein D derivative comprises approximately the first third of the protein (e.g., the first N-terminal 100-110 amino acids), and a protein D derivative may be lipidated. Within certain preferred embodiments, the first 109 residues of a lipoprotein D fusion partner is included on the N-terminus to provide the polypeptide with additional exogenous T-cell epitopes and to increase the expression level in E. coli (thus functioning as an expression enhancer). The lipid tail ensures optimal presentation of the antigen to antigen presenting cells. Other fusion partners include the non-structural protein from influenzae virus, NS1 (hemaglutinin). Typically, the N-terminal 81 amino acids are used, although different fragments that include T-helper epitopes may be used.

In another embodiment, the additional immunological fusion partner is the protein known as LYTA, or a portion thereof (preferably a C-terminal portion). LYTA is derived from Streptococcus pneumoniae, which synthesizes an N-acetyl-L-alanine amidase known as amidase LYTA (encoded by the LytA gene; Gene 43:265-292 (1986)). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus has been described (see Biotechnology 10:795-798 (1992)). Within a preferred embodiment, a repeat portion of LYTA may be incorporated into a fusion protein. A repeat portion is found in the C-terminal region starting at residue 178. A particularly preferred repeat portion incorporates residues 188-305.

Fusion polypeptides of the present invention may generally be prepared using standard techniques, including chemical conjugation. Preferably, a fusion protein is expressed as a recombinant protein, allowing the production of increased levels, relative to a non-fused protein, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides.

A peptide linker sequence may be employed to separate the first and second polypeptide components by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46 (1985); Murphy et al., Proc. Natl. Acad. Sci. USA 83:8258-8262 (1986); U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

In general, polypeptides (including fusion proteins) and polynucleotides as described herein are isolated. An “isolated” polypeptide or polynucleotide is one that is removed from its original environment. For example, a naturally-occurring protein is isolated if it is separated from some or all of the coexisting materials in the natural system. Preferably, such polypeptides are at least about 90% pure, more preferably at least about 95% pure and most preferably at least about 99% pure. A polynucleotide is considered to be isolated if, for example, it is cloned into a vector that is not a part of the natural environment.

### T Cells

Immunotherapeutic compositions may also, or alternatively, comprise T cells specific for a Mycobacterium antigen. Such cells may generally be prepared in vitro or ex vivo, using standard procedures. For example, T cells may be isolated from bone marrow, peripheral blood, or a fraction of bone marrow or peripheral blood of a patient, using a commercially available cell separation system, such as the Isolex™ System, available from Nexell Therapeutics, Inc. (Irvine, Calif.; see also U.S. Pat. No. 5,240,856; U.S. Pat. No. 5,215,926; WO 89/06280; WO 91/16116 and WO 92/07243). Alternatively, T cells may be derived from related or unrelated humans, non-human mammals, cell lines or cultures.

T cells may be stimulated with a polypeptide of the invention, a polynucleotide encoding such a polypeptide, and/or an antigen presenting cell (APC) that expresses such a polypeptide. Such stimulation is performed under conditions and for a time sufficient to permit the generation of T cells that are specific for the polypeptide. Preferably, the polypeptide or polynucleotide is present within a delivery vehicle, such as a microsphere, to facilitate the generation of specific T cells.

T cells are considered to be specific for a polypeptide of the invention if the T cells specifically proliferate, secrete cytokines or kill target cells coated with the polypeptide or expressing a gene encoding the polypeptide. T cell specificity may be evaluated using any of a variety of standard techniques. For example, within a chromium release assay or proliferation assay, a stimulation index of more than two fold increase in lysis and/or proliferation, compared to negative controls, indicates T cell specificity. Such assays may be performed, for example, as described in Chen et al., Cancer Res. 54:1065-1070 (1994). Alternatively, detection of the proliferation of T cells may be accomplished by a variety of known techniques. For example, T cell proliferation can be detected by measuring an increased rate of DNA synthesis (e.g., by pulse-labeling cultures of T cells with tritiated thymidine and measuring the amount of tritiated thymidine incorporated into DNA). Contact with a polypeptide of the invention (100 ng/ml-100 μg/ml, preferably 200 ng/ml-25 μg/ml) for 3-7 days should result in at least a two fold increase in proliferation of the T cells. Contact as described above for 2-3 hours should result in activation of the T cells, as measured using standard cytokine assays in which a two fold increase in the level of cytokine release (e.g., TNF or IFN-γ) is indicative of T cell activation (see Coligan et al., Current Protocols in Immunology, vol. 1 (1998)). T cells that have been activated in response to a polypeptide, polynucleotide or polypeptide-expressing APC may be CD4⁺ and/or CD8⁺. Protein-specific T cells may be expanded using standard techniques. Within preferred embodiments, the T cells are derived from a patient, a related donor or an unrelated donor, and are administered to the patient following stimulation and expansion.

For therapeutic purposes, CD4⁺ or CD8⁺ T cells that proliferate in response to a polypeptide, polynucleotide or APC can be expanded in number either in vitro or in vivo. Proliferation of such T cells in vitro may be accomplished in a variety of ways. For example, the T cells can be re-exposed to a polypeptide, or a short peptide corresponding to an immunogenic portion of such a polypeptide, with or without the addition of T cell growth factors, such as interleukin-2, and/or stimulator cells that synthesize the polypeptide. Alternatively, one or more T cells that proliferate in the presence of the protein can be expanded in number by cloning. Methods for cloning cells are well known in the art, and include limiting dilution.

### Pharmaceutical Compositions

In additional embodiments, the present invention concerns formulation of one or more of the polynucleotide, polypeptide, T-cell and/or antibody compositions disclosed herein in pharmaceutically-acceptable solutions for administration to a cell or an animal, either alone, or in combination with one or more other modalities of therapy.

It will also be understood that, if desired, the nucleic acid segment, RNA, DNA or PNA compositions that express a polypeptide as disclosed herein may be administered in combination with other agents as well, such as, e.g., other proteins or polypeptides or various pharmaceutically-active agents. In fact, there is virtually no limit to other components that may also be included, given that the additional agents do not cause a significant adverse effect upon contact with the target cells or host tissues. The compositions may thus be delivered along with various other agents as required in the particular instance. Such compositions may be purified from host cells or other biological sources, or alternatively may be chemically synthesized as described herein. Likewise, such compositions may further comprise substituted or derivatized RNA or DNA compositions.

Formulation of pharmaceutically-acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, and intramuscular administration and formulation.

1. Oral Delivery

In certain applications, the pharmaceutical compositions disclosed herein may be delivered via oral administration to an animal. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

The active compounds may even be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. No. 5,641,515; U.S. Pat. No. 5,580,579 and U.S. Pat. No. 5,792,451, each specifically incorporated herein by reference in its entirety). The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

Typically, these formulations may contain at least about 0.1% of the active compound or more, although the percentage of the active ingredient(s) may, of course, be varied and may conveniently be between about 1 or 2% and about 60% or 70% or more of the weight or volume of the total formulation. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

2. Injectable Delivery

In certain circumstances it will be desirable to deliver the pharmaceutical compositions disclosed herein parenterally, intravenously, intramuscularly, or even intraperitoneally as described in U.S. Pat. No. 5,543,158; U.S. Pat. No. 5,641,515 and U.S. Pat. No. 5,399,363 (each specifically incorporated herein by reference in its entirety). Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., Remington's Pharmaceutical Sciences, 15th Edition, pp. 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

3. Nasal Delivery

In certain embodiments, the pharmaceutical compositions may be delivered by intranasal sprays, inhalation, and/or other aerosol delivery vehicles. Methods for delivering genes, nucleic acids, and peptide compositions directly to the lungs via nasal aerosol sprays has been described e.g., in U.S. Pat. No. 5,756,353 and U.S. Pat. No. 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

4. Liposome-, Nanocapsule-, and Microparticle-Mediated Delivery

In certain embodiments, the inventors contemplate the use of liposomes, nanocapsules, microparticles, microspheres, lipid particles, vesicles, and the like, for the introduction of the compositions of the present invention into suitable host cells. In particular, the compositions of the present invention may be formulated for delivery either encapsulated in a lipid particle, a liposome, a vesicle, a nanosphere, or a nanoparticle or the like.

Such formulations may be preferred for the introduction of pharmaceutically-acceptable formulations of the nucleic acids or constructs disclosed herein. The formation and use of liposomes is generally known to those of skill in the art (see for example, Couvreur et al., 1977; Couvreur, 1988; Lasic, 1998; which describes the use of liposomes and nanocapsules in the targeted antibiotic therapy for intracellular bacterial infections and diseases). Recently, liposomes were developed with improved serum stability and circulation half-times (Gabizon & Papahadjopoulos, 1988; Allen and Choun, 1987; U.S. Pat. No. 5,741,516, specifically incorporated herein by reference in its entirety). Further, various methods of liposome and liposome like preparations as potential drug carriers have been reviewed (Takakura, 1998; Chandran et al., 1997; Margalit, 1995; U.S. Pat. No. 5,567,434; U.S. Pat. No. 5,552,157; U.S. Pat. No. 5,565,213; U.S. Pat. No. 5,738,868 and U.S. Pat. No. 5,795,587, each specifically incorporated herein by reference in its entirety).

Liposomes have been used successfully with a number of cell types that are normally resistant to transfection by other procedures including T cell suspensions, primary hepatocyte cultures and PC 12 cells (Renneisen et al., 1990; Muller et al., 1990). In addition, liposomes are free of the DNA length constraints that are typical of viral-based delivery systems. Liposomes have been used effectively to introduce genes, drugs (Heath & Martin, 1986; Heath et al., 1986; Balazsovits et al., 1989; Fresta & Puglisi, 1996), radiotherapeutic agents (Pikul et al., 1987), enzymes (Imaizumi et al., 1990a; Imaizumi et al., 1990b), viruses (Faller & Baltimore, 1984), transcription factors and allosteric effectors (Nicolau & Gersonde, 1979) into a variety of cultured cell lines and animals. In addition, several successful clinical trails examining the effectiveness of liposome-mediated drug delivery have been completed (Lopez-Berestein et al., 1985a; 1985b; Coune, 1988; Sculier et al., 1988). Furthermore, several studies suggest that the use of liposomes is not associated with autoimmune responses, toxicity or gonadal localization after systemic delivery (Mori & Fukatsu, 1992).

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Liposomes bear resemblance to cellular membranes and are contemplated for use in connection with the present invention as carriers for the peptide compositions. They are widely suitable as both water- and lipid-soluble substances can be entrapped, i.e. in the aqueous spaces and within the bilayer itself, respectively. It is possible that the drug-bearing liposomes may even be employed for site-specific delivery of active agents by selectively modifying the liposomal formulation.

In addition to the teachings of Couvreur et al. (1977; 1988), the following information may be utilized in generating liposomal formulations. Phospholipids can form a variety of structures other than liposomes when dispersed in water, depending on the molar ratio of lipid to water. At low ratios the liposome is the preferred structure. The physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

In addition to temperature, exposure to proteins can alter the permeability of liposomes. Certain soluble proteins, such as cytochrome c, bind, deform and penetrate the bilayer, thereby causing changes in permeability. Cholesterol inhibits this penetration of proteins, apparently by packing the phospholipids more tightly. It is contemplated that the most useful liposome formations for antibiotic and inhibitor delivery will contain cholesterol.

The ability to trap solutes varies between different types of liposomes. For example, MLVs are moderately efficient at trapping solutes, but SUVs are extremely inefficient. SUVs offer the advantage of homogeneity and reproducibility in size distribution, however, and a compromise between size and trapping efficiency is offered by large unilamellar vesicles (LUVs). These are prepared by ether evaporation and are three to four times more efficient at solute entrapment than MLVs.

In addition to liposome characteristics, an important determinant in entrapping compounds is the physicochemical properties of the compound itself. Polar compounds are trapped in the aqueous spaces and nonpolar compounds bind to the lipid bilayer of the vesicle. Polar compounds are released through permeation or when the bilayer is broken, but nonpolar compounds remain affiliated with the bilayer unless it is disrupted by temperature or exposure to lipoproteins. Both types show maximum efflux rates at the phase transition temperature.

Liposomes interact with cells via four different mechanisms: endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. It often is difficult to determine which mechanism is operative and more than one may operate at the same time.

The fate and disposition of intravenously injected liposomes depend on their physical properties, such as size, fluidity, and surface charge. They may persist in tissues for h or days, depending on their composition, and half lives in the blood range from min to several h. Larger liposomes, such as MLVs and LUVs, are taken up rapidly by phagocytic cells of the reticuloendothelial system, but physiology of the circulatory system restrains the exit of such large species at most sites. They can exit only in places where large openings or pores exist in the capillary endothelium, such as the sinusoids of the liver or spleen. Thus, these organs are the predominate site of uptake. On the other hand, SUVs show a broader tissue distribution but still are sequestered highly in the liver and spleen. In general, this in vivo behavior limits the potential targeting of liposomes to only those organs and tissues accessible to their large size. These include the blood, liver, spleen, bone marrow, and lymphoid organs.

Targeting is generally not a limitation in terms of the present invention. However, should specific targeting be desired, methods are available for this to be accomplished. Antibodies may be used to bind to the liposome surface and to direct the antibody and its drug contents to specific antigenic receptors located on a particular cell-type surface. Carbohydrate determinants (glycoprotein or glycolipid cell-surface components that play a role in cell-cell recognition, interaction and adhesion) may also be used as recognition sites as they have potential in directing liposomes to particular cell types. Mostly, it is contemplated that intravenous injection of liposomal preparations would be used, but other routes of administration are also conceivable.

Alternatively, the invention provides for pharmaceutically-acceptable nanocapsule formulations of the compositions of the present invention. Nanocapsules can generally entrap compounds in a stable and reproducible way (Henry-Michelland et al., 1987; Quintanar-Guerrero et al., 1998; Douglas et al., 1987). To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention. Such particles may be are easily made, as described (Couvreur et al., 1980; 1988; zur Muhlen et al., 1998; Zambaux et al. 1998; Pinto-Alphandry et al., 1995 and U.S. Pat. No. 5,145,684, specifically incorporated herein by reference in its entirety).

### Vaccines

In certain preferred embodiments of the present invention, vaccines are provided. As shown in Example 5 below, the present fusion constructs elicit a strong cell-mediated immune response. The cell-mediated immune system responds to endogenous antigen presented by the MHC class I processing pathway. Cells can process foreign proteins found in the cell cytosol and display relevant peptide epitopes using this processing pathway (Harding, in Cellular Proteolytic Systems, pp. 163-180 (1994); Carbone & Bevan, in Fundamental Immunology, pp. 541-567 (Paul, ed., 1989); Townsend & Bodmer, Annu. Rev. Immunol. 7: 601-624 (1989)). The MHC class I processing pathway involves digestion of the antigen by the proteasome complex and transport of the resulting peptides into the endoplasmic reticulum, where they bind to nascent MHC class I molecules (Germain & Margulies, Annu. Rev. Immunol. 11: 403-450 (1993)). Cytotoxic T lymphocytes (CTLs) specifically recognize the foreign antigen displayed by the MHC class I molecules and lyse the antigen-presenting cells. A population of memory T cells is also established that can react to presentation of the specific antigen. The cellular immune system is thus primed to swiftly respond to an intracellular infection by a pathogenic organism such as a virus. The objective for a vaccine that stimulates the cell-mediated immune system is to deliver protein antigen to the cell cytosol for processing and subsequent presentation by MHC class I molecules.

The “MHC class I processing pathway” is an intracellular pathway that results in the binding of a peptide antigen ligand to an MHC class I molecule and the presentation of the antigen-MHC class I complex on the cell surface. First, cytoplasmic antigen is partially processed (through the action of proteasomes) and enters the ER as a complex with a transporter protein. In the ER, MHC class I molecules stably associate with the peptide antigen. The antigen-MHC class I complex then passes through the trans-Golgi network in a secretory vesicle to the cell surface. Functionally, processing of a peptide antigen through the MHC class I processing pathway can be identified with the use of lactacystin. Lactacystin is a specific proteasome inhibitor. Lactacystin inhibition of antigen presentation demonstrates that processing of the antigen is dependent on the function of the proteasome complex rather than an alternative processing pathway.

The present vaccines will generally comprise one or more pharmaceutical compositions, such as those discussed above, in combination with an immunostimulant. An immunostimulant may be any substance that enhances or potentiates an immune response (antibody and/or cell-mediated) to an exogenous antigen. Examples of immunostimulants include adjuvants, biodegradable microspheres (e.g., polylactic galactide) and liposomes (into which the compound is incorporated; see, e.g., Fullerton, U.S. Pat. No. 4,235,877). Vaccine preparation is generally described in, for example, Powell & Newman, eds., Vaccine Design (the subunit and adjuvant approach) (1995). Pharmaceutical compositions and vaccines within the scope of the present invention may also contain other compounds, which may be biologically active or inactive. For example, one or more immunogenic portions of other tumor antigens may be present, either incorporated into a fusion polypeptide or as a separate compound, within the composition or vaccine.

Illustrative vaccines may contain DNA encoding one or more of the polypeptides as described above, such that the polypeptide is generated in situ. As noted above, the DNA may be present within any of a variety of delivery systems known to those of ordinary skill in the art, including nucleic acid expression systems, bacteria and viral expression systems. Numerous gene delivery techniques are well known in the art, such as those described by Rolland, Crit. Rev. Therap. Drug Carrier Systems 15:143-198 (1998), and references cited therein. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface or secretes such an epitope. In a preferred embodiment, the DNA may be introduced using a viral expression system (e.g., vaccinia or other pox virus, retrovirus, or adenovirus), which may involve the use of a non-pathogenic (defective), replication competent virus. Suitable systems are disclosed, for example, in Fisher-Hoch et al., Proc. Natl. Acad. Sci. USA 86:317-321 (1989); Flexner et al., Ann. N.Y. Acad. Sci. 569:86-103 (1989); Flexner et al., Vaccine 8:17-21 (1990); U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques 6:616-627 (1988); Rosenfeld et al., Science 252:431-434 (1991); Kolls et al., Proc. Natl. Acad. Sci. USA 91:215-219 (1994); Kass-Eisler et al., Proc. Natl. Acad. Sci. USA 90:11498-11502 (1993); Guzman et al., Circulation 88:2838-2848 (1993); and Guzman et al., Cir. Res. 73:1202-1207 (1993). Techniques for incorporating DNA into such expression systems are well known to those of ordinary skill in the art. The DNA may also be “naked,” as described, for example, in Ulmer et al., Science 259:1745-1749 (1993) and reviewed by Cohen, Science 259:1691-1692 (1993). The uptake of naked DNA may be increased by coating the DNA onto biodegradable beads, which are efficiently transported into the cells. It will be apparent that a vaccine may comprise both a polynucleotide and a polypeptide component. Such vaccines may provide for an enhanced immune response.

It will be apparent that a vaccine may contain pharmaceutically acceptable salts of the polynucleotides and polypeptides provided herein. Such salts may be prepared from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts).

While any suitable carrier known to those of ordinary skill in the art may be employed in the vaccine compositions of this invention, the type of carrier will vary depending on the mode of administration. Compositions of the present invention may be formulated for any appropriate manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactate polyglycolate) may also be employed as carriers for the pharmaceutical compositions of this invention. Suitable biodegradable microspheres are disclosed, for example, in U.S. Pat. Nos. 4,897,268; 5,075,109; 5,928,647; 5,811,128; 5,820,883; 5,853,763; 5,814,344 and 5,942,252. One may also employ a carrier comprising the particulate-protein complexes described in U.S. Pat. No. 5,928,647, which are capable of inducing a class I-restricted cytotoxic T lymphocyte responses in a host.

Such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention may be formulated as a lyophilizate. Compounds may also be encapsulated within liposomes using well known technology.

Any of a variety of immunostimulants may be employed in the vaccines of this invention. For example, an adjuvant may be included. Most adjuvants contain a substance designed to protect the antigen from rapid catabolism, such as aluminum hydroxide or mineral oil, and a stimulator of immune responses, such as lipid A, Bortadella pertussis or Mycobacterium species or Mycobacterium derived proteins. For example, delipidated, deglycolipidated M. vaccae (“pVac”) can be used. In another embodiment, BCG is used as an adjuvant. In addition, the vaccine can be administered to a subject previously exposed to BCG. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 and derivatives thereof (SmithKline Beecham, Philadelphia, Pa.); CWS, TDM, Leif, aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, may also be used as adjuvants.

Within the vaccines provided herein, the adjuvant composition is preferably designed to induce an immune response predominantly of the Th1 type. High levels of Th1-type cytokines (e.g., IFN-γ, TNFα, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann & Coffman, Ann. Rev. Immunol. 7:145-173 (1989).

Preferred adjuvants for use in eliciting a predominantly Th1-type response include, for example, a combination of monophosphoryl lipid A, preferably 3-de-O-acylated monophosphoryl lipid A (3D-MPL), together with an aluminum salt. MPL adjuvants are available from Corixa Corporation (Seattle, Wash.; see U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352 (1996). Another preferred adjuvant comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins. Other preferred formulations include more than one saponin in the adjuvant combinations of the present invention, for example combinations of at least two of the following group comprising QS21, QS7, Quil A, β-escin, or digitonin.

Alternatively, the saponin formulations may be combined with vaccine vehicles composed of chitosan or other polycationic polymers, polylactide and polylactide-co-glycolide particles, poly-N-acetyl glucosamine-based polymer matrix, particles composed of polysaccharides or chemically modified polysaccharides, liposomes and lipid-based particles, particles composed of glycerol monoesters, etc. The saponins may also be formulated in the presence of cholesterol to form particulate structures such as liposomes or ISCOMs. Furthermore, the saponins may be formulated together with a polyoxyethylene ether or ester, in either a non-particulate solution or suspension, or in a particulate structure such as a paucilamelar liposome or ISCOM. The saponins may also be formulated with excipients such as Carbopol^{R} to increase viscosity, or may be formulated in a dry powder form with a powder excipient such as lactose.

In one preferred embodiment, the adjuvant system includes the combination of a monophosphoryl lipid A and a saponin derivative, such as the combination of QS21 and 3D-MPL® adjuvant, as described in WO 94/00153, or a less reactogenic composition where the QS21 is quenched with cholesterol, as described in WO 96/33739. Other preferred formulations comprise an oil-in-water emulsion and tocopherol. Another particularly preferred adjuvant formulation employing QS21, 3D-MPL® adjuvant and tocopherol in an oil-in-water emulsion is described in WO 95/17210.

Another enhanced adjuvant system involves the combination of a CpG-containing oligonucleotide and a saponin derivative particularly the combination of CpG and QS21 as disclosed in WO 00/09159. Preferably the formulation additionally comprises an oil in water emulsion and tocopherol.

Other preferred adjuvants include Montanide ISA 720 (Seppic, France), SAF (Chiron, Calif., United States), ISCOMS (CSL), MF-59 (Chiron), the SBAS series of adjuvants (e.g., SBAS-2, AS2′, AS2,″ SBAS-4, or SBAS6, available from SmithKline Beecham, Rixensart, Belgium), Detox (Corixa, Hamilton, Mont.), RC-529 (Corixa, Hamilton, Mont.) and other aminoalkyl glucosaminide 4-phosphates (AGPs), such as those described in pending U.S. patent application Ser. Nos. 08/853,826 and 09/074,720, the disclosures of which are incorporated herein by reference in their entireties, and polyoxyethylene ether adjuvants such as those described in WO 99/52549A1.

Other preferred adjuvants include adjuvant molecules of the general formula (I): HO(CH₂CH₂O)ₙ—A—R, wherein, n is 1-50, A is a bond or —C(O)—, R is C₁₋₅₀ alkyl or Phenyl C₁₋₅₀ alkyl.

One embodiment of the present invention consists of a vaccine formulation comprising a polyoxyethylene ether of general formula (I), wherein n is between 1 and 50, preferably 4-24, most preferably 9; the R component is CI-₅₀, preferably C₄-C₂₀ alkyl and most preferably C₁₂ alkyl, and A is a bond. The concentration of the polyoxyethylene ethers should be in the range 0.1-20%, preferably from 0.1-10%, and most preferably in the range 0.1-1%. Preferred polyoxyethylene ethers are selected from the following group: polyoxyethylene-9-lauryl ether, polyoxyethylene-9-steoryl ether, polyoxyethylene-8-steoryl ether, polyoxyethylene-4-lauryl ether, polyoxyethylene-35-lauryl ether, and polyoxyethylene-23-lauryl ether. Polyoxyethylene ethers such as polyoxyethylene lauryl ether are described in the Merck index (12^{th} edition: entry 7717). These adjuvant molecules are described in WO 99/52549.

The polyoxyethylene ether according to the general formula (I) above may, if desired, be combined with another adjuvant. For example, a preferred adjuvant combination is preferably with CpG as described in the pending UK patent application GB 9820956.2.

Any vaccine provided herein may be prepared using well known methods that result in a combination of antigen, immune response enhancer and a suitable carrier or excipient. The compositions described herein may be administered as part of a sustained release formulation (i.e., a formulation such as a capsule, sponge or gel (composed of polysaccharides, for example) that effects a slow release of compound following administration). Such formulations may generally be prepared using well known technology (see, e.g., Coombes et al., Vaccine 14:1429-1438 (1996)) and administered by, for example, oral, rectal or subcutaneous implantation, or by implantation at the desired target site. Sustained-release formulations may contain a polypeptide, polynucleotide or antibody dispersed in a carrier matrix and/or contained within a reservoir surrounded by a rate controlling membrane.

Carriers for use within such formulations are biocompatible, and may also be biodegradable; preferably the formulation provides a relatively constant level of active component release. Such carriers include microparticles of poly(lactide-co-glycolide), polyacrylate, latex, starch, cellulose, dextran and the like. Other delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see, e.g., U.S. Pat. No. 5,151,254 and PCT applications WO 94/20078, WO/94/23701 and WO 96/06638). The amount of active compound contained within a sustained release formulation depends upon the site of implantation, the rate and expected duration of release and the nature of the condition to be treated or prevented.

Any of a variety of delivery vehicles may be employed within pharmaceutical compositions and vaccines to facilitate production of an antigen-specific immune response that targets tumor cells. Delivery vehicles include antigen presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have anti-tumor effects per se and/or to be immunologically compatible with the receiver (i.e., matched HLA haplotype). APCs may generally be isolated from any of a variety of biological fluids and organs, including tumor and peritumoral tissues, and may be autologous, allogeneic, syngeneic or xenogeneic cells.

Certain preferred embodiments of the present invention use dendritic cells or progenitors thereof as antigen-presenting cells. Dendritic cells are highly potent APCs (Banchereau & Steinman, Nature 392:245-251 (1998)) and have been shown to be effective as a physiological adjuvant for eliciting prophylactic or therapeutic antitumor immunity (see Timmerman & Levy, Ann. Rev. Med. 50:507-529 (1999)). In general, dendritic cells may be identified based on their typical shape (stellate in situ, with marked cytoplasmic processes (dendrites) visible in vitro), their ability to take up, process and present antigens with high efficiency and their ability to activate naive T cell responses. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within a vaccine (see Zitvogel et al., Nature Med. 4:594-600 (1998)).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, LPS, flt3 ligand and/or other compound(s) that induce differentiation, maturation and proliferation of dendritic cells.

Dendritic cells are conveniently categorized as “immature” and “mature” cells, which allows a simple way to discriminate between two well characterized phenotypes. However, this nomenclature should not be construed to exclude all possible intermediate stages of differentiation. Immature dendritic cells are characterized as APC with a high capacity for antigen uptake and processing, which correlates with the high expression of Fcγ receptor and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1BB).

APCs may generally be transfected with a polynucleotide encoding a protein (or portion or other variant thereof) such that the polypeptide, or an immunogenic portion thereof, is expressed on the cell surface. Such transfection may take place ex vivo, and a composition or vaccine comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., Immunology and Cell Biology 75:456-460 (1997). Antigen loading of dendritic cells may be achieved by incubating dendritic cells or progenitor cells with the polypeptide, DNA (naked or within a plasmid vector) or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule). Alternatively, a dendritic cell may be pulsed with a non-conjugated immunological partner, separately or in the presence of the polypeptide.

Vaccines and pharmaceutical compositions may be presented in unit-dose or multi-dose containers, such as sealed ampoules or vials. Such containers are preferably hermetically sealed to preserve sterility of the formulation until use. In general, formulations may be stored as suspensions, solutions or emulsions in oily or aqueous vehicles. Alternatively, a vaccine or pharmaceutical composition may be stored in a freeze-dried condition requiring only the addition of a sterile liquid carrier immediately prior to use.

### Diagnostic Kits

The present invention further provides kits for use within any of the above diagnostic methods. Such kits typically comprise two or more components necessary for performing a diagnostic assay. Components may be compounds, reagents, containers and/or equipment. For example, one container within a kit may contain a monoclonal antibody or fragment thereof that specifically binds to a protein. Such antibodies or fragments may be provided attached to a support material, as described above. One or more additional containers may enclose elements, such as reagents or buffers, to be used in the assay. Such kits may also, or alternatively, contain a detection reagent as described above that contains a reporter group suitable for direct or indirect detection of antibody binding.

Alternatively, a kit may be designed to detect the level of mRNA encoding a protein in a biological sample. Such kits generally comprise at least one oligonucleotide probe or primer, as described above, that hybridizes to a polynucleotide encoding a protein. Such an oligonucleotide may be used, for example, within a PCR or hybridization assay. Additional components that may be present within such kits include a second oligonucleotide and/or a diagnostic reagent or container to facilitate the detection of a polynucleotide encoding a protein of the invention.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

### EXAMPLES

The following examples are offered to illustrate, but not to limit the claimed invention.

### Example 1

### DNA Constructs Comprising the Leishmania Gene TSA at the N-terminus and Linked with the TB Antigens, MTB8.4 or MTB12

The following DNA constructs comprising the Leishmania gene TSA (also referred to as “MAPS”) at the N-terminus linked with the TB antigens (DPV & DPAS; aka Mtb8.4 and Mtb12) were produced. The DNA (genetic fusion construct) was cloned into the eukaryotic DNA expression vector (pcDNA3) for transfection and DNA vaccine studies. Specifically, the following nucleic acid constructs were made: 1) MAPS-DPV pET (same as TSA-Mtb8.4; SEQ ID NO:50); and 2) MAPS-DPAS pET (same as TSA-Mtb12; SEQ ID NO:52). These constructs are for protein expression for the generation of recombinant antigens. Also made are the following constructs: 3) MAPS-DPV pcDNA (same as TSA-Mtb8.4; SEQ ID NO:54); and 4) MAPS-DPAS pcDNA (same as TSA-Mtb12; SEQ ID NO:55). These constructs are useful as DNA vaccine constructs. The protein sequences are also provided: 1) MAPS-DPV pET.pro (SEQ ID NO:51); and 2)MAPS-DPAS-pET.pro (SEQ ID NO:53). These sequences are recombinant proteins expressed from the corresponding nucleotide sequences described above with 6xHis residues for purification.

### Example 2

### DNA Constructs Comprising the Leishmania Gene TSA at the N-terminus and Linked with the Codon Optimized TB Antigens, MTB8.4 or MTB12

Highly expressed Leishmania TSA gene (also referred to as MAP) was fused with the codon optimized Mtb antigens DPV-AC (Mtb8.4) and DPAS-AC (Mtb12). The MAPS fusion vector was constructed in the plasmid pcDNA3.1 (Invitrogen). The MAPS gene was amplified by PCR using primers that removed the MAPS termination codon and introduced an EcoRI cloning site at the 3′ end of the coding sequence.

DPV-AC (altered codon) and DPAS-AC were constructed as follows. The coding sequences for the M. tuberculosis (Mtb) antigens DPV and DPAS were reconstructed to bias the codon usage toward that of mammalian, in this case murine, genes. To determine which codons of DPV and DPAS to alter a comparison was made of the Mtb and Mus musculus codon usage tables (www.kazusa.or.jp/codon/), which are based on the analysis of 1432158 and 4168443 codons, respectively. DPV and DPAS codons that are used infrequently in murine coding sequences were changed to the most frequently used Mus musculus codons. In order to avoid overrepresenting altered codons, in cases where a particular DPV or DPAS codon was repeatedly changed from low frequency Mtb usage to high frequency Mus musculus usage, the next most frequently used Mus musculus codon was substituted. This resulted in hypothetical altered codon DPV and DPAS coding sequences (DPV-AC and DPAS-AC) with a Mus musculus codon bias. DPV-AC and DPAS-AC were constructed using a series of codon biased sense and antisense oligonucleotides.

For DPV-AC 8 pairs of sense and antisense oligonucleotides, ranging in length from 28 bp to 40 bp were designed. Oligonucleotide pair 1, consisting of oligonucleotides 1 and 2, incorporated a HindIII site for subsequent cloning into vector JA4304, a Kozak consensus sequence and an ATG start codon 5′ of the DPV-AC coding sequence. Oligonucleotide pair 8 included a NheI site for cloning into JA4304 3′ of the TAA stop codon. Specifics for the individual DPV-AC oligonucleotides are as follows; DPV-AC oligo # position comments 1 sense (s) −19/18 D of DPV is +1 and G of ATG is −1.2 antisense (as) −19/9 pair 1; 9 bp sense overhang. 3 s 19/56 4 as 10/46 pair 2; 9 bp as and 10 bp s overhang. 5s57/91 6as47/82 pair3; 10 bp as and 9 bp s overhang. 7 s 92/1278as 83/118 pair 4; 9 bp as and 9 bp s overhang. 9 s 128/162 10 as 119/153 pair 5; 9 bp as and 9 bp s overhang. 11 s 163/199 12 as 154/190 pair 6; 9 bp as and 9 bp s overhang. 13 s 200/229 14 as 191/219 pair 7; 9 bp as and 10 bp s overhang. 15 s 230/249 includes 10 bp 3′ of TAA containing NheI site. 16 as 220/249 pair 8; 10 bp as overhang.

For DPAS-AC 9 pairs of sense and antisense oligonucleotides, ranging in length from 33 bp to 47 bp were designed. Oligonucleotide pair 1, consisting of oligonucleotides 1 and 2, incorporated a HindIII site for subsequent cloning into vector JA4304, a Kozak consensus sequence and an ATG start codon 5′ of the DPAS-AC coding sequence. Oligonucleotide pair 9 included a NheI site for cloning into JA4304 3′ of the TGA stop codon. Specifics for the individual DPAS-AC oligonucleotides are as follows; DPAS-AC oligo # position comments 1 sense (s) −19/24 D of DPAS is +1 and G of ATG is −1. 2 antisense (as) −19/14 pair 1; 10 bp sense overhang. 3 s 25/69 4 as 15/58 pair 2; 10 bp as and 11 bps overhang. 5 s 70/115 6 as 59/106 pair 3; 11 bp as and 9 bps overhang. 7 s 116/160 8 as 107/150 pair 4; 9 bp as and 10 bps overhang. 9 s 161/206 10 as 151/195 pair 5; 10 bp as and 11 bps overhang. 11 s 207/251 12 as 196/241 pair 6; 11 bp as and 10 bp s overhang. 13 s 252/295 14 as 242/284 pair 7; 10 bp as and 11 bps overhang. 15 s 296/340 16 as 285/327 pair 8; 11 bp as and 13 bps overhang. 17 s 341/363 includes 10 bp 3′ of TGA containing NheI site. 18 as 328/363 pair 9; 13 bp as overhang.

All DPV-AC and DPAS-AC oligonucleotides were obtained from Gibco-BRL. All oligonucleotides were reconstituted at 0.5 nmole/μl (˜6-5 μg/μl) with H₂O. Pairs of oligonucleotides were combined (1 with 2, 3 with 4, etc., 17 with 18) in 20 μl annealing reactions containing 100 pmole/μl of each oligonucleotide in 10 mM Tris-HCl, pH 7.5, 0.1M NaCl, 10 mM EDTA. DPV-AC and DPAS-AC oligonucleotide pairs were placed at 65° C. for 10 minutes and 94° C. for 3 minutes, respectively, and allowed to anneal slowly at room temperature (25° C.) for 90 minutes. DPV-AC and DPAS-AC oligonucleotide pairs were then diluted 20- and 10-fold with H₂O to 5 pmole/μl (˜120 ng/μl) and 10 pmole/μl (˜240 ng/μl), respectively.

Next, 5 pmole and 10 pmole, respectively, of the individual DPV-AC and DPAS-AC oligonucleotide pairs were kinased with 10U of T4 polynucleotide kinase (Gibco-BRL) and 1 mM ATP for 10 minutes at 37° C. All DPV-AC or DPAS-AC oligonucleotide pairs were combined (˜1 μg and 2 μg total DNA, respectively), placed at 65° C. for 15 minutes to inactivate the kinase and then allowed to cool to room temperature for 30 minutes to anneal the overhangs. Ligations were performed by adding T4 DNA ligase reaction buffer to 1× to the annealed DPV-AC and DPAS-AC oligonucleotide pairs, adding 25U to 30U T4 DNA ligase (Gibco-BRL) and allowing the reactions to proceed for 3 hours at room temperature (25° C.). Impurities were removed from the DPV-AC and DPAS-AC DNA using a Qiaquick gel extraction kit as per the manufacturers instructions (Qiagen).

The DPV-AC and DPAS-AC DNA was digested with HindIII and NheI, electrophoresed through 1.5% agarose and regions corresponding to the expected size products for DPV-AC and DPAS-AC (268 bp and 382 bp) were excised from the gel, isolated using a Qiaquick gel extraction kit and directionally cloned into JA4304. To confirm that DPV-AC and DPAS-AC in JA4304 were as expected the sense and antisense strands were completely sequenced. The codon optimized DPAS-AC and DPV-AC DNA sequences are shown in SEQ ID NOS:66 and 67, respectively.

For fusion to MAPS, DPV-AC and DPAS-AC were PCR amplified with primers containing EcoRI restriction sites. The 5′ primer EcoRI site allowed for DPV-AC and DPAS-AC to be cloned in-frame to the 3′ end of MAPS, while the 3′ primer EcoRI site was placed downstream of a termination codon. Following PCR amplification, DPV-AC and DPAS-AC were gel purified, digested with EcoRI and cloned into EcoRI digested MAPS fusion vector. The resulting pcDNA3.1-based MAPS-DPV-AC (TSA-Mtb8.4-AC; SEQ ID NO:56) and MAPS-DPAS-AC (TSA-Mtb12-AC; SEQ ID NO:58 ) fusion plasmids were verified by sequence analysis. The protein sequences of codon optimized MAPS-DPV-AC and MAPS-DPAS-AC are shown in SEQ ID NOS: 57 and 59, respectively.

### Example 3

### Protein Expression Levels of MAP-DPV-AC, MAP-DPAS-AC, MAP-DPV and MAP-DPAS, DPV and DPAS

The protein expression levels of the MAPS-DPV-AC and MAPS-DPAS-AC were measured following their transfection into human embryonic kidney (HEK) 293 cells. These protein expression levels were compared to similarly transfected constructs encoding DPV, DPV-AC, MAPS-DPV and DPAS, DPAS-AC, MAPS-DPAS and empty JA4304. Briefly, about 2×10⁵ (˜30% confluent) HEK 293 cells in DMEM/10% FBS were plated onto 35 mm culture dishes. DNA to be tested was brought to 1 g in 10 L H₂O (0.1 g/l). The FuGene 6 transfection reagent was prepared, and was added to the DNA. The FuGene 6/DNA mix was used to transfect the HEK 293 cells according to the manufacturer's instructions (Boehringer Mannheim). The HEK 293 cells were incubated for 48 to 72 hours at 37° C. and harvested. The cells were collected by centrifugation for 7 minutes at 1.2 K rpm, resuspended in 250 L of 0.1M Tris, pH8, 4% SDS, 20% glycerol. After sonication for 30 seconds, the lysate protein concentration was determined by BCA assay (Pierce). 10 g of total protein was loaded per well, subjected to SDS PAGE and blotted to nitrocellulose. Rabbit polyclonal antibodies raised against DPV, DPAS and MAPS (1:10K dilution) followed by a donkey anti-rabbit HRP conjugated secondary antibody (1:10K dilution) and ECL (Amersham Pharmacia Biotech) were used to detect the expression of these proteins. The lysates were also analyzed by SDS PAGE and coomassie staining.

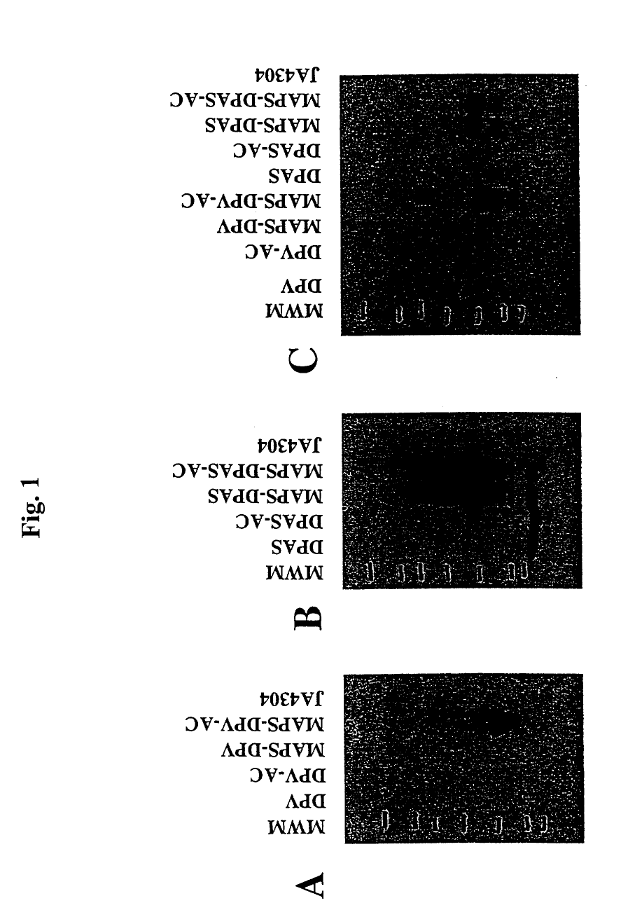

FIGS. 1A-C illustrate Western blots of various DNA construct expression in HEK293 cells. As shown in the figures, data indicate that fusion of codon optimized DPV to MAPS (MAPS-DPV-AC) and fusion of DPAS to MAPS (MAPS-DPAS) significantly boosts the expression of these antigens in eukaryotic cells. DPV is normally detectable on a Western blot only after a lengthy exposure and is never observed on a coomassie stained gel. The same holds true for DPV-AC and MAPS-DPV. Following fusion of MAPS to codon optimized DPV-AC, however, DPV expression is readily observed on a Western blot and is visible on SDS PAGE by coomassie staining. It is likely that the combination of DPV codon optimization and fusion to MAPS is required to achieve high level DPV expression, since neither DPV-AC nor MAPS-DPV (non-codon optimized) shows an increase in expression compared to the original DPV. In contrast to DPV, DPAS can be seen on Western blots and coomassie stained gels. When compared to DPAS and DPAS-AC, however, the MAPS-DPAS fusion results in a significant increase in the total amount of DPAS antibody reactive protein produced in the HEK 293 cells. This increase in protein expression appears dependent on fusion to MAPS, as the expression levels of DPAS-AC and MAPS-DPAS-AC are equivalent to the expression levels of DPAS and MAPS-DPAS, respectively.

### Example 4

### Truncated MAPS Constructs—MAPS(N5)/DPV-AC and MAPS(N10)/DPV-AC DNA and Their Expression

To minimize the immune response to MAPS while maintaining a high level of DPV expression, two truncated MAPS encoding the first five and ten amino acids of MAPS were fused to the DPV-AC gene. The constructs were evaluated for their ability to drive high level DPV expression in a DNA vaccine format. MAPS(N5)/DPV-AC (SEQ ID NOS:60 and 61) and MAPS(N10)/DPV-AC (SEQ ID NOS:62 and 63), hybrid sequences encoding DPV-AC downstream of the first five and ten amino of MAPS, respectively, were constructed in JA4304 as follows. The hybrid sequences were generated using the megaprimer PCR method. Primers MAPSN5-DPV-AC (5′ GATAAAGCTTGCAATCATGTCCTGCGGT AACGACCCCGTGGACGCCGTGAT 3′) and MAPSN10-DPV-AC (5′GATAAAGCTT GCAATCATGTCCTGCGGTAACGCCAAGATCAACTCTGACCCCGTGGACGCCGTGA T 3′), which include a HindIII restriction site, a kozak sequence, the coding sequence for the first five and ten amino acids of MAPS in frame with the sequence of DPV-AC, were used with primer DPV-AC-NheI-R (5′ GATAGCTAGCTTAGTAGTTGTTGCAGGAGCCG 3′) to amplify MAPS(N5)/DPV-AC and MAPS(N10)/DPV-AC. The PCR products were gel isolated, digested with HindIII and NheI, and cloned into HindIII/NheI cut JA4304. The inserts were fully sequenced to confirm that intact fusions were generated.

The protein expression levels of the MAPS(N5)/DPV-AC and MAPS(N10)/DPV-AC DNA vaccines were measured following their transfection into human embryonic kidney (HEK) 293 cells and compared to similarly transfected constructs encoding DPV, DPV-AC, MAPS, MAPS/DPV, MAPS/DPV-AC and empty JA4304. Briefly, about 2×10⁵ (˜30% confluent) HEK 293 cells in DMEM/10% FBS were plated onto 35 mm culture dishes. DNA to be tested was brought to 1 g in 10 L H₂O (0.1 g/l). The FuGene 6 transfection reagent was prepared, and was added to the DNA. The FuGene 6/DNA mix was used to transfect the HEK 293 cells according to the manufacturer's instructions (Boehringer Mannheim). The HEK 293 cells were incubated for 48 to 72 hours at 37° C. and harvested. The cells were collected by centrifugation for 10 minutes at 1.2 K rpm, resuspended in 250 L PBS and lysed by the addition of 250 L of 0.1M Tris, pH 8, 4% SDS, 20% glycerol. After sonication for 30 seconds, the lysate protein concentration was determined by BCA assay (Pierce). 10 g of total protein was loaded per well, subjected to SDS PAGE and blotted to nitrocellulose. Rabbit polyclonal antibodies raised against DPV and MAPS (1:10K dilution) followed by a donkey anti-rabbit HRP conjugated secondary antibody (1:10K dilution) and ECL (Amersham Pharmacia Biotech) were used to detect the expression of these proteins. The lysates were also analyzed by SDS PAGE and coomassie staining.

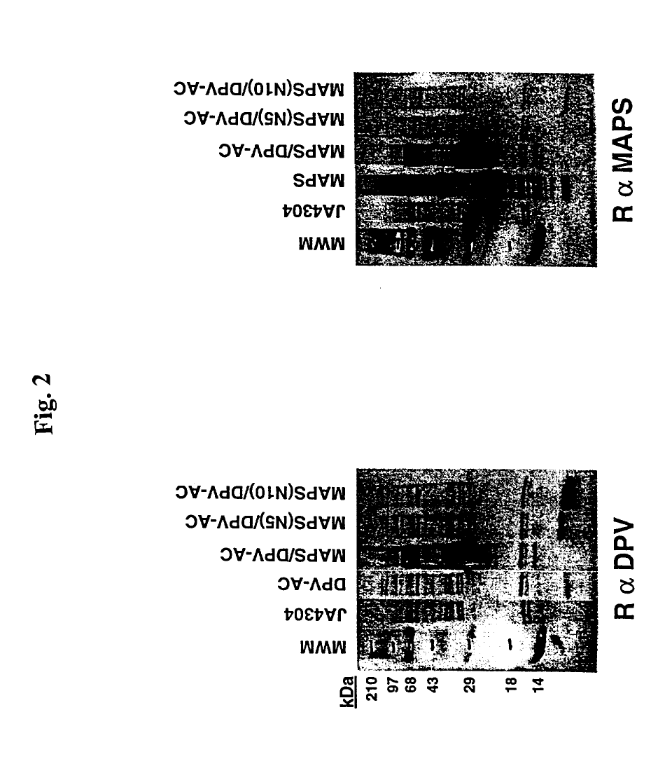

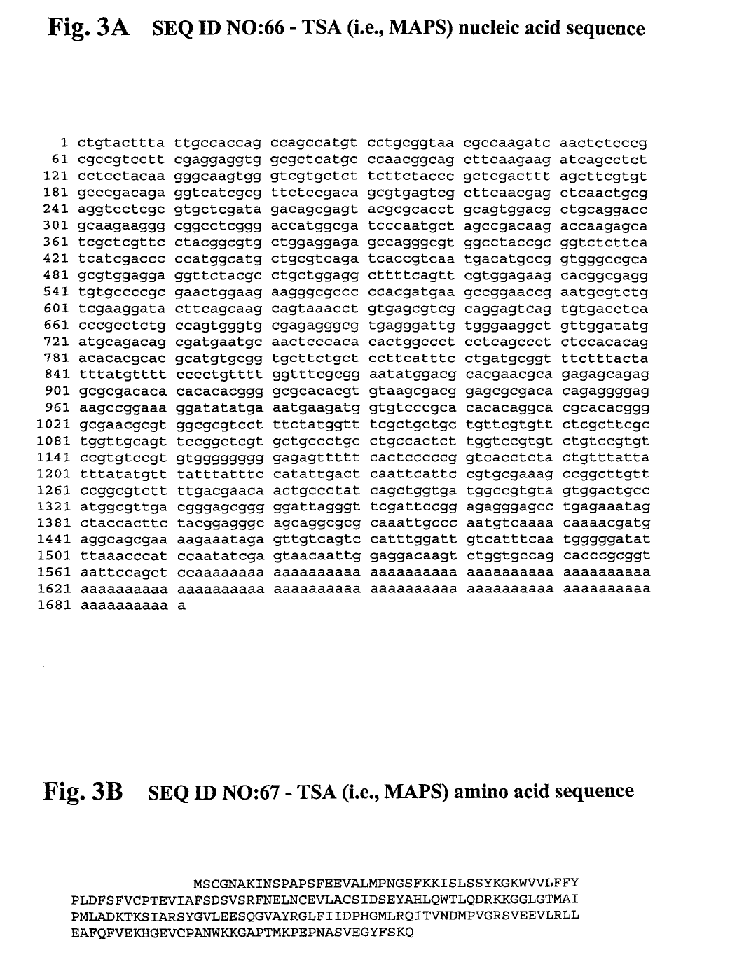

FIG. 2 illustrates Western blots of various DNA construct expression in HEK293 cells. As shown in the figure, data indicate that fusion of the codon optimized DPV gene to sequences encoding the first five (MAPS(N5)/DPV-AC) and, in particular, the first ten (MAPS(N10)/DPV-AC) amino acids of MAPS significantly boosts the expression of these antigens in eukaryotic cells. DPV is normally detectable on a Western blot only after a lengthy exposure and is never observed on a coomassie stained gel. The same holds true for DPV-AC. Following the transfection of MAPS(N10)/DPV-AC into HEK 293 cells DPV expression is readily observed on a Western blot using rabbit anti DPV serum. DPV expression is also elevated for MAPS(N5)/DPV-AC, although the level attained is much less than that seen for MAPS(N10)/DPV-AC. When the relative expression levels are compared, it is clear that fusions of DPV to full-length MAPS (MAPS/DPV and MAPS/DPV-AC) are most highly expressed, followed by the MAPS(N10)/DPV-AC fusion and then MAPS(N5)/DPV-AC.

It is important to note that all of the fusion constructs significantly elevate DPV expression compared to DPV alone. Interestingly, while full-length MAPS and its DPV hybrids react strongly with rabbit anti MAPS serum, MAPS(N10)/DPV-AC and MAPS(N5)/DPV-AC reactivity is barely detectable. This suggests that the first ten amino acids of MAPS do not contain an antibody producing epitope. In summary, while MAPS(N10)/DPV-AC and MAPS(N5)/DPV-AC do not attain the extremely high levels of DPV expression observed for the fusion of full-length MAPS to DPV, they produce tremendous increases in the level of DPV expression, while generating virtually no MAPS antibody response.

### Example 5

### Mouse Vaccination

The immune responses produced in mice by the various hybrid MAPS/DPV, MAPS and DPV DNA vaccines were compared. Groups of eight mice were immunized with 100 μg of the various MAPS/DPV DNA vaccines at three week intervals. Following the immunizations, three of the mice were analyzed for a number of immune responses, including the production of IFN-γ, type-specific antibodies and cytotoxic T lymphocytes (CTL). Splenocytes from mice immunized with MAPS produced IFN-γ, only following restimulation with MAPS protein. In contrast, the splenocytes from MAPS/DPV immunized animals produced IFN-γ in response to MAPS and DPV recombinant proteins. Similarly, while MAPS immunization produced IgG1 and IgG2a type-specific antibodies only to MAPS, immunization with MAPS/DPV resulted in the production of antibodies to both MAPS and DPV. Intriguingly, DPV DNA immunization alone produced no IFN-γ, or specific antibody, underscoring the ability of MAPS to increase DPV expression and subsequent immune responses.

Finally, the constructs were compared for their ability to generate DPV-specific CTL. DPV is known to generate CTL responses and did so in this experiment. A comparable level of CTL resulted from immunizations with MAPS(N10)/DPV. However, MAPS/DPV caused significantly higher levels of CTL production, suggesting that the increased expression of DPV driven by MAPS was responsible. In summary, these results demonstrate that the fusion of MAPS to DPV does not diminish the ability of either antigen to stimulate the production of IFN-γ, antigen-specific antibodies or of DPV to mount a CTL response. Most notably, the high level expression of DPV results in an increased IFN-γ, type-specific antibody and CTL responses, suggesting that the MAPS/DPV hybrid DNA vaccine may prove more effective at protecting against tuberculosis infection.

### Example 6

### DNA constructs Comprising MTB72F/MAPS r95f

MTB72F has been shown to protect against TB challenge in three animal models (mouse, guinea pig and monkeys). Several other antigens shown to elicit T cell responses in healthy PPD positive donors are potential vaccine candidates. To improve on the protective efficacy of MTB72F, genetic fusion constructs with MTB72F as backbone were constructed. New MTB72F fusions MTB72F (a 72 kDa poly-protein fusion construct comprising Ra12-TbH9-Ra35) was used as a backbone to add several of other candidate antigens. These include, e.g., MTB72F/MAPS r95f. The nucleotide and polypeptide sequences of this construct are shown as SEQ ID NOS:64 and 65, respectively.

### Example 7

### DPV-AC-MAPs Fusion Protein with DPV-AC Fused Upstream of MAPS

A DPV-AC/MAPS DNA vaccine in JA4304 has been constructed for comparison to MAPS/DPV DNA vaccine in an effort to understand the mechanism underlying the ability of MAPS to boost DPV protein expression and DPV-specific immune responses.

MAPS (TSA) is a Leishmania major protein that is expressed at a high level from the eukaryotic expression vector JA4304. When DPV was fused downstream of MAPS (MAPS/DPV) in the DNA vaccine format, a greater amount of DPV protein was produced with a concomitant increase in the DPV-specific immune response, as assayed by a number of methods.

In an effort to understand the biochemical and immunological mechanism(s) underlying the increases in total DPV protein and DPV-specific immune responses following immunization of mice the fusion DPV-AC/MAPS was constructed (SEQ ID NOS: 75 and 76). DPV-AC/MAPS was constructed by fusing in frame the codon-optimized version of DPV (DPV-AC) upstream of MAPS, using the following PCR-based strategy. The DPV-AC gene was PCR amplified using the oligonucleotides DPV-AC-HindIII-sense-I (which adds a restriction site and kozak sequence) and DPV-AC antisense (which removes the DPV stop codon). MAPS was PCR amplified using the oligonucleotides DPV-AC/MAPS fusion (which is a hybrid oligo that anneals to the 3′-end of the DPV-AC sequence and the 5′-end of MAPS) and MAPS-3-BamHI (which adds a restriction site downstream of the MAPS stop codon). The two PCR products, DPV-AC and MAPS with the DPV-AC sequence leader, were then mixed, annealed and subjected to an additional round of PCR using the outside oligonucleotides, DPV-AC-HindIII-sense-I and MAPS-3-BamHI, to generate the DPV-AC/MAPS fusion. This DNA was directionally cloned into HindIII and BamHI cut JA4304, and purified on a large scale by Qiagen endo-free DNA giga-prep.

The DPV-AC/MAPS fusion DNA has been compared to the DPV, MAPS and MAPS/DPV DNA vaccine constructs following transfection into HEK 293T cells for protein expression levels by Western blot and relative transcript levels by RT-PCR. Western blotting of HEK 293T cell lysates demonstrated that DPV-AC/MAPS plasmid produces an amount of DPV protein similar to that of MAPS/DPV and greater than that produced by the DPV plasmid alone. This result indicates that, when fused either upstream or downstream of DPV, MAPS can increase the level of DPV protein expression. In fact, the levels of DPV-AC/MAPS, MAPS/DPV and MAPS protein are produced at sufficient levels to be visible by coomassie steining. The level of MAPS protein produced by the DPV-AC/MAPS plasmid is approximately equal to that of MAPS plasmid alone and revealed that the amount of DPV produced has been increased to the normal level of MAPS. RT-PCR analysis demonstrated that there was no difference in the amount of DPV-specific transcript in HEK 293T cells transfected with the DPV, MAPS/DPV or DPV-AC/MAPS plasmids. Without being bound by theory, this result suggests that MAPS may be increasing the amount of DPV protein by stabilizing DPV within the cell and preventing its rapid degradation.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. An expression cassette comprising a recombinant nucleic acid molecule encoding a fusion polypeptide, the recombinant nucleic acid molecule comprising a heterologous polynucleotide sequence encoding an antigen or an antigenic fragment, and a Leishmania polynucleotide sequence encoding a polypeptide or a fragment thereof, wherein the Leishmania polynucleotide is selected from the group consisting of TSA polynucleotide, LeIF polynucleotide, M15 polynucleotide, and 6H polynucleotide, and wherein the recombinant nucleic acid molecule is operably linked to a eukaryotic promoter.

2. The expression cassette of claim 1, wherein the Leishmania polynucleotide sequence is the TSA polynucleotide.

3. The expression cassette of claim 1, wherein the Leishmania polynucleotide sequence is the TSA polynucleotide.

4. The expression cassette of claim 2, wherein the TSA polynucleotide is a N-terminal fragment of the Leishmania thiol-specific-antioxidant gene.

5. The expression cassette of claim 4, wherein the N-terminal fragment of the Leishmania thiol-specific-antioxidant gene comprises about 30 or less nucleotides.

6. The expression cassette of claim 1, wherein the heterologous polynucleotide sequence encodes a malaria antigen, a cancer antigen, a viral antigen or a bacterial antigen.

7. The expression cassette of claim 6, wherein the heterologous polynucleotide sequence is a Mycobacterium polynucleotide sequence encoding an antigen or antigenic fragment thereof from a Mycobacterium species of the tuberculosis complex.

8. The expression cassette of claim 7, wherein the Mycobacterium species is Mycobacterium tuberculosis.

9. The expression cassette of claim 7, wherein the Mycobacterium polynucleotide sequence encodes for MTB8.4 antigen, MTB9.8 antigen, MTB9.9 antigen, MTB12 antigen, MTB32A antigen, MTB40 antigen, MTB41 antigen, TbH9 antigen, Ra35 antigen, Ra12 antigen, 38-1 antigen, TbRa3 antigen, 38 kD antigen, DPEP antigen, TbH4 antigen, DPPD antigen, MTB82 antigen, Erd14 antigen, ESAT-6 antigen, MTB85b complex antigen, or an immunogenic fragment thereof.

10. The expression cassette of claim 7, wherein the Mycobacterium polynucleotide sequence encodes for MTB59F antigen, MTB72F antigen, MTB3 1F antigen, MTB71F antigen, MTB103F, or an immunogenic fragment thereof.

11. The expression cassette of claim 2, wherein the heterologous polynucleotide sequence encodes for MTB8.4 antigen or MTB12 antigen.

12. The expression cassette of claim 1, wherein the Leishmania polynucleotide sequence is located 5′ to the heterologous polynucleotide sequence.

13. The expression cassette of claim 1, wherein the Leishmania polynucleotide sequence is located 3′ to the heterologous polynucleotide sequence.

14. The expression cassette of claim 1, wherein the heterologous polynucleotide sequence is codon optimized for expression in eukaryotic cells.

15. The expression cassette of claim 14, wherein the Leishmania polynucleotide sequence is the TSA polynucleotide.

16. The expression cassette of claim 14, wherein the heterologous polynucleotide sequence encodes a malaria antigen, a cancer antigen, a viral antigen or a bacterial antigen.

17. The expression cassette of claim 16, wherein the heterologous polynucleotide sequence is a Mycobacterium polynucleotide sequence encoding an antigen or antigenic fragment thereof from a Mycobacterium species of the tuberculosis complex.

18. The expression cassette of claim 17, wherein the Mycobacterium polynucleotide sequence encodes for MTB8.4 antigen, MTB9.8 antigen, MTB9.9 antigen, MTB12 antigen, MTB32A antigen, MTB40 antigen, MTB41 antigen, TbH9 antigen, Ra35 antigen, Ra12 antigen, 38-1 antigen, TbRa3 antigen, 38 kD antigen, DPEP antigen, TbH4 antigen, DPPD antigen, MTB82 antigen, Erd14 antigen, ESAT-6 antigen, MTB85b complex antigen, or an immunogenic fragment thereof.

19. The expression cassette of claim 17, wherein the Mycobacterium polynucleotide sequence encodes for MTB59F antigen, MTB72F antigen, MTB31F antigen, MTB71F antigen, an MTB103F antigen or an immunogenic fragment thereof.

20. The expression cassette of claim 15, wherein the heterologous polynucleotide sequence encodes for MTB8.4 antigen or MTB12 antigen.

21. The expression cassette of claim 14, wherein the Leishmania polynucleotide sequence is located 5′ to the heterologous polynucleotide sequence.

22. The expression cassette of claim 14, wherein the Leishmania polynucleotide sequence is located 3′ to the heterologous polynucleotide sequence.

23. A host cell comprising the expression cassette of claim 1.

24. The host cell of claim 23, wherein the host cell is a eukaryotic cell.

25. A composition comprising an expression cassette comprising a recombinant nucleic acid molecule encoding a fusion polypeptide, the recombinant nucleic acid molecule comprising a heterologous polynucleotide sequence encoding an antigen or an antigenic fragment, and a Leishmania polynucleotide sequence encoding a polypeptide or a fragment thereof, wherein the Leishmania polynucleotide is selected from the group consisting of TSA polynucleotide, LeIF polynucleotide, M15 polynucleotide, and 6H polynucleotide, and wherein the recombinant nucleic acid molecule is operably linked to a eukaryotic promoter.

26. The composition of claim 25, wherein the Leishmania polynucleotide sequence is the TSA polynucleotide.

27. The composition of claim 25, wherein the heterologous polynucleotide sequence encodes a malaria antigen, a cancer antigen, a viral antigen or a bacterial antigen.

28. The composition of claim 27, wherein the heterologous polynucleotide sequence is a Mycobacterium polynucleotide sequence encoding an antigen or antigenic fragment thereof from a Mycobacterium species of the tuberculosis complex.

29. The composition of claim 28, wherein the Mycobacterium polynucleotide sequence encodes for MTB8.4 antigen, MTB9.8 antigen, MTB9.9 antigen, MTB12 antigen, MTB32A antigen, MTB40 antigen, MTB41 antigen, TbH9 antigen, Ra35 antigen, Ra12 antigen, 38-1 antigen, TbRa3 antigen, 38 kD antigen, DPEP antigen, TbH4 antigen, DPPD antigen, MTB82 antigen, Erd14 antigen, ESAT-6 antigen, MTB85b complex antigen, or an immunogenic fragment thereof.

30. The composition of claim 28, wherein the Mycobacterium polynucleotide sequence encodes for MTB59F antigen, MTB72F antigen, MTB31F antigen, MTB71F antigen, MTB103F antigen, or an immunogenic fragment thereof.

31. The composition of claim 26, wherein the heterologous polynucleotide sequence encodes for MTB8.4 antigen or MTB12 antigen.

32. The composition of claim 25, wherein the Mycobacterium polynucleotide sequence is codon optimized for expression in eukaryotic cells.

33. The composition of claim 32, wherein the Leishmania polynucleotide sequence is TSA polynucleotide, LeIF polynucleotide, M15 polynucleotide, or 6H polynucleotide.

34. The composition of claim 33, wherein the Leishmania polynucleotide sequence is the TSA polynucleotide.

35. The composition of claim 32, wherein the heterologous polynucleotide sequence encodes a malaria antigen, a cancer antigen, a viral antigen or a bacterial antigen.

36. The composition of claim 35, wherein the heterologous polynucleotide sequence is a Mycobacterium polynucleotide sequence encoding an antigen or antigenic fragment thereof from a Mycobacterium species of tuberculosis complex.

37. The composition of claim 36, wherein the Mycobacterium polynucleotide sequence encodes for MTB8.4 antigen, MTB9.8 antigen, MTB9.9 antigen, MTB12 antigen, MTB32A antigen, MTB40 antigen, MTB41 antigen, TbH9 antigen, Ra35 antigen, Ra12 antigen, 38-1 antigen, TbRa3 antigen, 38 kD antigen, DPEP antigen, TbH4 antigen, DPPD antigen, MTB82 antigen, Erd14 antigen, ESAT-6 antigen, MTB85b complex antigen, or an immunogenic fragment thereof.

38. The composition of claim 36, wherein the Mycobacterium polynucleotide sequence encodes for MTB59F antigen, MTB72F antigen, MTB31F antigen, MTB71F antigen, an MTB103F antigen, or an immunogenic fragment thereof.

39. The composition of claim 34, wherein the heterologous polynucleotide sequence encodes for MTB8.4 antigen or MTB12 antigen.

40. The composition of claim 25, wherein the composition is effective against infections by multiple microorganisms.

41. The composition of claim 40, wherein the multiple microorganisms are Leishmania and Mycobacterium.

42. A method for eliciting an immune response in a mammal, the method comprising the step of administering to the mammal an immunologically effective amount of an expression cassette comprising a recombinant nucleic acid molecule encoding a fusion polypeptide, the recombinant nucleic acid molecule comprising a heterologous polynucleotide sequence encoding an antigen or an antigenic fragment, and a Leishmania polynucleotide sequence encoding a polypeptide or a fragment thereof, wherein the Leishmania polynucleotide is selected from the group consisting of TSA polynucleotide, LeIF polynucleotide, M15 polynucleotide, and 6H polynucleotide, and wherein the recombinant nucleic acid is operably linked to a eukaryotic promoter.

43. The method of claim 42, wherein the mammal is immunized with BCG.

44. The method of claim 42, wherein the mammal is a human.

45. The method of claim 42, wherein the expression cassette is formulated with an adjuvant.

46. The method of claim 45, wherein the adjuvant comprises QS21 and MPL.

47. The method of claim 45, wherein the adjuvant is selected from the group consisting of AS2, ENHANZYN, MPL, 3D-MPL, IFA, QS21, CWS, TDM, AGP, CPG, Leif, saponin, saponin mimetics, or a combination thereof.

48. The method of claim 42, wherein the Leishmania polynucleotide sequence is the TSA polynucleotide.

49. The method of claim 42, wherein the heterologous polynucleotide sequence encodes a malaria antigen, a cancer antigen, a viral antigen or a bacterial antigen.

50. The method of claim 49, wherein the heterologous polynucleotide sequence is a Mycobacterium polynucleotide encoding an antigen or antigenic fragment thereof from a Mycobacterium species of the tuberculosis complex.

51. The method of claim 50, wherein the Mycobacterium polynucleotide sequence encodes for MTB8.4 antigen, MTB9.8 antigen, MTB9.9 antigen, MTB12 antigen, MTB32A ANTIGEN, MTB40 antigen, MTB41 antigen, TbH9 antigen, Ra35 antigen, Ra12 antigen, 38-1 antigen, TbRa3 antigen, 38 kD antigen, DPEP antigen, TbH4 antigen, DPPD antigen, MTB82 antigen, Erd14 antigen, ESAT-6 antigen, MTB85b complex antigen, or an immunogenic fragment thereof.

52. The method of claim 50, wherein the Mycobacterium polynucleotide sequence encodes for MTB59F antigen, MTB72F antigen, MTB31F antigen, MTB71F antigen, MTB 103F antigen, or an immunogenic fragment thereof.

53. The method of claim 48, wherein the heterologous polynucleotide sequence encodes for MTB8.4 antigen or MTB12 antigen.

54. The method of claim 42, wherein the Mycobacterium polynucleotide sequence is codon optimized for expression in eukaryotic cells.

55. The method of claim 54, wherein the Leishmania polynucleotide sequence is the TSA polynucleotide.

56. The method of claim 42, wherein the heterologous polynucleotide sequence encodes a malaria antigen, a cancer antigen, a viral antigen or a bacterial antigen.

57. The method of claim 56, wherein the heterologous polynucleotide sequence is a Mycobacterium polynucleotide sequence encoding an antigen or antigenic fragment thereof from a Mycobacterium species of the tuberculosis complex.

58. The method of claim 57, wherein the Mycobacterium polynucleotide sequence encodes for MTB8.4 antigen, MTB9.8 antigen, MTB9.9 antigen, MTB12 antigen, MTB32A antigen, MTB40 antigen, MTB41 antigen, TbH9 antigen, Ra35 antigen, Ra12 antigen, 38-1 antigen, TbRa3 antigen, 38 kD antigen, DPEP antigen, TbH4 antigen, DPPD antigen, MTB82 antigen, Erd14 antigen, ESAT-6 antigen, MTB85b complex antigen, or an immunogenic fragment thereof.

59. The method of claim 57, wherein the Mycobacterium polynucleotide sequence encodes for MTB59F antigen, MTB72F antigen, MTB31F antigen, MTB71F antigen, an MTB 103F antigen, or an immunogenic fragment thereof.

60. The method of claim 55, wherein the heterologous polynucleotide sequence encodes for MTB8.4 antigen or MTB12 antigen.

61. The method of claim 42, wherein the expression cassette is effective against infections by multiple microorganisms.

62. The method of claim 61, wherein the microorganisms are Leishmania and Mycobacterium.

63. A recombinant nucleic acid molecule encoding a fusion polypeptide, the recombinant nucleic acid comprising a heterologous polynucleotide sequence encoding an antigen or an antigenic fragment, and a Leishmania polynucleotide encoding a polypeptide or a fragment thereof, wherein the Leishmania polynucleotide is selected from the group consisting of TSA polynucleotide, LeIF polynucleotide, M15 polynucleotide, and 6H polynucleotide.

64. The recombinant nucleic acid of claim 63, wherein the Leishmania polynucleotide sequence is the TSA polynucleotide.

65. The recombinant nucleic acid of claim 63, wherein the heterologous polynucleotide sequence encodes a malaria antigen, a cancer antigen, a viral antigen or a bacterial antigen.

66. The recombinant nucleic acid of claim 65, wherein the heterologous polynucleotide sequence is a Mycobacterium polynucleotide sequence encoding an antigen or antigenic fragment thereof from a Mycobacterium species of tuberculosis complex.

67. The recombinant nucleic acid of claim 66, wherein the Mycobacterium polynucleotide sequence encodes for MTB8.4 antigen, MTB9.8 antigen, MTB9.9 antigen, MTB12 antigen, MTB32A antigen, MTB40 antigen, MTB41 antigen, TbH9 antigen, Ra35 antigen, Ra12 antigen, 38-1 antigen, TbRa3 antigen, 38 kD antigen, DPEP antigen, TbH4 antigen, DPPD antigen, MTB82 antigen, Erd14 antigen, ESAT-6 antigen, MTB85b complex antigen, or an immunogenic fragment thereof.

68. The recombinant nucleic acid of claim 66, wherein the Mycobacterium polynucleotide sequence encodes for MTB59F antigen, MTB72F antigen, MTB31F antigen, MTB71F antigen, MTB103F antigen, or an immunogenic fragment thereof.

69. The recombinant nucleic acid of claim 64, wherein the heterologous polynucleotide sequence encodes for MTB8.4 antigen or MTB12 antigen.

70. The recombinant nucleic acid of claim 63, wherein the Mycobacterium polynucleotide sequence is codon optimized for expression in eukaryotic cells.

71. The recombinant nucleic acid of claim 70, wherein the Leishmania polynucleotide sequence is the TSA polynucleotide.

72. The recombinant nucleic acid of claim 70, wherein the heterologous polynucleotide sequence encodes a malaria antigen, a cancer antigen, a viral antigen or a bacterial antigen.

73. The recombinant nucleic acid of claim 72, wherein the heterologous polynucleotide sequence is a Mycobacterium polynucleotide sequence encoding an antigen or antigenic fragment thereof from a Mycobacterium species of the tuberculosis complex.

74. The recombinant nucleic acid of claim 73, wherein the Mycobacterium polynucleotide sequence encodes for MTB8.4 antigen, MTB9.8 antigen, MTB9.9 antigen, MTB12 antigen, MTB32A antigen, MTB40 antigen, MTB41 antigen, TbH9 antigen, Ra35 antigen, Ra12 antigen, 38-1 antigen, TbRa3 antigen, 38 kD antigen, DPEP antigen, TbH4 antigen, DPPD antigen, MTB82 antigen, Erd14 antigen, ESAT-6 antigen, MTB85b complex antigen, or an immunogenic fragment thereof.

75. The recombinant nucleic acid of claim 73, wherein the Mycobacterium polynucleotide sequence encodes for MTB59F antigen, MTB72F antigen, MTB31F antigen, MTB71F antigen, MTB103F antigen, or an immunogenic fragment thereof.

76. The recombinant nucleic acid of claim 71, wherein the heterologous polynucleotide sequence encodes for MTB8.4 antigen or MTB12 antigen.

77. A fusion polypeptide comprising a heterologous antigen or an antigenic thereof, and a Leishmania polypeptide or a fragment thereof, wherein the Leishmania polypeptide is TSA, LeIF, M15, or 6H.

78. The fusion polypeptide of claim 77, wherein the Leishmania polypeptide is TSA.

79. The fusion polypeptide of claim 77, wherein the heterologous antigen is a malaria antigen, a cancer antigen, a viral antigen, or a bacterial antigen.

80. The fusion polypeptide of claim 79, wherein the heterologous antigen is from a Mycobacterium species of the tuberculosis complex.

81. The fusion polypeptide of claim 80, wherein the Mycobacterium polypeptide is MTB8.4 antigen, MTB9.8 antigen, MTB9.9 antigen, MTB12 antigen, MTB32A antigen, MTB40 antigen, MTB41 antigen, TbH9 antigen, Ra35 antigen, Ra12 antigen, 38-1 antigen, TbRa3 antigen, 38 kD antigen, DPEP antigen, TbH4 antigen, DPPD antigen, MTB82 antigen, Erd14 antigen, ESAT-6 antigen, MTB85b complex antigen, or an immunogenic fragment thereof.

82. The fusion polypeptide of claim 80, wherein the Mycobacterium polypeptide is MTB59F antigen, MTB72F antigen, MTB31F antigen, MTB71F antigen, MTB103F antigen, or an immunogenic fragment thereof.


SEQUENCE LISTING (80 sequences)

SEQ ID NO: 1 - DNA, 1872 bp, Mycobacterium tuberculosis
  MTB32A (Ra35FL)
gactacgttggtgtagaaaaatcctgccgcccggacccttaaggctgggacaatttctgatagctaccccgacacaggaggttacgggatgagcaattcgcgccgccgctcactcaggtggtcatggttgctgagcgtgctggctgccgtcgggctgggcctggccacggcgccggcccaggcggccccgccggccttgtcgcaggaccggttcgccgacttccccgcgctgcccctcgacccgtccgcgatggtcgcccaagtggcgccacaggtggtcaacatcaacaccaaactgggctacaacaacgccgtgggcgccgggaccggcatcgtcatcgatcccaacggtgtcgtgctgaccaacaaccacgtgatcgcgggcgccaccgacatcaatgcgttcagcgtcggctccggccaaacctacggcgtcgatgtggtcgggtatgaccgcacccaggatgtcgcggtgctgcagctgcgcggtgccggtggcctgccgtcggcggcgatcggtggcggcgtcgcggttggtgagcccgtcgtcgcgatgggcaacagcggtgggcagggcggaacgccccgtgcggtgcctggcagggtggtcgcgctcggccaaaccgtgcaggcgtcggattcgctgaccggtgccgaagagacattgaacgggttgatccagttcgatgccgcaatccagcccggtgattcgggcgggcccgtcgtcaacggcctaggacaggtggtcggtatgaacacggccgcgtccgataacttccagctgtcccagggtgggcagggattcgccattccgatcgggcaggcgatggcgatcgcgggccaaatccgatcgggtggggggtcacccaccgttcatatcgggcctaccgccttcctcggcttgggtgttgtcgacaacaacggcaacggcgcacgagtccaacgcgtggtcggaagcgctccggcggcaagtctcggcatctccaccggcgacgtgatcaccgcggtcgacggcgctccgatcaactcggccaccgcgatggcggacgcgcttaacgggcatcatcccggtgacgtcatctcggtgaactggcaaaccaagtcgggcggcacgcgtacagggaacgtgacattggccgagggacccccggcctgatttgtcgcggataccacccgccggccggccaattggattggcgccagccgtgattgccgcgtgagcccccgagttccgtctcccgtgcgcgtggcattgtggaagcaatgaacgaggcagaacacagcgttgagcaccctcccgtgcagggcagttacgtcgaaggcggtgtggtcgagcatccggatgccaaggacttcggcagcgccgccgccctgcccgccgatccgacctggtttaagcacgccgtcttctacgaggtgctggtccgggcgttcttcgacgccagcgcggacggttccgncgatctgcgtggactcatcgatcgcctcgactacctgcagtggcttggcatcgactgcatctgttgccgccgttcctacgactcaccgctgcgcgacggcggttacgacattcgcgacttctacaaggtgctgcccgaattcggcaccgtcgacgatttcgtcgccctggtcgacaccgctcaccggcgaggtatccgcatcatcaccgacctggtgatgaatcacacctcggagtcgcacccctggtttcaggagtcccgccgcgacccagacggaccgtacggtgactattacgtgtggagcgacaccagcgagcgctacaccgacgcccggatcatcttcgtcgacaccgaagagtcgaactggtcattcgatcctgtccgccgacagttnctactggcaccgattctt

SEQ ID NO: 2 - protein, 355 aa, Mycobacterium tuberculosis
  MTB32A (Ra35FL)
MSNSRRRSLRWSWLLSVLAAVGLGLATAPAQAAPPALSQDRFADFPALPLDPSAMVAQVAPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDSGGPVVNGLGQVVGMNTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVNWQTKSGGTRTGNVTLAEGPPA

SEQ ID NO: 3 - DNA, 1002 bp, Artificial Sequence
  Description of Artificial SequenceRa35 mature
catatgcatcaccatcaccatcacgccccgccggccttgtcgcaggaccggttcgccgacttccccgcgctgcccctcgacccgtccgcgatggtcgcccaagtggggccacaggtggtcaacatcaacaccaaactgggctacaacaacgccgtgggcgccgggaccggcatcgtcatcgatcccaacggtgtcgtgctgaccaacaaccacgtgatcgcgggcgccaccgacatcaatgcgttcagcgtcggctccggccaaacctacggcgtcgatgtggtcgggtatgaccgcacccaggatgtcgcggtgctgcagctgcgcggtgccggtggcctgccgtcggcggcgatcggtggcggcgtcgcggttggtgagcccgtcgtcgcgatgggcaacagcggtgggcagggcggaacgccccgtgcggtgcctggcagggtggtcgcgctcggccaaaccgtgcaggcgtcggattcgctgaccggtgccgaagagacattgaacgggttgatccagttcgatgccgcgatccagcccggtgaggcgggcgggcccgtcgtcaacggcctaggacaggtggtcggtatgaacacggccgcgtccgataacttccagctgtcccagggtgggcagggattcgccattccgatcgggcaggcgatggcgatcgcgggccagatccgatcgggtggggggtcacccaccgttcatatcgggcctaccgccttcctcggcttgggtgttgtcgacaacaacggcaacggcgcacgagtccaacgcgtggtcgggagcgctccggcggcaagtctcggcatctccaccggcgacgtgatcaccgcggtcgacggcgctccgatcaactcggccaccgcgatggcggacgcgcttaacgggcatcatcccggtgacgtcatctcggtgacctggcaaaccaagtcgggcggcacgcgtacagggaacgtgacattggccgagggacccccggcctgagaattc

SEQ ID NO: 4 - protein, 330 aa, Artificial Sequence
  Description of Artificial SequenceRa35 mature
MHHHHHHAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDSGGPVVNGLGQVVGMNTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPA

SEQ ID NO: 5 - DNA, 1002 bp, Artificial Sequence
  Description of Artificial SequenceRa35FLMutSA
catatgcatcaccatcaccatcacgccccgccggccttgtcgcaggaccggttcgccgacttccccgcgctgcccctcgacccgtccgcgatggtcgcccaagtggggccacaggtggtcaacatcaacaccaaactgggctacaacaacgccgtgggcgccgggaccggcatcgtcatcgatcccaacggtgtcgtgctgaccaacaaccacgtgatcgcgggcgccaccgacatcaatgcgttcagcgtcggctccggccaaacctacggcgtcgatgtggtcgggtatgaccgcacccaggatgtcgcggtgctgcagctgcgcggtgccggtggcctgccgtcggcggcgatcggtggcggcgtcgcggttggtgagcccgtcgtcgcgatgggcaacagcggtgggcagggcggaacgccccgtgcggtgcctggcagggtggtcgcgctcggccaaaccgtgcaggcgtcggattcgctgaccggtgccgaagagacattgaacgggttgatccagttcgatgccgcgatccagcccggtgatgcgggcgggcccgtcgtcaacggcctaggacaggtggtcggtatgaacacggccgcgtccgataacttccagctgtcccagggtgggcagggattcgccattccgatcgggcaggcgatggcgatcgcgggccagatccgatcgggtggggggtcacccaccgttcatatcgggcctaccgccttcctcggcttgggtgttgtcgacaacaacggcaacggcgcacgagtccaacgcgtggtcgggagcgctccggcggcaagtctcggcatctccaccggcgacgtgatcaccgcggtcgacggcgctccgatcaactcggccaccgcgatggcggacgcgcttaacgggcatcatcccggtgacgtcatctcggtgacctggcaaaccaagtcgggcggcacgcgtacagggaacgtgacattggccgagggacccccggcctgagaattc

SEQ ID NO: 6 - protein, 330 aa, Artificial Sequence
  Description of Artificial SequenceRa35FLMutSA
MHHHHHHAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDAGGPVVNGLGQVVGMNTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPA

SEQ ID NO: 7 - DNA, 585 bp, Artificial Sequence
  Description of Artificial SequenceRa35 (MTB32A N-terminus)
gccccgccggccttgtcgcaggaccggttcgccgacttccccgcgctgcccctcgacccgtccgcgatggtcgcccaagtggggccacaggtggtcaacatcaacaccaaactgggctacaacaacgccgtgggcgccgggaccggcatcgtcatcgatcccaacggtgtcgtgctgaccaacaaccacgtgatcgcgggcgccaccgacatcaatgcgttcagcgtcggctccggccaaacctacggcgtcgatgtggtcgggtatgaccgcacccaggatgtcgcggtgctgcagctgcgcggtgccggtggcctgccgtcggcggcgatcggtggcggcgtcgcggttggtgagcccgtcgtcgcgatgggcaacagcggtgggcagggcggaacgccccgtgcggtgcctggcagggtggtcgcgctcggccaaaccgtgcaggcgtcggattcgctgaccggtgccgaagagacattgaacgggttgatccagttcgatgccgcgatccagcccggtgaggcgggcgggcccgtcgtcaacggcctaggacaggtggtcggtatgaacacggccgcgtcc

SEQ ID NO: 8 - protein, 195 aa, Artificial Sequence
  Description of Artificial SequenceRa35 (MTB32A N-terminus)
APPALSQDRFADFPALPLDPSAMVAQVAPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDSGGPVVNGLGQVVGMNTAAS

SEQ ID NO: 9 - DNA, 447 bp, Artificial Sequence
  Description of Artificial SequenceMTBRa12 (MTB32AC-terminus)
cggtatgaacacggccgcgtccgataacttccagctgtcccagggtgggcagggattcgccattccgatcgggcaggcgatggcgatcgcgggccagatccgatcgggtggggggtcacccaccgttcatatcgggcctaccgccttcctcggcttgggtgttgtcgacaacaacggcaacggcgcacgagtccaacgcgtggtcgggagcgctccggcggcaagtctcggcatctccaccggcgacgtgatcaccgcggtcgacggcgctccgatcaactcggccaccgcgatggcggacgcgcttaacgggcatcatcccggtgacgtcatctcggtgaactggcaaaccaagtcgggcggcacgcgtacagggaacgtgacattggccgagggacccccggcctgatttcgtcgyggataccacccgccggccggccaattgga

SEQ ID NO: 10 - protein, 132 aa, Artificial Sequence
  Description of Artificial SequenceMTBRa12 (MTB32AC-terminus)
TAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVNWQTKSGGTRTGNVTLAEGPPA

SEQ ID NO: 11 - DNA, 851 bp, Mycobacterium tuberculosis
  MTB39 (TbH9)
ctgcagggtggcgtggatgagcgtcaccgcggggcaggccgagctgaccgccgcccaggtccgggttgctgcggcggcctacgagacggcgtatgggctgacggtgcccccgccggtgatcgccgagaaccgtgctgaactgatgattctgatagcgaccaacctcttggggcaaaacaccccggcgatcgcggtcaacgaggccgaatacggcgagatgtgggcccaagacgccgccgcgatgtttggctacgccgcggcgacggcgacggcgacggcgacgttgctgccgttcgaggaggcgccggagatgaccagcgcgggtgggctcctcgagcaggccgccgcggtcgaggaggcctccgacaccgccgcggcgaaccagttgatgaacaatgtgccccaggcgctgaaacagttggcccagcccacgcagggcaccacgccttcttccaagctgggtggcctgtggaagacggtctcgccgcatcggtcgccgatcagcaacatggtgtcgatggccaacaaccacatgtcgatgaccaactcgggtgtgtcgatgaccaacaccttgagctcgatgttgaagggctttgctccggcggcggccgcccaggccgtgcaaaccgcggcgcaaaacggggtccgggcgatgagctcgctgggcagctcgctgggttcttcgggtctgggcggtggggtggccgccaacttgggtcgggcggcctcggtacggtatggtcaccgggatggcggaaaatatgcanagtctggtcggcggaacggtggtccggcgtaaggtttacccccgttttctggatgcggtgaacttcgtcaacggaaacagttac

SEQ ID NO: 12 - protein, 263 aa, Mycobacterium tuberculosis
  MTB39 (TbH9)
VAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALKQLAQPTQGTTPSSKLGGLWKTVSPHRSPISNMVSMANNHMSMTNSGVSMTNTLSSMLKGFAPAAAAQAVQTAAQNGVRAMSSLGSSLGSSGLGGGVAANLGRAASVRYGHRDGGKYAXSGRRNGGPA

SEQ ID NO: 13 - DNA, 3058 bp, Mycobacterium tuberculosis
  MTB39 full length (TbH9FL)
gatcgtacccgtgcgagtgctcgggccgtttgaggatggagtgcacgtgtctttcgtgatggcatacccagagatgttggcggcggcggctgacaccctgcagagcatcggtgctaccactgtggctagcaatgccgctgcggcggccccgacgactggggtggtgccccccgctgccgatgaggtgtcggcgctgactgcggcgcacttcgccgcacatgcggcgatgtatcagtccgtgagcgctcgggctgctgcgattcatgaccagttcgtggccacccttgccagcagcgccagctcgtatgcggccactgaagtcgccaatgcggcggcggccagctaagccaggaacagtcggcacgagaaaccacgagaaatagggacacgtaatggtggatttcggggcgttaccaccggagatcaactccgcgaggatgtacgccggcccgggttcggcctcgctggtggccgcggctcagatgtgggacagcgtggcgagtgacctgttttcggccgcgtcggcgtttcagtcggtggtctggggtctgacggtggggtcgtggataggttcgtcggcgggtctgatggtggcggcggcctcgccgtatgtggcgtggatgagcgtcaccgcggggcaggccgagctgaccgccgcccaggtccgggttgctgcggcggcctacgagacggcgtatgggctgacggtgcccccgccggtgatcgccgagaaccgtgctgaactgatgattctgatagcgaccaacctcttggggcaaaacaccccggcgatcgcggtcaacgaggccgaatacggcgagatgtgggcccaagacgccgccgcgatgtttggctacgccgcggcgacggcgacggcgacggcgacgttgctgccgttcgaggaggcgccggagatgaccagcgcgggtgggctcctcgagcaggccgccgcggtcgaggaggcctccgacaccgccgcggcgaaccagttgatgaacaatgtgccccaggcgctgcaacagctggcccagcccacgcagggcaccacgccttcttccaagctgggtggcctgtggaagacggtctcgccgcatcggtcgccgatcagcaacatggtgtcgatggccaacaaccacatgtcgatgaccaactcgggtgtgtcgatgaccaacaccttgagctcgatgttgaagggctttgctccggcggcggccgcccaggccgtgcaaaccgcggcgcaaaacggggtccgggcgatgagctcgctgggcagctcgctgggttcttcgggtctgggcggtggggtggccgccaacttgggtcgggcggcctcggtcggttcgttgtcggtgccgcaggcctgggccgcggccaaccaggcagtcaccccggcggcgcgggcgctgccgctgaccagcctgaccagcgccgcggaaagagggcccgggcagatgctgggcgggctgccggtggggcagatgggcgccagggccggtggtgggctcagtggtgtgctgcgtgttccgccgcgaccctatgtgatgccgcattctccggcggccggctaggagagggggcgcagactgtcgttatttgaccagtgatcggcggtctcggtgtttccgcggccggctatgacaacagtcaatgtgcatgacaagttacaggtattaggtccaggttcaacaaggagacaggcaacatggcctcacgttttatgacggatccgcacgcgatgcgggacatggcgggccgttttgaggtgcacgcccagacggtggaggacgaggctcgccggatgtgggcgtccgcgcaaaacatttccggtgcgggctggagtggcatggccgaggcgacctcgctagacaccatggcccagatgaatcaggcgtttcgcaacatcgtgaacatgctgcacggggtgcgtgacgggctggttcgcgacgccaacaactacgagcagcaagagcaggcctcccagcagatcctcagcagctaacgtcagccgctgcagcacaatacttttacaagcgaaggagaacaggttcgatgaccatcaactatcaattcggggatgtcgacgctcacggcgccatgatccgcgctcaggccgggttgctggaggccgagcatcaggccatcattcgtgatgtgttgaccgcgagtgacttttggggcggcgccggttcggcggcctgccaggggttcattacccagttgggccgtaacttccaggtgatctacgagcaggccaacgcccacgggcagaaggtgcaggctgccggcaacaacatggcgcaaaccgacagcgccgtcggctccagctgggcctgacaccaggccaaggccagggacgtggtgtacgagtgaagttcctcgcgtgatccttcgggtggcagtctaagtggtcagtgctggggtgttggtggtttgctgcttggcgggttcttcggtgctggtcagtgctgctcgggctcgggtgaggacctcgaggcccaggtagcgccgtccttcgatccattcgtcgtgttgttcggcgaggacggctccgacgaggcggatgatcgaggcgcggtcggggaagatgcccacgacgtcggttcggcgtcgtacctctcggttgaggcgttcctgggggttgttggaccagatttggcgccagatctgcttggggaaggcggtgaacgccagcaggtcggtgcgggcggtgtcgaggtgctcggccaccgcggggagtttgtcggtcagagcgtcgagtacccgatcatattgggcaacaactgattcggcgtcgggctggtcgtagatggagtgcagcagggtgcgcacccacggccaggagggcttcggggtggctgccatcagattggctgcgtagtgggttctgcagcgctgccaggccgctgcgggcagggtggcgccgatcgcggccaccaggccggcgtgggcgtcgctggtgaccagcgcgaccccggacaggccgcgggcgaccaggtcgcggaagaacgccagccagccggccccgtcctcggcggaggtgacctggatgcccaggatc

SEQ ID NO: 14 - protein, 391 aa, Mycobacterium tuberculosis
  MTB39 full length (TbH9FL)
MVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMSMTNSGVSMTNTLSSMLKGFAPAAAAQAVQTAAQNGVRAMSSLGSSLGSSGLGGGVAANLGRAASVGSLSVPQAWAAANQAVTPAARALPLTSLTSAAERGPGQMLGGLPVGQMGARAGGGLSGVLRVPPRPYVMPHSPAAG

SEQ ID NO: 15 - DNA, 2287 bp, Artificial Sequence
  Description of Artificial Sequencetri-fusion protein MTB72F (Ra12-TbH9-Ra35 or MTB32-MTB39 fusion)
tctagaaataattttgtttactttaagaanganatatacatatgcatcaccatcaccatcacacggccgcgtccgataacttccagctgtcccagggtgggcagggattcgccattccgatcgggcaggcgatggcgatcgcgggccagatccgatcgggtggggggtcacccaccgttcatatcgggcctaccgccttcctcggcttgggtgttgtcgacaacaacggcaacggcgcacgagtccaacgcgtggtcgggagcgctccggcggcaagtctcggcatctccaccggcgacgtgatcaccgcggtcgacggcgctccgatcaactcggccaccgcgatggcggacgcgcttaacgggcatcatcccggtgacgtcatctcggtgacctggcaaaccaagtcgggcggcacgcgtacagggaacgtgacattggccgagggacccccggccgaattcatggtggatttcggggcgttaccaccggagatcaactccgcgaggatgtacgccggcccgggttcggcctcgctggtggccgcggctcagatgtgggacagcgtggcgagtgacctgttttcggccgcgtcggcgtttcagtcggtggtctggggtctgacggtggggtcgtggataggttcgtcggcgggtctgatggtggcggcggcctcgccgtatgtggcgtggatgagcgtcaccgcggggcaggccgagctgaccgccgcccaggtccgggttgctgcggcggcctacgagacggcgtatgggctgacggtgcccccgccggtgatcgccgagaaccgtgctgaactgatgattctgatagcgaccaacctcttggggcaaaacaccccggcgatcgcggtcaacgaggccgaatacggcgagatgtgggcccaagacgccgccgcgatgtttggctacgccgcggcgacggcgacggcgacggcgacgttgctgccgttcgaggaggcgccggagatgaccagcgcgggtgggctcctcgagcaggccgccgcggtcgaggaggcctccgacaccgccgcggcgaaccagttgatgaacaatgtgccccaggcgctgcaacagctggcccagcccacgcagggcaccacgccttcttccaagctgggtggcctgtggaagacggtctcgccgcatcggtcgccgatcagcaacatggtgtcgatggccaacaaccacatgtcgatgaccaactcgggtgtgtcgatgaccaacaccttgagctcgatgttgaagggctttgctccggcggcggcccgccaggccgtgcaaaccgcggcgcaaaacggggtccgggcgatgagctcgctgggcagctcgctgggttcttcgggtctgggcggtggggtggccgccaacttgggtcgggcggcctcggtcggttcgttgtcggtgccgcaggcctgggccgcggccaaccaggcagtcaccccggcggcgcgggcgctgccgctgaccagcctgaccagcgccgcggaaagagggcccgggcagatgctgggcgggctgccggtggggcagatgggcgccagggccggtggtgggctcagtggtgtgctgcgtgttccgccgcgaccctatgtgatgccgcattctccggcagccggcgatatcgccccgccggccttgtcgcaggaccggttcgccgacttccccgcgctgcccctcgacccgtccgcgatggtcgcccaagtggggccacaggtggtcaacatcaacaccaaactgggctacaacaacgccgtgggcgccgggaccggcatcgtcatcgatcccaacggtgtcgtgctgaccaacaaccacgtgatcgcgggcgccaccgacatcaatgcgttcagcgtcggctccggccaaacctacggcgtcgatgtggtcgggtatgaccgcacccaggatgtcgcggtgctgcagctgcgcggtgccggtggcctgccgtcggcggcgatcggtggcggcgtcgcggttggtgagcccgtcgtcgcgatgggcaacagcggtgggcagggcggaacgccccgtgcggtgcctggcagggtggtcgcgctcggccaaaccgtgcaggcgtcggattcgctgaccggtgccgaagagacattgaacgggttgatccagttcgatgccgcgatccagcccggtgattcgggcgggcccgtcgtcaacggcctaggacaggtggtcggtatgaacacggccgcgtcctaggatatccatcacactggcggccgctcgagcagatccggntgtaacaaagcccgaaa

SEQ ID NO: 16 - protein, 729 aa, Artificial Sequence
  Description of Artificial Sequencetri-fusion protein MTB72F (Ra12-TbH9-Ra35 or MTB32-MTB39 fusion)
MHHHHHHTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAEFMVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMSMTNSGVSMTNTLSSMLKGFAPAAARQAVQTAAQNGVRAMSSLGSSLGSSGLGGGVAANLGRAASVGSLSVPQAWAAANQAVTPAARALPLTSLTSAAERGPGQMLGGLPVGQMGARAGGGLSGVLRVPPRPYVMPHSPAAGDIAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDSGGPVVNGLGQVVGMNTAAS

SEQ ID NO: 17 - DNA, 2190 bp, Artificial Sequence
  Description of Artificial SequenceMTB72FMutSA (Ra12-TbH9-Ra35MutSA)
atgcatcaccatcaccatcacacggccgcgtccgataacttccagctgtcccagggtgggcagggattcgccattccgatcgggcaggcgatggcgatcgcgggccagatccgatcgggtggggggtcacccaccgttcatatcgggcctaccgccttcctcggcttgggtgttgtcgacaacaacggcaacggcgcacgagtccaacgcgtggtcgggagcgctccggcggcaagtctcggcatctccaccggcgacgtgatcaccgcggtcgacggcgctccgatcaactcggccaccgcgatggcggacgcgcttaacgggcatcatcccggtgacgtcatctcggtgacctggcaaaccaagtcgggcggcacgcgtacagggaacgtgacattggccgagggacccccggccgaattcatggtggatttcggggcgttaccaccggagatcaactccgcgaggatgtacgccggcccgggttcggcctcgctggtggccgcggctcagatgtgggacagcgtggcgagtgacctgttttcggccgcgtcggcgtttcagtcggtggtctggggtctgacggtggggtcgtggataggttcgtcggcgggtctgatggtggcggcggcctcgccgtatgtggcgtggatgagcgtcaccgcggggcaggccgagctgaccgccgcccaggtccgggttgctgcggcggcctacgagacggcgtatgggctgacggtgcccccgccggtgatcgccgagaaccgtgctgaactgatgattctgatagcgaccaacctcttggggcaaaacaccccggcgatcgcggtcaacgaggccgaatacggcgagatgtgggcccaagacgccgccgcgatgtttggctacgccgcggcgacggcgacggcgacggcgacgttgctgccgttcgaggaggcgccggagatgaccagcgcgggtgggctcctcgagcaggccgccgcggtcgaggaggcctccgacaccgccgcggcgaaccagttgatgaacaatgtgccccaggcgctgcaacagctggcccagcccacgcagggcaccacgccttcttccaagctgggtggcctgtggaagacggtctcgccgcatcggtcgccgatcagcaacatggtgtcgatggccaacaaccacatgtcgatgaccaactcgggtgtgtcgatgaccaacaccttgagctcgatgttgaagggctttgctccggcggcggccgcccaggccgtgcaaaccgcggcgcaaaacggggtccgggcgatgagctcgctgggcagctcgctgggttcttcgggtctgggcggtggggtggccgccaacttgggtcgggcggcctcggtcggttcgttgtcggtgccgcaggcctgggccgcggccaaccaggcagtcaccccggcggcgcgggcgctgccgctgaccagcctgaccagcgccgcggaaagagggcccgggcagatgctgggcgggctgccggtggggcagatgggcgccagggccggtggtgggctcagtggtgtgctgcgtgttccgccgcgaccctatgtgatgccgcattctccggcagccggcgatatcgccccgccggccttgtcgcaggaccggttcgccgacttccccgcgctgcccctcgacccgtccgcgatggtcgcccaagtggggccacaggtggtcaacatcaacaccaaactgggctacaacaacgccgtgggcgccgggaccggcatcgtcatcgatcccaacggtgtcgtgctgaccaacaaccacgtgatcgcgggcgccaccgacatcaatgcgttcagcgtcggctccggccaaacctacggcgtcgatgtggtcgggtatgaccgcacccaggatgtcgcggtgctgcagctgcgcggtgccggtggcctgccgtcggcggcgatcggtggcggcgtcgcggttggtgagcccgtcgtcgcgatgggcaacagcggtgggcagggcggaacgccccgtgcggtgcctggcagggtggtcgcgctcggccaaaccgtgcaggcgtcggattcgctgaccggtgccgaagagacattgaacgggttgatccagttcgatgccgcgatccagcccggtgatgcgggcgggcccgtcgtcaacggcctaggacaggtggtcggtatgaacacggccgcgtcctag

SEQ ID NO: 18 - protein, 729 aa, Artificial Sequence
  Description of Artificial SequenceMTB72FMutSA (Ra12-TbH9-Ra35MutSA)
MHHHHHHTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAEFMVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMSMTNSGVSMTNTLSSMLKGFAPAAAAQAVQTAAQNGVRAMSSLGSSLGSSGLGGGVAANLGRAASVGSLSVPQAWAAANQAVTPAARALPLTSLTSAAERGPGQMLGGLPVGQMGARAGGGLSGVLRVPPRPYVMPHSPAAGDIAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDAGGPVVNGLGQVVGMNTAAS

SEQ ID NO: 19 - DNA, 1797 bp, Artificial Sequence
  Description of Artificial Sequencebi-fusion protein TbH9-Ra35 (designated MTB59F)
catatgcatcaccatcaccatcacatggtggatttcggggcgttaccaccggagatcaactccgcgaggatgtacgccggcccgggttcggcctcgctggtggccgcggctcagatgtgggacagcgtggcgagtgacctgttttcggccgcgtcggcgtttcagtcggtggtctggggtctgacggtggggtcgtggataggttcgtcggcgggtctgatggtggcggcggcctcgccgtatgtggcgtggatgagcgtcaccgcggggcaggccgagctgaccgccgcccaggtccgggttgctgcggcggcctacgagacggcgtatgggctgacggtgcccccgccggtgatcgccgagaaccgtgctgaactgatgattctgatagcgaccaacctcttggggcaaaacaccccggcgatcgcggtcaacgaggccgaatacggcgagatgtgggcccaagacgccgccgcgatgtttggctacgccgcggcgacggcgacggcgacggcgacgttgctgccgttcgaggaggcgccggagatgaccagcgcgggtgggctcctcgagcaggccgccgcggtcgaggaggcctccgacaccgccgcggcgaaccagttgatgaacaatgtgccccaggcgctgcaacagctggcccagcccacgcagggcaccacgccttcttccaagctgggtggcctgtggaagacggtctcgccgcatcggtcgccgatcagcaacatggtgtcgatggccaacaaccacatgtcgatgaccaactcgggtgtgtcgatgaccaacaccttgagctcgatgttgaagggctttgctccggcggcggccgcccaggccgtgcaaaccgcggcgcaaaacggggtccgggcgatgagctcgctgggcagctcgctgggttcttcgggtctgggcggtggggtggccgccaacttgggtcgggcggcctcggtcggttcgttgtcggtgccgcaggcctgggccgcggccaaccaggcagtcaccccggcggcgcgggcgctgccgctgaccagcctgaccagcgccgcggaaagagggcccgggcagatgctgggcgggctgccggtggggcagatgggcgccagggccggtggtgggctcagtggtgtgctgcgtgttccgccgcgaccctatgtgatgccgcattctccggcagccggcgatatcgccccgccggccttgtcgcaggaccggttcgccgacttccccgcgctgcccctcgacccgtccgcgatggtcgcccaagtggggccacaggtggtcaacatcaacaccaaactgggctacaacaacgccgtgggcgccgggaccggcatcgtcatcgatcccaacggtgtcgtgctgaccaacaaccacgtgatcgcgggcgccaccgacatcaatgcgttcagcgtcggctccggccaaacctacggcgtcgatgtggtcgggtatgaccgcacccaggatgtcgcggtgctgcagctgcgcggtgccggtggcctgccgtcggcggcgatcggtggcggcgtcgcggttggtgagcccgtcgtcgcgatgggcaacagcggtgggcagggcggaacgccccgtgcggtgcctggcagggtggtcgcgctcggccaaaccgtgcaggcgtcggattcgctgaccggtgccgaagagacattgaacgggttgatccagttcgatgccgcgatccagcccggtgattcgggcgggcccgtcgtcaacggcctaggacaggtggtcggtatgaacacggccgcgtcctaggatatc

SEQ ID NO: 20 - protein, 596 aa, Artificial Sequence
  Description of Artificial Sequencebi-fusion protein TbH9-Ra35 (designated MTB59F)
HMHHHHHHMVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMSMTNSGVSMTNTLSSMLKGFAPAAAAQAVQTAAQNGVRAMSSLGSSLGSSGLGGGVAANLGRAASVGSLSVPQAWAAANQAVTPAARALPLTSLTSAAERGPGQMLGGLPVGQMGARAGGGLSGVLRVPPRPYVMPHSPAAGDIAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDSGGPVVNGLGQVVGMNTAAS

SEQ ID NO: 21 - DNA, 500 bp, Mycobacterium tuberculosis
  MTB8.4 (DPV)
cgtggcaatgtcgttgaccgtcggggccggggtcgcctccgcagatcccgtggacgcggtcattaacaccacctgcaattacgggcaggtagtagctgcgctcaacgcgacggatccgggggctgccgcacagttcaacgcctcaccggtggcgcagtcctatttgcgcaatttcctcgccgcaccgccacctcagcgcgctgccatggccgcgcaattgcaagctgtgccgggggcggcacagtacatcggccttgtcgagtcggttgccggctcctgcaacaactattaagcccatgcgggccccatcccgcgacccggcatcgtcgccggggctaggccagattgccccgctcctcaacgggccgcatcccgcgacccggcatcgtcgccggggctaggccagattgccccgctcctcaacgggccgcatctcgtgccgaattcctgcagcccgggggatccactagttctagagcggccgccaccgcggtggagct

SEQ ID NO: 22 - protein, 96 aa, Mycobacterium tuberculosis
  MTB8.4 (DPV)
VAMSLTVGAGVASADPVDAVINTTCNYGQVVAALNATDPGAAAQFNASPVAQSYLRNFLAAPPPQRAAMAAQLQAVPGAAQYIGLVESVAGSCNNY

SEQ ID NO: 23 - DNA, 585 bp, Mycobacterium tuberculosis
  MTB9.8 (MSL)
tggattccgatagcggtttcggcccctcgacgggcgaccacggcgcgcaggcctccgaacggggggccgggacgctgggattcgccgggaccgcaaccaaagaacgccgggtccgggcggtcgggctgaccgcactggccggtgatgagttcggcaacggcccccggatgccgatggtgccggggacctgggagcagggcagcaacgagcccgaggcgcccgacggatcggggagagggggaggcgacggcttaccgcacgacagcaagtaaccgaattccgaatcacgtggacccgtacgggtcgaaaggagagatgttatgagccttttggatgctcatatcccacagttggtggcctcccagtcggcgtttgccgccaaggcggggctgatgcggcacacgatcggtcaggccgagcaggcggcgatgtcggctcaggcgtttcaccagggggagtcgtcggcggcgtttcaggccgcccatgcccggtttgtggcggcggccgccaaagtcaacaccttgttggatgtcgcgcaggcgaatctgggtgaggccgccggtacctatgtggccgccgatgctg

SEQ ID NO: 24 - protein, 97 aa, Mycobacterium tuberculosis
  MTB9.8 (MSL)
MSLLDAHIPQLVASQSAFAAKAGLMRHTIGQAEQAAMSAQAFHQGESSAAFQAAHARFVAAAAKVNTLLDVAQANLGEAAGTYVAADAAAASTYTGF

SEQ ID NO: 25 - DNA, 1742 bp, Mycobacterium tuberculosis
  MTB9.9A (MTI, also known as variant MTI-A)
ccgctctctttcaacgtcataagttcggtgggccagtcggccgcgcgtgcatatggcaccaataacgcgtgtcccatggatacccggaccgcacgacggtagagcggatcagcgcagccggtgccgaacactaccgcgtccacgctcagccctgccgcgttgcggaagatcgagcccaggttctcatggtcgttaacgccttccaacactgcgacggtgcgcgccccggcgaccacctgagcaacgctcggctccggcacccggcgcgcggctgccaacaccccacgattgagatggaagccgatcacccgtgccatgacatcagccgacgctcgatagtacggcgcgccgacaccggccagatcatccttgagctcggccagccggcggtcggtgccgaacagcgccagcggcgtgaaccgtgaggccagcatgcgctgcaccaccagcacaccctcggcgatcaccaacgccttgccggtcggcagatcgggacnacngtcgatgctgttcaggtcacggaaatcgtcgagccgtgggtcgtcgggatcgcagacgtcctgaacatcgaggccgtcggggtgctgggcacaacggccttcggtcacgggctttcgtcgaccagagccagcatcagatcggcggcgctgcgcaggatgtcacgctcgctgcggttcagcgtcgcgagccgctcagccagccactcttgcagagagccgttgctgggattaattgggagaggaagacagcatgtcgttcgtgaccacacagccggaagccctggcagctgcggcggcgaacctacagggtattggcacgacaatgaacgcccagaacgcggccgcggctgctccaaccaccggagtagtgcccgcagccgccgatgaagtatcagcgctgaccgcggctcagtttgctgcgcacgcgcagatgtaccaaacggtcagcgcccaggccgcggccattcacgaaatgttcgtgaacacgctggtggccagttctggctcatacgcggccaccgaggcggccaacgcagccgctgccggctgaacgggctcgcacgaacctgctgaaggagagggggaacatccggagttctcgggtcaggggttgcgccagcgcccagccgattcagntatcggcgtccataacagcagacgatctaggcattcagtactaaggagacaggcaacatggcctcacgttttatgacggatccgcatgcgatgcgggacatggcgggccgttttgaggtgcacgcccagacggtggaggacgaggctcgccggatgtgggcgtccgcgcaaaacatttccggtgcgggctggagtggcatggccgaggcgacctcgctagacaccatgacctagatgaatcaggcgtttcgcaacatcgtgaacatgctgcacggggtgcgtgacgggctggttcgcgacgccaacaantacgaacagcaagagcaggcctcccagcagatcctgagcagntagcgccgaaagccacagctgngtacgntttctcacattaggagaacaccaatatgacgattaattaccagttcggggacgtcgacgctcatggcgccatgatccgcgctcaggcggcgtcgcttgaggcggagcatcaggccatcgttcgtgatgtgttggccgcgggtgacttttggggcggcgccggttcggtggcttgccaggagttcattacccagttgggccgtaacttccaggtgatctacgagcagg

SEQ ID NO: 26 - DNA, 2836 bp, Mycobacterium tuberculosis
  MTB9.9A (MTI, also known as variant MTI-A)
gttgattccgttcgcggcgccgccgaagaccaccaactccgctggggtggtcgcacaggcggttgcgtcggtcagctggccgaatcccaatgattggtggctcngtgcggttgctgggctcgattacccccacggaaaggacgacgatcgttcgtttgctcggtcagtcgtacttggcgacgggcatggcgcggtttcttacctcgatcgcacagcagctgaccttcggcccagggggcacaacggctggctccggcggagcctggtacccaacgccacaattcgccggcctgggtgcaggcccggcggtgtcggcgagtttggcgcgggcggagccggtcgggaggttgtcggtgccgccaagttgggccgtcgcggctccggccttcgcggagaagcctgaggcgggcacgccgatgtccgtcatcggcgaagcgtccagctgcggtcagggaggcctgcttcgaggcataccgctggcgagagcggggcggcgtacaggcgccttcgctcaccgatacgggttccgccacagcgtgattacccggtctccgtcggcgggatagctttcgatccggtctgcgcggccgccggaaatgctgcagatagcgatcgaccgcgccggtcggtaaacgccgcacacggcactatcaatgcgcacggcgggcgttgatgccaaattgaccgtcccgacggggctttatctgcggcaagatttcatccccagcccggtcggtgggccgataaatacgctggtcagcgcgactcttccggctgaattcgatgctctgggcgcccgctcgacgccgagtatctcgagtgggccgcaaacccggtcaaacgctgttactgtggcgttaccacaggtgaatttgcggtgccaactggtgaacacttgcgaacgggtggcatcgaaatcaacttgttgcgttgcagtgatctactctcttgcagagagccgttgctgggattaattgggagaggaagacagcatgtcgttcgtgaccacacagccggaagccctggcagctgcggcggcgaacctacagggtattggcacgacaatgaacgcccagaacgcggccgcggctgctccaaccaccggagtagtgcccgcagccgccgatgaagtatcagcgctgaccgcggctcagtttgctgcgcacgcgcagatgtaccaaacggtcagcgcccaggccgcggccattcacgaaatgttcgtgaacacgctggtggccagttctggctcatacgcggccaccgaggcggccaacgcagccgctgccggctgaacgggctcgcacgaacctgctgaaggagagggggaacatccggagttctcgggtcaggggttgcgccagcgcccagccgattcagctatcggcgtccataacagcagacgatctaggcattcagtactaaggagacaggcaacatggcctcacgttttatgacggatccgcatgcgatgcgggacatggcgggccgttttgaggtgcacgcccagacggtggaggacgaggctcgccggatgtgggcgtccgcgcaaaacatttccggtgcgggctggagtggcatggccgaggcgacctcgctagacaccatgacctagatgaatcaggcgtttcgcaacatcgtgaacatgctgcacggggtgcgtgacgggctggttcgcgacgccaacaactacgaacagcaagagcaggcctcccagcagatcctgagcagctagcgccgaaagccacagctgcgtacgctttctcacattaggagaacaccaatatgacgattaattaccagttcggggacgtcgacgctcatggcgccatgatccgcgctcaggcggcgtcgcttgaggcggagcatcaggccatcgttcgtgatgtgttggccgcgggtgacttttggggcggcgccggttcggtggcttgccaggagttcattacccagttgggccgtaacttccaggtgatctacgagcaggccaacgcccacgggcagaaggtgcaggctgccggcaacaacatggcgcaaaccgacagcgccgtcggctccagctgggcctaaaactgaacttcagtcgcggcagcacaccaaccagccggtgtgctgctgtgtcctgcagttaactagcactcgaccgctgaggtagcgatggatcaacagagtacccgcaccgacatcaccgtcaacgtcgacggcttctggatgcttcaggcgctactggatatccgccacgttgcgcctgagttacgttgccggccgtacgtctccaccgattccaatgactggctaaacgagcacccggggatggcggtcatgcgcgagcagggcattgtcgtcaacgacgcggtcaacgaacaggtcgctgcccggatgaaggtgcttgccgcacctgatcttgaagtcgtcgccctgctgtcacgcggcaagttgctgtacggggtcatagacgacgagaaccagccgccgggttcgcgtgacatccctgacaatgagttccgggtggtgttggcccggcgaggccagcactgggtgtcggcggtacgggttggcaatgacatcaccgtcgatgacgtgacggtctcggatagcgcctcgatcgccgcactggtaatggacggtctggagtcgattcaccacgccgacccagccgcgatcaacgcggtcaacgtgccaatggaggagatctcgtgccgaattcggcacgaggcacgaggcggtgtcggtgacgacgggatcgatcacgatcatcgaccggccgggatccttggcgatctcgttgagcacgacccgggcccgcgggaagctctgcgacatccatgggttcttcccg

SEQ ID NO: 27 - protein, 94 aa, Mycobacterium tuberculosis
  MTB9.9A (MTI, also known as variant MTI-A)
MTINYQFGDVDAHGAMIRALAGLLEAEHQAIISDVLTASDFWGGAGSAACQGFITQLGRNFQVIYEQANAHGQKVQAAGNNMAQTDSAVGSSWA

SEQ ID NO: 28 - DNA, 1200 bp, Mycobacterium tuberculosis
  MTB40 (HTCC#1)
caggcatgagcagagcgttcatcatcgatccaacgatcagtgccattgacggcttgtacgaccttctggggattggaatacccaaccaagggggtatcctttactcctcactagagtacttcgaaaaagccctggaggagctggcagcagcgtttccgggtgatggctggttaggttcggccgcggacaaatacgccggcaaaaaccgcaaccacgtgaattttttccaggaactggcagacctcgatcgtcagctcatcagcctgatccacgaccaggccaacgcggtccagacgacccgcgacatcctggagggcgccaagaaaggtctcgagttcgtgcgcccggtggctgtggacctgacctacatcccggtcgtcgggcacgccctatcggccgccttccaggcgccgttttgcgcgggcgcgatggccgtagtgggcggcgcgcttgcctacttggtcgtgaaaacgctgatcaacgcgactcaactcctcaaattgcttgccaaattggcggagttggtcgcggccgccattgcggacatcatttcggatgtggcggacatcatcaagggcaccctcggagaagtgtgggagttcatcacaaacgcgctcaacggcctgaaagagctttgggacaagctcacggggtgggtgaccggactgttctctcgagggtggtcgaacctggagtccttctttgcgggcgtccccggcttgaccggcgcgaccagcggcttgtcgcaagtgactggcttgttcggtgcggccggtctgtccgcatcgtcgggcttggctcacgcggatagcctggcgagctcagccagcttgcccgccctggccggcattgggggcgggtccggttttgggggcttgccgagcctggctcaggtccatgccgcctcaactcggcaggcgctacggccccgagctgatggcccggtcggcgccgctgccgagcaggtcggcgggcagtcgcagctggtctccgcgcagggttcccaaggtatgggcggacccgtaggcatgggcggcatgcacccctcttcgggggcgtcgaaagggacgacgacgaagaagtactcggaaggcgcggcggcgggcactgaagacgccgagcgcgcgccagtcgaagctgacgcgggcggtgggcaaaaggtgctggtacgaaacgtcgtctaacggcatggcgagccaa

SEQ ID NO: 29 - protein, 392 aa, Mycobacterium tuberculosis
  MTB40 (HTCC#1)
MSRAFIIDPTISAIDGLYDLLGIGIPNQGGILYSSLEYFEKALEELAAAFPGDGWLGSAADKYAGKNRNHVNFFQELADLDRQLISLIHDQANAVQTTRDILEGAKKGLEFVRPVAVDLTYIPVVGHALSAAFQAPFCAGAMAVVGGALAYLVVKTLINATQLLKLLAKLAELVAAAIADIISDVADIIKGTLGEVWEFITNALNGLKELWDKLTGWVTGLFSRGWSNLESFFAGVPGLTGATSGLSQVTGLFGAAGLSASSGLAHADSLASSASLPALAGIGGGSGFGGLPSLAQVHAASTRQALRPRADGPVGAAAEQVGGQSQLVSAQGSQGMGGPVGMGGMHPSSGASKGTTTKKYSEGAAAGTEDAERAPVEADAGGGQKVLVRNVV

SEQ ID NO: 30 - DNA, 1441 bp, Mycobacterium tuberculosis
  MTB41 (MTCC#2)
gaggttgctggcaatggatttcgggcttttacctccggaagtgaattcaagccgaatgtattccggtccggggccggagtcgatgctagccgccgcggccgcctgggacggtgtggccgcggagttgacttccgccgcggtctcgtatggatcggtggtgtcgacgctgatcgttgagccgtggatggggccggcggcggccgcgatggcggccgcggcaacgccgtatgtggggtggctggccgccacggcggcgctggcgaaggagacggccacacaggcgagggcagcggcggaagcgtttgggacggcgttcgcgatgacggtgccaccatccctcgtcgcggccaaccgcagccggttgatgtcgctggtcgcggcgaacattctggggcaaaacagtgcggcgatcgcggctacccaggccgagtatgccgaaatgtgggcccaagacgctgccgtgatgtacagctatgagggggcatctgcggccgcgtcggcgttgccgccgttcactccacccgtgcaaggcaccggcccggccgggcccgcggccgcagccgcggcgacccaagccgccggtgcgggcgccgttgcggatgcacaggcgacactggcccagctgcccccggggatcctgagcgacattctgtccgcattggccgccaacgctgatccgctgacatcgggactgttggggatcgcgtcgaccctcaacccgcaagtcggatccgctcagccgatagtgatccccaccccgataggggaattggacgtgatcgcgctctacattgcatccatcgcgaccggcagcattgcgctcgcgatcacgaacacggccagaccctggcacatcggcctatacgggaacgccggcgggctgggaccgacgcagggccatccactgagttcggcgaccgacgagccggagccgcactggggccccttcgggggcgcggcgccggtgtccgcgggcgtcggccacgcagcattagtcggagcgttgtcggtgccgcacagctggaccacggccgccccggagatccagctcgccgttcaggcaacacccaccttcagctccagcgccggcgccgacccgacggccctaaacgggatgccggcaggcctgctcagcgggatggctttggcgagcctggccgcacgcggcacgacgggcggtggcggcacccgtagcggcaccagcactgacggccaagaggacggccgcaaacccccggtagttgtgattagagagcagccgccgcccggaaaccccccgcggtaaaagtccggcaaccgttcgtcgccgcgcggaaaatgcctggtgagcgtggctatccgacgggccgttcacaccgcttgtagtagcgtacggctatggacgacggtgtctggattctcggcggctatcagagcgattttgctcgcaacctcagcaaag

SEQ ID NO: 31 - protein, 423 aa, Mycobacterium tuberculosis
  MTB41 (MTCC#2)
MDFGLLPPEVNSSRMYSGPGPESMLAAAAAWDGVAAELTSAAVSYGSVVSTLIVEPWMGPAAAAMAAAATPYVGWLAATAALAKETATQARAAAEAFGTAFAMTVPPSLVAANRSRLMSLVAANILGQNSAAIAATQAEYAEMWAQDAAVMYSYEGASAAASALPPFTPPVQGTGPAGPAAAAAATQAAGAGAVADAQATLAQLPPGILSDILSALAANADPLTSGLLGIASTLNPQVGSAQPIVIPTPIGELDVIALYIASIATGSIALAITNTARPWHIGLYGNAGGLGPTQGHPLSSATDEPEPHWGPFGGAAPVSAGVGHAALVGALSVPHSWTTAAPEIQLAVQATPTFSSSAGADPTALNGMPAGLLSGMALASLAARGTTGGGGTRSGTSTDGQEDGRKPPVVVIREQPPPGNPPR

SEQ ID NO: 32 - DNA, 154 bp, Mycobacterium tuberculosis
  ESAT-6
atgacagagcagcagtggaatttcgcgggtatcgaggccgcggcaagcgcaatccagggaaatgtcacgtccattcattccctccttgacgaggggaagcagtccctgaccaagctcgcagcggcctggggcggtagcggttcggaagcgtacc

SEQ ID NO: 33 - protein, 51 aa, Mycobacterium tuberculosis
  ESAT-6
MTEQQWNFAGIEAAASAIQGNVTSIHSLLDEGKQSLTKLAAAWGGSGSEAY

SEQ ID NO: 34 - DNA, 327 bp, Mycobacterium tuberculosis
  Tb38-1 or 38-1 (MTb11)
cggcacgagagaccgatgccgctaccctcgcgcaggaggcaggtaatttcgagcggatctccggcgacctgaaaacccagatcgaccaggtggagtcgacggcaggttcgttgcagggccagtggcgcggcgcggcggggacggccgcccaggccgcggtggtgcgcttccaagaagcagccaataagcagaagcaggaactcgacgagatctcgacgaatattcgtcaggccggcgtccaatactcgagggccgacgaggagcagcagcaggcgctgtcctcgcaaatgggcttctgacccgctaatacgaaaagaaacggagcaa

SEQ ID NO: 35 - protein, 95 aa, Mycobacterium tuberculosis
  Tb38-1 or 38-1 (MTb11)
TDAATLAQEAGNFERISGDLKTQIDQVESTAGSLQGQWRGAAGTAAQAAVVRFQEAANKQKQELDEISTNIRQAGVQYSRADEEQQQALSSQMGF

SEQ ID NO: 36 - DNA, 542 bp, Mycobacterium tuberculosis
  TbRa3
gaattcggcacgagaggtgatcgacatcatcgggaccagccccacatcctgggaacaggcggcggcggaggcggtccagcgggcgcgggatagcgtcgatgacatccgcgtcgctcgggtcattgagcaggacatggccgtggacagcgccggcaagatcacctaccgcatcaagctcgaagtgtcgttcaagatgaggccggcgcaaccgcgctagcacgggccggcgagcaagacgcaaaatcgcacggtttgcggttgattcgtgcgattttgtgtctgctcgccgaggcctaccaggcgcggcccaggtccgcgtgctgccgtatccaggcgtgcatcgcgattccggcggccacgccggagttaatgcttcgcgtcgacccgaactgggcgatccgccggngagctgatcgatgaccgtggccagcccgtcgatgcccgagttgcccgaggaaacgtgctgccaggccggtaggaagcgtccgtaggcggcggtgctgaccggctctgcctgcgccctcagtgcggccagcgagcgg

SEQ ID NO: 37 - protein, 66 aa, Mycobacterium tuberculosis
  TbRa3
VIDIIGTSPTSWEQAAAEAVQRARDSVDDIRVARVIEQDMAVDSAGKITYRIKLEVSFKMRPAQPR

SEQ ID NO: 38 - DNA, 1993 bp, Mycobacterium tuberculosis
  38 kD
tgttcttcgacggcaggctggtggaggaagggcccaccgaacagctgttctcctcgccgaagcatgcggaaaccgcccgatacgtcgccggactgtcgggggacgtcaaggacgccaagcgcggaaattgaagagcacagaaaggtatggcgtgaaaattcgtttgcatacgctgttggccgtgttgaccgctgcgccgctgctgctagcagcggcgggctgtggctcgaaaccaccgagcggttcgcctgaaacgggcgccggcgccggtactgtcgcgactacccccgcgtcgtcgccggtgacgttggcggagaccggtagcacgctgctctacccgctgttcaacctgtggggtccggcctttcacgagaggtatccgaacgtcacgatcaccgctcagggcaccggttctggtgccgggatcgcgcaggccgccgccgggacggtcaacattggggcctccgacgcctatctgtcggaaggtgatatggccgcgcacaaggggctgatgaacatcgcgctagccatctccgctcagcaggtcaactacaacctgcccggagtgagcgagcacctcaagctgaacggaaaagtcctggcggccatgtaccagggcaccatcaaaacctgggacgacccgcagatcgctgcgctcaaccccggcgtgaacctgcccggcaccgcggtagttccgctgcaccgctccgacgggtccggtgacaccttcttgttcacccagtacctgtccaagcaagatcccgagggctggggcaagtcgcccggcttcggcaccaccgtcgacttcccggcggtgccgggtgcgctgggtgagaacggcaacggcggcatggtgaccggttgcgccgagacaccgggctgcgtggcctatatcggcatcagcttcctcgaccaggccagtcaacggggactcggcgaggcccaactaggcaatagctctggcaatttcttgttgcccgacgcgcaaagcattcaggccgcggcggctggcttcgcatcgaaaaccccggcgaaccaggcgatttcgatgatcgacgggcccgccccggacggctacccgatcatcaactacgagtacgccatcgtcaacaaccggcaaaaggacgccgccaccgcgcagaccttgcaggcatttctgcactgggcgatcaccgacggcaacaaggcctcgttcctcgaccaggttcatttccagccgctgccgcccgcggtggtgaagttgtctgacgcgttgatcgcgacgatttccagctagcctcgttgaccaccacgcgacagcaacctccgtcgggccatcgggctgctttgcggagcatgctggcccgtgccggtgaagtcggccgcgctggcccggccatccggtggttgggtgggataggtgcggtgatcccgctgcttgcgctggtcttggtgctggtggtgctggtcatcgaggcgatgggtgcgatcaggctcaacgggttgcatttcttcaccgccaccgaatggaatccaggcaacacctacggcgaaaccgttgtcaccgacgcgtcgcccatccggtcggcgcctactacggggcgttgccgctgatcgtcgggacgctggcgacctcggcaatcgccctgatcatcgcggtgccggtctctgtaggagcggcgctggtgatcgtggaacggctgccgaaacggttggccgaggctgtgggaatagtcctggaattgctcgccggaatccccagcgtggtcgtcggtttgtggggggcaatgacgttcgggccgttcatcgctcatcacatcgctccggtgatcgctcacaacgctcccgatgtgccggtgctgaactacttgcgcggcgacccgggcaacggggagggcatgttggtgtccggtctggtgttggcggtgatggtcgttcccattatcgccaccaccactcatgacctgttccggcaggtgccggtgttgccccgggagggcgcgatcgggaattc

SEQ ID NO: 39 - protein, 374 aa, Mycobacterium tuberculosis
  38 kD
MKIRLHTLLAVLTAAPLLLAAAGCGSKPPSGSPETGAGAGTVATTPASSPVTLAETGSTLLYPLFNLWGPAFHERYPNVTITAQGTGSGAGIAQAAAGTVNIGASDAYLSEGDMAAHKGLMNIALAISAQQVNYNLPGVSEHLKLNGKVLAAMYQGTIKTWDDPQIAALNPGVNLPGTAVVPLHRSDGSGDTFLFTQYLSKQDPEGWGKSPGFGTTVDFPAVPGALGENGNGGMVTGCAETPGCVAYIGISFLDQASQRGLGEAQLGNSSGNFLLPDAQSIQAAAAGFASKTPANQAISMIDGPAPDGYPIINYEYAIVNNRQKDAATAQTLQAFLHWAITDGNKASFLDQVHFQPLPPAVVKLSDALIATISS

SEQ ID NO: 40 - DNA, 999 bp, Artificial Sequence
  Description of Artificial SequenceDPEP
atgcatcaccatcaccatcacatgcatcaggtggaccccaacttgacacgtcgcaagggacgattggcggcactggctatcgcggcgatggccagcgccagcctggtgaccgttgcggtgcccgcgaccgccaacgccgatccggagccagcgcccccggtacccacaacggccgcctcgccgccgtcgaccgctgcagcgccacccgcaccggcgacacctgttgcccccccaccaccggccgccgccaacacgccgaatgcccagccgggcgatcccaacgcagcacctccgccggccgacccgaacgcaccgccgccacctgtcattgccccaaacgcaccccaacctgtccggatcgacaacccggttggaggattcagcttcgcgctgcctgctggctgggtggagtctgacgccgcccacttcgactacggttcagcactcctcagcaaaaccaccggggacccgccatttcccggacagccgccgccggtggccaatgacacccgtatcgtgctcggccggctagaccaaaagctttacgccagcgccgaagccaccgactccaaggccgcggcccggttgggctcggacatgggtgagttctatatgccctacccgggcacccggatcaaccaggaaaccgtctcgctcgacgccaacggggtgtctggaagcgcgtcgtattacgaagtcaagttcagcgatccgagtaagccgaacggccagatctggacgggcgtaatcggctcgcccgcggcgaacgcaccggacgccgggccccctcagcgctggtttgtggtatggctcgggaccgccaacaacccggtggacaagggcgcggccaaggcgctggccgaatcgatccggcctttggtcgccccgccgccggcgccggcaccggctcctgcagagcccgctccggcgccggcgccggccggggaagtcgctcctaccccgacgacaccgacaccgcagcggaccttaccggcctga

SEQ ID NO: 41 - protein, 332 aa, Artificial Sequence
  Description of Artificial SequenceDPEP
MHHHHHHMHQVDPNLTRRKGRLAALAIAAMASASLVTVAVPATANADPEPAPPVPTTAASPPSTAAAPPAPATPVAPPPPAAANTPNAQPGDPNAAPPPADPNAPPPPVIAPNAPQPVRIDNPVGGFSFALPAGWVESDAAHFDYGSALLSKTTGDPPFPGQPPPVANDTRIVLGRLDQKLYASAEATDSKAAARLGSDMGEFYMPYPGTRINQETVSLDANGVSGSASYYEVKFSDPSKPNGQIWTGVIGSPAANAPDAGPPQRWFVVWLGTANNPVDKGAAKALAESIRPLVAPPPAPAPAPAEPAPAPAPAGEVAPTPTTPTPQRTLPA

SEQ ID NO: 42 - DNA, 702 bp, Mycobacterium tuberculosis
  TbH4
cggcacgaggatcggtaccccgcggcatcggcagctgccgattcgccgggtttccccacccgaggaaagccgctaccagatggcgctgccgaagtagggcgatccgttcgcgatgccggcatgaacgggcggcatcaaattagtgcaggaacctttcagtttagcgacgataatggctatagcactaaggaggatgatccgatatgacgcagtcgcagaccgtgacggtggatcagcaagagattttgaacagggccaacgaggtggaggccccgatggcggacccaccgactgatgtccccatcacaccgtgcgaactcacggnggntaaaaacgccgcccaacagntggtnttgtccgccgacaacatgcgggaatacctggcggccggtgccaaagagcggcagcgtctggcgacctcgctgcgcaacgcggccaaggngtatggcgaggttgatgaggaggctgcgaccgcgctggacaacgacggcgaaggaactgtgcaggcagaatcggccggggccgtcggaggggacagttcggccgaactaaccgatacgccgagggtggccacggccggtgaacccaacttcatggatctcaaagaagcggcaaggaagctcgaaacgggcgaccaaggcgcatcgctcgcgcactgnggggatgggtggaacacttncaccctgacgctgcaaggcgacg

SEQ ID NO: 43 - protein, 286 aa, Mycobacterium tuberculosis
  TbH4
GDSFWAAADQMARGFVLGATAGRTTLTGEGLQHADGHSLLLDATNPAVVAYDPAFAYEIGYIXESGLARMCGENPENIFFYITVYNEPYVQPPEPENFDPEGVLGGIYRYHAATEQRTNKXQILASGVAMPAALRAAQMLAAEWDVAADVWSVTSWGELNRDGVVIETEKLRHPDRPAGVPYVTRALENARGPVIAVSDWMRAVPEQIRPWVPGTYLTLGTDGFGFSDTRPAGRRYFNTDAESQVGRGFGRGWPGRRVNIDPFGAGRGPPAQLPGFDEGGGLRPXK

SEQ ID NO: 44 - DNA, 339 bp, Mycobacterium tuberculosis
  DPPD
atgaagttgaagtttgctcgcctgagtactgcgatactgggttgtgcagcggcgcttgtgtttcctgcctcggttgccagcgcagatccacctgacccgcatcagccggacatgacgaaaggctattgcccgggtggccgatggggttttggcgacttggccgtgtgcgacggcgagaagtaccccgacggctcgttttggcaccagtggatgcaaacgtggtttaccggcccacagttttacttcgattgtgtcagcggcggtgagcccctccccggcccgccgccaccgggtggttgcggtggggcaattccgtccgagcagcccaacgctccctga

SEQ ID NO: 45 - protein, 112 aa, Mycobacterium tuberculosis
  DPPD
MKLKFARLSTAILGCAAALVFPASVASADPPDPHQPDMTKGYCPGGRWGFGDLAVCDGEKYPDGSFWHQWMQTWFTGPQFYFDCVSGGEPLPGPPPPGGCGGAIPSEQPNAP

SEQ ID NO: 46 - DNA, 921 bp, Artificial Sequence
  Description of Artificial Sequencetri-fusion protein DPV-MTI-MSL (designated (MTb31F)
catatgcatcaccatcaccatcacgatcccgtggacgcggtcattaacaccacctgcaattacgggcaggtagtagctgcgctcaacgcgacggatccgggggctgccgcacagttcaacgcctcaccggtggcgcagtcctatttgcgcaatttcctcgccgcaccgccacctcagcgcgctgccatggccgcgcaattgcaagctgtgccgggggcggcacagtacatcggccttgtcgagtcggttgccggctcctgcaacaactatgagctcatgacgattaattaccagttcggggacgtcgacgctcatggcgccatgatccgcgctcaggcggcgtcgcttgaggcggagcatcaggccatcgttcgtgatgtgttggccgcgggtgacttttggggcggcgccggttcggtggcttgccaggagttcattacccagttgggccgtaacttccaggtgatctacgagcaggccaacgcccacgggcagaaggtgcaggctgccggcaacaacatggcgcaaaccgacagcgccgtcggctccagctgggccactagtatgagccttttggatgctcatatcccacagttggtggcctcccagtcggcgtttgccgccaaggcggggctgatgcggcacacgatcggtcaggccgagcaggcggcgatgtcggctcaggcgtttcaccagggggagtcgtcggcggcgtttcaggccgcccatgcccggtttgtggcggcggccgccaaagtcaacaccttgttggatgtcgcgcaggcgaatctgggtgaggccgccggtacctatgtggccgccgatgctgcggccgcgtcgacctataccgggttcgatatccatcacactggcggccgctcgagcagatccggctgctaacaaagcccgaaaggaagctga

SEQ ID NO: 47 - protein, 299 aa, Artificial Sequence
  Description of Artificial Sequencetri-fusion protein DPV-MTI-MSL (designated (MTb31F)
HMHHHHHHDPVDAVINTTCNYGQVVAALNATDPGAAAQFNASPVAQSYLRNFLAAPPPQRAAMAAQLQAVPGAAQYIGLVESVAGSCNNYELMTINYQFGDVDAHGAMIRAQAASLEAEHQAIVRDVLAAGDFWGGAGSVACQEFITQLGRNFQVIYEQANAHGQKVQAAGNNMAQTDSAVGSSWATSMSLLDAHIPQLVASQSAFAAKAGLMRHTIGQAEQAAMSAQAFHQGESSAAFQAAHARFVAAAAKVNTLLDVAQANLGEAAGTYVAADAAAASTYTGFDIHHTGGRSSRSGC

SEQ ID NO: 48 - DNA, 2168 bp, Artificial Sequence
  Description of Artificial Sequencetetra-fusion protein DPV-MTI-MSL-MTCC#2 (designated (MTb71F)
catatgcatcaccatcaccatcacgatcccgtggacgcggtcattaacaccacctgcaattacgggcaggtagtagctgcgctcaacgcgacggatccgggggctgccgcacagttcaacgcctcaccggtggcgcagtcctatttgcgcaatttcctcgccgcaccgccacctcagcgcgctgccatggccgcgcaattgcaagctgtgccgggggcggcacagtacatcggccttgtcgagtcggttgccggctcctgcaacaactatgagctcatgacgattaattaccagttcggggacgtcgacgctcatggcgccatgatccgcgctcaggcggcgtcgcttgaggcggagcatcaggccatcgttcgtgatgtgttggccgcgggtgacttttggggcggcgccggttcggtggcttgccaggagttcattacccagttgggccgtaacttccaggtgatctacgagcaggccaacgcccacgggcagaaggtgcaggctgccggcaacaacatggcgcaaaccgacagcgccgtcggctccagctgggccactagtatgagccttttggatgctcatatcccacagttggtggcctcccagtcggcgtttgccgccaaggcggggctgatgcggcacacgatcggtcaggccgagcaggcggcgatgtcggctcaggcgtttcaccagggggagtcgtcggcggcgtttcaggccgcccatgcccggtttgtggcggcggccgccaaagtcaacaccttgttggatgtcgcgcaggcgaatctgggtgaggccgccggtacctatgtggccgccgatgctgcggccgcgtcgacctataccgggttcgatatcatggatttcgggcttttacctccggaagtgaattcaagccgaatgtattccggtccggggccggagtcgatgctagccgccgcggccgcctgggacggtgtggccgcggagttgacttccgccgcggtctcgtatggatcggtggtgtcgacgctgatcgttgagccgtggatggggccggcggcggccgcgatggcggccgcggcaacgccgtatgtggggtggctggccgccacggcggcgctggcgaaggagacggccacacaggcgagggcagcggcggaagcgtttgggacggcgttcgcgatgacggtgccaccatccctcgtcgcggccaaccgcagccggttgatgtcgctggtcgcggcgaacattctggggcaaaacagtgcggcgatcgcggctacccaggccgagtatgccgaaatgtgggcccaagacgctgccgtgatgtacagctatgagggggcatctgcggccgcgtcggcgttgccgccgttcactccacccgtgcaaggcaccggcccggccgggcccgcggccgcagccgcggcgacccaagccgccggtgcgggcgccgttgcggatgcacaggcgacactggcccagctgcccccggggatcctgagcgacattctgtccgcattggccgccaacgctgatccgctgacatcgggactgttggggatcgcgtcgaccctcaacccgcaagtcggatccgctcagccgatagtgatccccaccccgataggggaattggacgtgatcgcgctctacattgcatccatcgcgaccggcagcattgcgctcgcgatcacgaacacggccagaccctggcacatcggcctatacgggaacgccggcgggctgggaccgacgcagggccatccactgagttcggcgaccgacgagccggagccgcactggggccccttcgggggcgcggcgccggtgtccgcgggcgtcggccacgcagcattagtcggagcgttgtcggtgccgcacagctggaccacggccgccccggagatccagctcgccgttcaggcaacacccaccttcagctccagcgccggcgccgacccgacggccctaaacgggatgccggcaggcctgctcagcgggatggctttggcgagcctggccgcacgcggcacgacgggcggtggcggcacccgtagcggcaccagcactgacggccaagaggacggccgcaaacccccggtagttgtgattagagagcagccgccgcccggaaaccccccgcggtaagatttctaaatccatcacactggcggccgctcgag

SEQ ID NO: 49 - protein, 710 aa, Artificial Sequence
  Description of Artificial Sequencetetra-fusion protein DPV-MTI-MSL-MTCC#2 (designated (MTb71F)
HMHHHHHHDPVDAVINTTCNYGQVVAALNATDPGAAAQFNASPVAQSYLRNFLAAPPPQRAAMAAQLQAVPGAAQYIGLVESVAGSCNNYELMTINYQFGDVDAHGAMIRAQAASLEAEHQAIVRDVLAAGDFWGGAGSVACQEFITQLGRNFQVIYEQANAHGQKVQAAGNNMAQTDSAVGSSWATSMSLLDAHIPQLVASQSAFAAKAGLMRHTIGQAEQAAMSAQAFHQGESSAAFQAAHARFVAAAAKVNTLLDVAQANLGEAAGTYVAADAAAASTYTGFDIMDFGLLPPEVNSSRMYSGPGPESMLAAAAAWDGVAAELTSAAVSYGSVVSTLIVEPWMGPAAAAMAAAATPYVGWLAATAALAKETATQARAAAEAFGTAFAMTVPPSLVAANRSRLMSLVAANILGQNSAAIAATQAEYAEMWAQDAAVMYSYEGASAAASALPPFTPPVQGTGPAGPAAAAAATQAAGAGAVADAQATLAQLPPGILSDILSALAANADPLTSGLLGIASTLNPQVGSAQPIVIPTPIGELDVIALYIASIATGSIALAITNTARPWHIGLYGNAGGLGPTQGHPLSSATDEPEPHWGPFGGAAPVSAGVGHAALVGALSVPHSWTTAAPEIQLAVQATPTFSSSAGADPTALNGMPAGLLSGMALASLAARGTTGGGGTRSGTSTDGQEDGRKPPVVVIREQPPPGNPPR

SEQ ID NO: 50 - DNA, 873 bp, Artificial Sequence
  Description of Artificial SequenceMAPS-DPVpET Leish-Tb fusion construct, Leishmania thiol-specific antioxidant (TBA or MAPS) linked with TB DPV (MTB8.4)
atgcatcaccatcaccatcacatgtcctgcggtaacgccaagatcaactctcccgcgccgtccttcgaggaggtggcgctcatgcccaacggcagcttcaagaagatcagcctctcctcctacaagggcaagtgggtcgtgctcttcttctacccgctcgacttcaccttcgtgtgcccgacagaggtcatcgcgttctccgacagcgtgagtcgcttcaacgagctcaactgcgaggtcctcgcgtgctcgatagacagcgagtacgcgcacctgcagtggacgctgcaggaccgcaagaagggcggcctcgggaccatggcgatcccaatgctagccgacaagaccaagagcatcgctcgttcctacggcgtgctggaggagagccagggcgtggcctaccgcggtctcttcatcatcgacccccatggcatgctgcgtcagatcaccgtcaatgacatgccggtgggccgcagcgtggaggaggttctacgcctgctggaggcttttcagttcgtggagaagcacggcgaggtgtgccccgcgaactggaagaagggcgcccccacgatgaagccggaaccgaatgcgtctgtcgagggatacttcagcaagcaggaattcgatcccgtggacgcggtcattaacaccacctgcaattacgggcaggtagtagctgcgctcaacgcgacggatccgggggctgccgcacagttcaacgcctcaccggtggcgcagtcctatttgcgcaatttcctcgccgcaccgccacctcagcgcgctgccatggccgcgcaattgcaagctgtgccgggggcggcacagtacatcggccttgtcgagtcggttgccggctcctgcaacaactattaa

SEQ ID NO: 51 - protein, 290 aa, Artificial Sequence
  Description of Artificial SequenceMAPS-DPVpET Leish-Tb fusion construct, Leishmania thiol-specific antioxidant (TBA or MAPS) linked with TB DPV (MTB8.4)
MHHHHHHMSCGNAKINSPAPSFEEVALMPNGSFKKISLSSYKGKWVVLFFYPLDFTFVCPTEVIAFSDSVSRFNELNCEVLACSIDSEYAHLQWTLQDRKKGGLGTMAIPMLADKTKSIARSYGVLEESQGVAYRGLFIIDPHGMLRQITVNDMPVGRSVEEVLRLLEAFQFVEKHGEVCPANWKKGAPTMKPEPNASVEGYFSKQEFDPVDAVINTTCNYGQVVAALNATDPGAAAQFNASPVAQSYLRNFLAAPPPQRAAMAAQLQAVPGAAQYIGLVESVAGSCNNY

SEQ ID NO: 52 - DNA, 987 bp, Artificial Sequence
  Description of Artificial SequenceMAPS-DPASpET Leish-Tb fusion construct, Leishmania thiol-specific antioxidant (TSA or MAPS) linked with TB DPAS (MTB12)
atgcatcaccatcaccatcacatgtcctgcggtaacgccaagatcaactctcccgcgccgtccttcgaggaggtggcgctcatgcccaacggcagcttcaagaagatcagcctctcctcctacaagggcaagtgggtcgtgctcttcttctacccgctcgacttcaccttcgtgtgcccgacagaggtcatcgcgttctccgacagcgtgagtcgcttcaacgagctcaactgcgaggtcctcgcgtgctcgatagacagcgagtacgcgcacctgcagtggacgctgcaggaccgcaagaagggcggcctcgggaccatggcgatcccaatgctagccgacaagaccaagagcatcgctcgttcctacggcgtgctggaggagagccagggcgtggcctaccgcggtctcttcatcatcgacccccatggcatgctgcgtcagatcaccgtcaatgacatgccggtgggccgcagcgtggaggaggttctacgcctgctggaggcttttcagttcgtggagaagcacggcgaggtgtgccccgcgaactggaagaagggcgcccccacgatgaagccggaaccgaatgcgtctgtcgagggatacttcagcaagcaggaattcgacccggcatccgcccctgacgtcccgaccgccgcccagttgaccagcctgctcaacagcctcgccgatcccaacgtgtcgtttgcgaacaagggcagtctggtcgagggcggcatcgggggcaccgaggcgcgcatcgccgaccacaagctgaagaaggccgccgagcacggggatctgccgctgtcgttcagcgtgacgaacatccagccggcggccgccggttcggccaccgccgacgtttccgtctcgggtccgaagctctcgtcgccggtcacgcagaacgtcacgttcgtgaatcaaggcggctggatgctgtcacgcgcatcggcgatggagttgctgcaggccgcagggaactaa

SEQ ID NO: 53 - protein, 328 aa, Artificial Sequence
  Description of Artificial SequenceMAPS-DPASpET Leish-Tb fusion construct, Leishmania thiol-specific antioxidant (TSA or MAPS) linked with TB DPAS (MTB12)
MHHHHHHMSCGNAKINSPAPSFEEVALMPNGSFKKISLSSYKGKWVVLFFYPLDFTFVCPTEVIAFSDSVSRFNELNCEVLACSIDSEYAHLQWTLQDRKKGGLGTMAIPMLADKTKSIARSYGVLEESQGVAYRGLFIIDPHGMLRQITVNDMPVGRSVEEVLRLLEAFQFVEKHGEVCPANWKKGAPTMKPEPNASVEGYFSKQEFDPASAPDVPTAAQLTSLLNSLADPNVSFANKGSLVEGGIGGTEARIADHKLKKAAEHGDLPLSFSVTNIQPAAAGSATADVSVSGPKLSSPVTQNVTFVNQGGWMLSRASAMELLQAAGN

SEQ ID NO: 54 - DNA, 852 bp, Artificial Sequence
  Description of Artificial SequenceMAPS-DPVpc fusion DNA vaccine construct, Leishmania thiol-specific antioxidant (TSA or MAPS) linked with TB DPV (MTB8.4)
atgtcctgcggtaacgccaagatcaactctcccgcgccgtccttcgaggaggtggcgctcatgcccaacggcagcttcaagaagatcagcctctcctcctacaagggcaagtgggtcgtgctcttcttctacccgctcgacttcaccttcgtgtgcccgacagaggtcatcgcgttctccgacagcgtgagtcgcttcaacgagctcaactgcgaggtcctcgcgtgctcgatagacagcgagtacgcgcacctgcagtggacgctgcaggaccgcaagaagggcggcctcgggaccatggcgatcccaatgctagccgacaagaccaagagcatcgctcgttcctacggcgtgctggaggagagccagggcgtggcctaccgcggtctcttcatcatcgacccccatggcatgctgcgtcagatcaccgtcaatgacatgccggtgggccgcagcgtggaggaggttctacgcctgctggaggcttttcagttcgtggagaagcacggcgaggtgtgccccgcgaactggaagaagggcgcccccacgatgaagccggaaccgaatgcgtctgtcgagggatacttcagcaagcaggaattcgatcccgtggacgcggtcattaacaccacctgcaattacgggcaggtagtagctgcgctcaacgcgacggatccgggggctgccgcacagttcaacgcctcaccggtggcgcagtcctatttgcgcaatttcctcgccgcaccgccacctcagcgcgctgccatggccgcgcaattgcaagctgtgccgggggcggcacagtacatcggccttgtcgagtcggttgccggctcctgcaacaactattaa

SEQ ID NO: 55 - DNA, 966 bp, Artificial Sequence
  Description of Artificial SequenceMAPS-DPASpc fusion DNA vaccine construct, Leishmania thiol-specific antioxidant (TSA or MAPS) linked with TB DPAS (MTB12)
atgtcctgcggtaacgccaagatcaactctcccgcgccgtccttcgaggaggtggcgctcatgcccaacggcagcttcaagaagatcagcctctcctcctacaagggcaagtgggtcgtgctcttcttctacccgctcgacttcaccttcgtgtgcccgacagaggtcatcgcgttctccgacagcgtgagtcgcttcaacgagctcaactgcgaggtcctcgcgtgctcgatagacagcgagtacgcgcacctgcagtggacgctgcaggaccgcaagaagggcggcctcgggaccatggcgatcccaatgctagccgacaagaccaagagcatcgctcgttcctacggcgtgctggaggagagccagggcgtggcctaccgcggtctcttcatcatcgacccccatggcatgctgcgtcagatcaccgtcaatgacatgccggtgggccgcagcgtggaggaggttctacgcctgctggaggcttttcagttcgtggagaagcacggcgaggtgtgccccgcgaactggaagaagggcgcccccacgatgaagccggaaccgaatgcgtctgtcgagggatacttcagcaagcaggaattcgacccggcatccgcccctgacgtcccgaccgccgcccagttgaccagcctgctcaacagcctcgccgatcccaacgtgtcgtttgcgaacaagggcagtctggtcgagggcggcatcgggggcaccgaggcgcgcatcgccgaccacaagctgaagaaggccgccgagcacggggatctgccgctgtcgttcagcgtgacgaacatccagccggcggccgccggttcggccaccgccgacgtttccgtctcgggtccgaagctctcgtcgccggtcacgcagaacgtcacgttcgtgaatcaaggcggctggatgctgtcacgcgcatcggcgatggagttgctgcaggccgcagggaactaa

SEQ ID NO: 56 - DNA, 864 bp, Artificial Sequence
  Description of Artificial SequenceMAPS-DPV-AC fusion construct, Leishmania thiol-specific antioxidant (TSA or MAPS) linked with TB DPV (MTB8.4) codon optimized for eukaryotic expression (altered codon)
ggatccatgtcctgcggtaacgccaagatcaactctcccgcgccgtccttcgaggaggtggcgctcatgcccaacggcagcttcaagaagatcagcctctcctcctacaagggcaagtgggtcgtgctcttcttctacccgctcgacttcaccttcgtgtgcccgacagaggtcatcgcgttctccgacagcgtgagtcgcttcaacgagctcaactgcgaggtcctcgcgtgctcgatagacagcgagtacgcgcacctgcagtggacgctgcaggaccgcaagaagggcggcctcgggaccatggcgatcccaatgctagccgacaagaccaagagcatcgctcgttcctacggcgtgctggaggagagccagggcgtggcctaccgcggtctcttcatcatcgacccccatggcatgctgcgtcagatcaccgtcaatgacatgccggtgggccgcagcgtggaggaggttctacgcctgctggaggcttttcagttcgtggagaagcacggcgaggtgtgccccgcgaactggaagaagggcgcccccacgatgaagccggaaccgaatgcgtctgtcgagggatacttcagcaagcaggaattcgaccccgtggacgccgtgatcaacaccacctgcaactacggccaggtggtggctgccctgaacgcgaccgaccccggcgctgccgcacagttcaacgcctcccctgtggcccagtcctacctgcgcaacttcctcgccgcacccccacctcagcgcgctgccatggccgcccagctgcaggctgtgcccggcgccgcacagtacatcggcctggtcgagtccgtggccggctcctgcaacaactactaagaattc

SEQ ID NO: 57 - protein, 283 aa, Artificial Sequence
  Description of Artificial SequenceMAPS-DPV-AC fusion construct, Leishmania thiol-specific antioxidant (TSA or MAPS) linked with TB DPV (MTB8.4) codon optimized for eukaryotic expression (altered codon)
MSCGNAKINSPAPSFEEVALMPNGSFKKISLSSYKGKWVVLFFYPLDFTFVCPTEVIAFSDSVSRFNELNCEVLACSIDSEYAHLQWTLQDRKKGGLGTMAIPMLADKTKSIARSYGVLEESQGVAYRGLFIIDPHGMLRQITVNDMPVGRSVEEVLRLLEAFQFVEKHGEVCPANWKKGAPTMKPEPNASVEGYFSKQEFDPVDAVINTTCNYGQVVAALNATDPGAAAQFNASPVAQSYLRNFLAAPPPQRAAMAAQLQAVPGAAQYIGLVESVAGSCNNY

SEQ ID NO: 58 - DNA, 978 bp, Artificial Sequence
  Description of Artificial SequenceMAPS-DPAS-AC fusion construct, Leishmania thiol-specific antioxidant (TSA or MAPS) linked with TB DPAS (MTB12) codon optimized for eukaryotic expression (altered codon)
ggatccatgtcctgcggtaacgccaagatcaactctcccgcgccgtccttcgaggaggtggcgctcatgcccaacggcagcttcaagaagatcagcctctcctcctacaagggcaagtgggtcgtgctcttcttctacccgctcgacttcaccttcgtgtgcccgacagaggtcatcgcgttctccgacagcgtgagtcgcttcaacgagctcaactgcgaggtcctcgcgtgctcgatagacagcgagtacgcgcacctgcagtggacgctgcaggaccgcaagaagggcggcctcgggaccatggcgatcccaatgctagccgacaagaccaagagcatcgctcgttcctacggcgtgctggaggagagccagggcgtggcctaccgcggtctcttcatcatcgacccccatggcatgctgcgtcagatcaccgtcaatgacatgccggtgggccgcagcgtggaggaggttctacgcctgctggaggcttttcagttcgtggagaagcacggcgaggtgtgccccgcgaactggaagaagggcgcccccacgatgaagccggaaccgaatgcgtctgtcgagggatacttcagcaagcaggaattcgaccccgcctccgcccctgacgtgcccaccgccgcccagctgaccagcctgctgaacagcctcgccgaccccaacgtgtccttcgccaacaagggcagcctggtggagggcggcatcgggggcaccgaggctcgcatcgccgaccacaagctgaagaaggccgccgagcacggggacctgcccctgtccttcagcgtgaccaacatccagcctgctgccgccggctccgccaccgccgacgtgtccgtctctggccctaagctctcctctcccgtcacccagaacgtgacattcgtgaaccagggcggctggatgctgtcccgcgcctccgctatggagctgctgcaggccgcagggaactgagaattc

SEQ ID NO: 59 - protein, 321 aa, Artificial Sequence
  Description of Artificial SequenceMAPS-DPAS-AC fusion construct, Leishmania thiol-specific antioxidant (TSA or MAPS) linked with TB DPAS (MTB12) codon optimized for eukaryotic expression (altered codon)
MSCGNAKINSPAPSFEEVALMPNGSFKKISLSSYKGKWVVLFFYPLDFTFVCPTEVIAFSDSVSRFNELNCEVLACSIDSEYAHLQWTLQDRKKGGLGTMAIPMLADKTKSIARSYGVLEESQGVAYRGLFIIDPHGMLRQITVNDMPVGRSVEEVLRLLEAFQFVEKHGEVCPANWKKGAPTMKPEPNASVEGYFSKQEFDPASAPDVPTAAQLTSLLNSLADPNVSFANKGSLVEGGIGGTEARIADHKLKKAAEHGDLPLSFSVTNIQPAAAGSATADVSVSGPKLSSPVTQNVTFVNQGGWMLSRASAMELLQAAGN

SEQ ID NO: 60 - DNA, 264 bp, Artificial Sequence
  Description of Artificial SequenceMAPS(N5)-DPV-AC fusion construct, first 5 amino acids of Leishmaniathiol-specific antioxidant (TSA or MAPS) linked with TB DPV (MTB8.4) codon optimized for eukaryotic expression (altered codon)
atgtcctgcggtaacgaccccgtggacgccgtgatcaacaccacctgcaactacggccaggtggtggctgccctgaacgcgaccgaccccggcgctgccgcacagttcaacgcctcccctgtggcccagtcctacctgcgcaacttcctcgccgcacccccacctcagcgcgctgccatggccgcccagctgcaggctgtgcccggcgccgcacagtacatcggcctggtcgagtccgtggccggctcctgcaacaactactaa

SEQ ID NO: 61 - protein, 87 aa, Artificial Sequence
  Description of Artificial SequenceMAPS(N5)-DPV-AC fusion construct, first 5 amino acids of Leishmaniathiol-specific antioxidant (TSA or MAPS) linked with TB DPV (MTB8.4) codon optimized for eukaryotic expression (altered codon)
MSCGNDPVDAVINTTCNYGQVVAALNATDPGAAAQFNASPVAQSYLRNFLAAPPPQRAAMAAQLQAVPGAAQYIGLVESVAGSCNNY

SEQ ID NO: 62 - DNA, 279 bp, Artificial Sequence
  Description of Artificial SequenceMAPS(N10)-DPV-AC fusion construct, first 10 amino acids of Leishmaniathiol-specific antioxidant (TSA or MAPS) linked with TB DPV (MTB8.4) codon optimized for eukaryotic expression (altered codon)
atgtcctgcggtaacgccaagatcaactctgaccccgtggacgccgtgatcaacaccacctgcaactacggccaggtggtggctgccctgaacgcgaccgaccccggcgctgccgcacagttcaacgcctcccctgtggcccagtcctacctgcgcaacttcctcgccgcacccccacctcagcgcgctgccatggccgcccagctgcaggctgtgcccggcgccgcacagtacatcggcctggtcgagtccgtggccggctcctgcaacaactactaa

SEQ ID NO: 63 - protein, 92 aa, Artificial Sequence
  Description of Artificial SequenceMAPS(N10)-DPV-AC fusion construct, first 10 amino acids of Leishmaniathiol-specific antioxidant (TSA or MAPS) linked with TB DPV (MTB8.4) codon optimized for eukaryotic expression (altered codon)
MSCGNAKINSDPVDAVINTTCNYGQVVAALNATDPGAAAQFNASPVAQSYLRNFLAAPPPQRAAMAAQLQAVPGAAQYIGLVESVAGSCNNY

SEQ ID NO: 64 - DNA, 2808 bp, Artificial Sequence
  Description of Artificial SequenceMTB72F-MAPS (r95f) fusion construct, TB MTB72F (Ra12-TbH9-Ra35) linked to Leishmania thiol-specific antioxidant (TSA or MAPS)
catatgcatcaccatcaccatcacacggccgcgtccgataacttccagctgtcccagggtgggcagggattcgccattccgatcgggcaggcgatggcgatcgcgggccagatccgatcgggtggggggtcacccaccgttcatatcgggcctaccgccttcctcggcttgggtgttgtcgacaacaacggcaacggcgcacgagtccaacgcgtggtcgggagcgctccggcggcaagtctcggcatctccaccggcgacgtgatcaccgcggtcgacggcgctccgatcaactcggccaccgcgatggcggacgcgcttaacgggcatcatcccggtgacgtcatctcggtgacctggcaaaccaagtcgggcggcacgcgtacagggaacgtgacattggccgagggacccccggccgaattcatggtggatttcggggcgttaccaccggagatcaactccgcgaggatgtacgccggcccgggttcggcctcgctggtggccgcggctcagatgtgggacagcgtggcgagtgacctgttttcggccgcgtcggcgtttcagtcggtggtctggggtctgacggtggggtcgtggataggttcgtcggcgggtctgatggtggcggcggcctcgccgtatgtggcgtggatgagcgtcaccgcggggcaggccgagctgaccgccgcccaggtccgggttgctgcggcggcctacgagacggcgtatgggctgacggtgcccccgccggtgatcgccgagaaccgtgctgaactgatgattctgatagcgaccaacctcttggggcaaaacaccccggcgatcgcggtcaacgaggccgaatacggcgagatgtgggcccaagacgccgccgcgatgtttggctacgccgcggcgacggcgacggcgacggcgacgttgctgccgttcgaggaggcgccggagatgaccagcgcgggtgggctcctcgagcaggccgccgcggtcgaggaggcctccgacaccgccgcggcgaaccagttgatgaacaatgtgccccaggcgctgcaacagctggcccagcccacgcagggcaccacgccttcttccaagctgggtggcctgtggaagacggtctcgccgcatcggtcgccgatcagcaacatggtgtcgatggccaacaaccacatgtcgatgaccaactcgggtgtgtcgatgaccaacaccttgagctcgatgttgaagggctttgctccggcggcggccgcccaggccgtgcaaaccgcggcgcaaaacggggtccgggcgatgagctcgctgggcagctcgctgggttcttcgggtctgggcggtggggtggccgccaacttgggtcgggcggcctcggtcggttcgttgtcggtgccgcaggcctgggccgcggccaaccaggcagtcaccccggcggcgcgggcgctgccgctgaccagcctgaccagcgccgcggaaagagggcccgggcagatgctgggcgggctgccggtggggcagatgggcgccagggccggtggtgggctcagtggtgtgctgcgtgttccgccgcgaccctatgtgatgccgcattctccggcagccggcgatatcgccccgccggccttgtcgcaggaccggttcgccgacttccccgcgctgcccctcgacccgtccgcgatggtcgcccaagtggggccacaggtggtcaacatcaacaccaaactgggctacaacaacgccgtgggcgccgggaccggcatcgtcatcgatcccaacggtgtcgtgctgaccaacaaccacgtgatcgcgggcgccaccgacatcaatgcgttcagcgtcggctccggccaaacctacggcgtcgatgtggtcgggtatgaccgcacccaggatgtcgcggtgctgcagctgcgcggtgccggtggcctgccgtcggcggcgatcggtggcggcgtcgcggttggtgagcccgtcgtcgcgatgggcaacagcggtgggcagggcggaacgccccgtgcggtgcctggcagggtggtcgcgctcggccaaaccgtgcaggcgtcggattcgctgaccggtgccgaagagacattgaacgggttgatccagttcgatgccgcgatccagcccggtgattcgggcgggcccgtcgtcaacggcctaggacaggtggtcggtatgaacacggccgcgtccggtaccatgtcctgcggtaacgccaagatcaactctcccgcgccgtccttcgaggaggtggcgctcatgcccaacggcagcttcaagaagatcagcctctcctcctacaagggcaagtgggtcgtgctcttcttctacccgctcgacttcaccttcgtgtgcccgacagaggtcatcgcgttctccgacagcgtgagtcgcttcaacgagctcaactgcgaggtcctcgcgtgctcgatagacagcgagtacgcgcacctgcagtggacgctgcaggaccgcaagaagggcggcctcgggaccatggcgatcccaatgctagccgacaagaccaagagcatcgctcgttcctacggcgtgctggaggagagccagggcgtggcctaccgcggtctcttcatcatcgacccccatggcatgctgcgtcagatcaccgtcaatgacatgccggtgggccgcagcgtggaggaggttctacgcctgctggaggcttttcagttcgtggagaagcacggcgaggtgtgccccgcgaactggaagaagggcgcccccacgatgaagccggaaccgaatgcgtctgtcgagggatacttcagcaagcagtaaggatccactagt

SEQ ID NO: 65 - protein, 930 aa, Artificial Sequence
  Description of Artificial SequenceMTB72F-MAPS (r95f) fusion construct, TB MTB72F (Ra12-TbH9-Ra35) linked to Leishmania thiol-specific antioxidant (TSA or MAPS)
MHHHHHHTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAEFMVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMSMTNSGVSMTNTLSSMLKGFAPAAAAQAVQTAAQNGVRAMSSLGSSLGSSGLGGGVAANLGRAASVGSLSVPQAWAAANQAVTPAARALPLTSLTSAAERGPGQMLGGLPVGQMGARAGGGLSGVLRVPPRPYVMPHSPAAGDIAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDSGGPVVNGLGQVVGMNTAASGTMSCGNAKINSPAPSFEEVALMPNGSFKKISLSSYKGKWVVLFFYPLDFTFVCPTEVIAFSDSVSRFNELNCEVLACSIDSEYAHLQWTLQDRKKGGLGTMAIPMLADKTKSIARSYGVLEESQGVAYRGLFIIDPHGMLRQITVNDMPVGRSVEEVLRLLEAFQFVEKHGEVCPANWKKGAPTMKPEPNASVEGYFSKQ

SEQ ID NO: 66 - DNA, 1691 bp, Leishmania major
  thiol-specific antioxidant (TSA or MAPS)
ctgtactttattgccaccagccagccatgtcctgcggtaacgccaagatcaactctcccgcgccgtccttcgaggaggtggcgctcatgcccaacggcagcttcaagaagatcagcctctcctcctacaagggcaagtgggtcgtgctcttcttctacccgctcgactttagcttcgtgtgcccgacagaggtcatcgcgttctccgacagcgtgagtcgcttcaacgagctcaactgcgaggtcctcgcgtgctcgatagacagcgagtacgcgcacctgcagtggacgctgcaggaccgcaagaagggcggcctcgggaccatggcgatcccaatgctagccgacaagaccaagagcatcgctcgttcctacggcgtgctggaggagagccagggcgtggcctaccgcggtctcttcatcatcgacccccatggcatgctgcgtcagatcaccgtcaatgacatgccggtgggccgcagcgtggaggaggttctacgcctgctggaggcttttcagttcgtggagaagcacggcgaggtgtgccccgcgaactggaagaagggcgcccccacgatgaagccggaaccgaatgcgtctgtcgaaggatacttcagcaagcagtaaacctgtgagcgtcgcaggagtcagtgtgacctcacccgcctctgccagtgggtgcgagagggcgtgagggattgtgggaaggctgttggatatgatgcagacagcgatgaatgcaactcccacacactggccctcctcagccctctccacacagacacacgcacgcatgtgcggtgcttctgctccttcatttcctgatgcggtttctttactatttatgttttcccctgttttggtttcgcggaatatggacgcacgaacgcagagagcagaggcgcgacacacacacacggggcgcacacgtgtaagcgacggagcgcgacacagaggggagaagccggaaaggatatatgaaatgaagatggtgtcccgcacacacaggcacgcacacggggcgaacgcgtggcgcgtcctttctatggtttcgctgctgctgttcgtgttctcgcttcgctggttgcagttccggctcgtgctgccctgcctgccactcttggtccgtgtctgtccgtgtccgtgtccgtgtgggggggggagagtttttcactcccccggtcacctctactgtttattatttatatgtttatttatttccatattgactcaattcattccgtgcgaaagccggcttgttccggcgtcttttgacgaacaactgccctatcagctggtgatggccgtgtagtggactgccatggcgttgacgggagcgggggattagggttcgattccggagagggagcctgagaaatagctaccacttctacggagggcagcaggcgcgcaaattgcccaatgtcaaaacaaaacgatgaggcagcgaaaagaaatagagttgtcagtccatttggattgtcatttcaatgggggatatttaaacccatccaatatcgagtaacaattggaggacaagtctggtgccagcacccgcggtaattccagctccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 67 - protein, 199 aa, Leishmania major
  thiol-specific antioxidant (TSA or MAPS)
MSCGNAKINSPAPSFEEVALMPNGSFKKISLSSYKGKWVVLFFYPLDFSFVCPTEVIAFSDSVSRFNELNCEVLACSIDSEYAHLQWTLQDRKKGGLGTMAIPMLADKTKSIARSYGVLEESQGVAYRGLFIIDPHGMLRQITVNDMPVGRSVEEVLRLLEAFQFVEKHGEVCPANWKKGAPTMKPEPNASVEGYFSKQ

SEQ ID NO: 68 - DNA, 1618 bp, Leishmania braziliensis
  LeIF (LbeIF4A) homolog to eIF4A eukaryotic ribosomal protein
ccactctctcggtcgtctgtctcccacgcgcgcacgcagttgatttccgccttcttaaacgctctctttttttttatttttcacctgaccaaccgcaccacgtcggcctccatcatgtcgcagcaagaccgagttgccccacaggaccaggactcgttcctcgacgaccagcccggcgtccgcccgatcccgtccttcgatgacatgccgttgcaccagaaccttctgcgcggcatctactcgtacggcttcgagaaaccgtccagcatccagcagcgcgccatcgcccccttcacgcgcggcggcgacatcatcgcgcaggcgcagtccggtaccggcaagacgggcgccttctccatcggcctgctgcagcgcctggacttccgccacaacctgatccagggcctcgtgctctccccgacccgcgagctggccctgcagacggcggaggtgatcagccgcatcggcgagttcctgtcgaacagcgcgaagttctgtgagacctttgtgggtggcacgcgcgtgcaggatgacctgcgcaagctgcaggctggcgtcgtcgtcgccgtggggacgccgggccgcgtgtccgacgtgatcaagcgcggcgcgctgcgcaccgagtccctgcgcgtgctggtgctcgacgaggctgatgagatgctgtctcagggcttcgcggatcagatttacgagatcttccgcttcctgccgaaggacatccaggtcgcgctcttctccgccacgatgccggaggaggtgctggagctgacaaagaagttcatgcgcgaccccgtacgcattctcgtgaagcgcgagagcctgacgctggagggcatcaagcagttcttcatcgccgtcgaggaggagcacaagctggacacgctgatggacctgtacgagaccgtgtccatcgcgcagtccgtcatcttcgccaacacccgccgcaaggtggactggatcgccgagaagctgaatcagagcaaccacaccgtcagcagcatgcacgccgagatgcccaagagcgaccgcgagcgcgtcatgaacaccttccgcagcggcagctcccgcgtgctcgtaacgaccgacctcgtggcccgcggcatcgacgtgcaccacgtgaacatcgtcatcaacttcgacctgccgacgaacaaggagaactacctgcaccgcattggccgcggcggccgctacggcgtaaagggtgttgccatcaacttcgtgacggagaaagacgtggagctgctgcacgagatcgaggggcactaccacacgcagatcgatgagctcccggtggactttgccgcctacctcggcgagtgagcgggcccctgccccccttccctgcccccctctcgcgacgagagaacgcacatcgtaacacagccacgcgaacgatagtaagggcgtgcggcggcgttcccctcctcctgccagcggcccccctccgcagcgcttctcttttgagaggggggcagggggaggcgctgcgcctggctggatgtgtgcttgagcttgcattccgtcaagcaagtgctttgttttaattatgcgcgccgttttgttgctcgtccctttcgttggtgttttttcggccgaaacggcgtttaaagca

SEQ ID NO: 69 - protein, 403 aa, Leishmania braziliensis
  LeIF (LbeIF4A) homolog to eIF4A eukaryotic ribosomal protein
MSQQDRVAPQDQDSFLDDQPGVRPIPSFDDMPLHQNLLRGIYSYGFEKPSSIQQRAIAPFTRGGDIIAQAQSGTGKTGAFSIGLLQRLDFRHNLIQGLVLSPTRELALQTAEVISRIGEFLSNSAKFCETFVGGTRVQDDLRKLQAGVVVAVGTPGRVSDVIKRGALRTESLRVLVLDEADEMLSQGFADQIYEIFRFLPKDIQVALFSATMPEEVLELTKKFMRDPVRILVKRESLTLEGIKQFFIAVEEEHKLDTLMDLYETVSIAQSVIFANTRRKVDWIAEKLNQSNHTVSSMHAEMPKSDRERVMNTFRSGSSRVLVTTDLVARGIDVHHVNIVINFDLPTNKENYLHRIGRGGRYGVKGVAINFVTEKDVELLHEIEGHYHTQIDELPVDFAAYLGE

SEQ ID NO: 70 - DNA, 3134 bp, Leishmania major
  M15 (stress-inducible 1 or LmSTI1)
caagtgtcgaaggacagtgttcnccgtgtgagatcgccggctgtgcgtgtgaaggcggtgccatcgganaaacaacaccggtgganccgcaggaaaccatctttctccgcaggtctctttttgttgtcgattgagagtgcnccaaaccctgctggtgcccttctcacatatcatgtttttcgttgtgcgctcgctttgcctttcctctcctttccctctcttccgtggtgccgtgtatacttctggcacccgctacgtcacttcgctggtttgaacagaaccactgtgaacacccacgggcgatcgcacacatacacatccctcactcacacacacagctacatctatcctacataaagctgaaaaaaaagtctacgaacaattttgtttttacagtgcgttgccgcacatttctccgtaatggacgcaactgagctgaagaacaaggggaacgaagagttctccgccggccgctatgtggaggcggtgaactacttctcaaaggcgatccagttggatgagcagaacagtgtcctctacagcaaccgctccgcctgttttgcagccatgcagaaatacaaggacgcgctggacgacgccgacaagtgcatctcgatcaagccgaattgggccaagggctacgtgcgccgaggagcagctctccatggcatgcgccgctacgacgatgccattgccgcgtatgaaaaggggctcaaggtggacccttccaacagcggctgcgcgcagggcgtgaaggacgtgcaggtagccaaggcccgcgaagcacgtgaccccatcgctcgcgtcttcaccccggaggcgttccgcaagatccaagagaatcccaagctgtctctacttatgctgcagccggactacgtgaagatggtagacaccgtcatccgcgacccttcgcagggccggctgtacatggaagaccagcgctttgccctgacgctcatgtacctgagcggaatgaagattcccaacgatggtgatggcgaggaggaggaacgtccgtctgcgaaggcggcagagacagcgaagccaaaagaggagaagcctctcaccgacaacgagaaggaggccctggcgctcaaggaggagggcaacaagctgtacctctcgaagaagtttgaggaggcgctgaccaagtaccaagaggcgcaggtgaaagaccccaacaacactttatacattctgaacgtgtcggccgtgtacttcgagcagggtgactacgacaagtgcatcgccgagtgcgagcacggtatcgagcacggtcgcgagaaccactgcgactacacaatcattgcgaagctcatgacccggaacgccttgtgcctccagaggcagaggaagtacgaggctgctatcgacctttacaagcgcgcccttgtcgagtggcgtaaccctgacaccctcaagaagctgacggagtgcgagaaggagcaccaaaaggcggtggaggaagcctacatcgatcctgagatcgcgaagcagaagaaagacgaaggtaaccagtacttcaaggaggataagttccccgaggccgtggcagcgtacacggaggccatcaagcgcaaccctgccgagcacacctcctacagcaatcgcgcggccgcgtacatcaagcttggagccttcaacgacgccctcaaggacgcggagaagtgcattgagctgaagcccgactttgttaagggctacgcgcgcaagggtcatgcttacttttggaccaagcagtacaaccgcgcgctgcaggcgtacgatgagggcctcaaggtggacccgagcaatgcggactgcaaggatgggcggtatcgcacaatcatgaagattcaggagatggcatctggccaatccgcggatggcgacgaggcggcgcgccgggccatggacgatcctgaaatcgcggcaatcatgcaagatagctacatgcaactagtgttgaaggagatgcagaacgatcccacgcgcattcaggagtacatgaaggactccgggatctcatcgaagatcaacaagctgatttcagctggcatcattcgttttggtcagtagacttctacgctgcctcatcttttccgtgtctttgcgtcggcgggtatcgtaaagcacaataaagcagcgattcacatgcacgagtaaagtgctgcgcctctcaaacacgacgtcgaggctgtggtgcagatgcgcgtcctgcatgaaggtagtgaagaggaaagtaagggatgttgtttgtgggccttcgtggctgcgcacacacctcttatctccttcgcttggtaccttctccctttttcgtcttcacccccctttctcttctcacgctctccctggcgcggtggtgcaacgatttcgttttatttacgtctgtgtagctcctctattcaacggtgcgatgacgctaacgaagctggcctgtattcggctaaggcgaaggcaaaagactaggaggggggggggaaggagacggcgtgaccatcactgcgaagaaacaagccgaagaaaaggccccgaacgcctgcatttccgcgcgccctcgcccgccttccttccttccttcgctctctctctctctctctctcgctatcttctcaacggagacatgaaaggcgtttgttaggaaaagaggggggggggaagagtgggacgacgcgctgcgtcttttgggcactggtcacgtgcgtcaccctctttttttatctctattggcactgtcttgtttcttttccctttcctatcatacgcgtctcgcaaacgactccgcgctgagcagccatgtgctgcggcgtggaggaagtacacagacatcacggatgcatatgtgcgcgtccgtgtacgcgcttgtatggggcttctaacagcgcctgtgtgtgtttgtgtgtgtgtgtgtgtgtgtgtctgtgtatttcgagcgtctgtatgctattctattaagcaccgaagaagagacacacacgacagcgaaggagatggtgtcggcttttcggctaatcactcccttccatagcttctctgaaggaggctctcttccagaggaatagactgcagatggggtccacgtttatctgaggagtcaacggaaaaaaaaaaaaaaaaaaaaaaaaaaaaaaaaaaaaaaaaaactcgag

SEQ ID NO: 71 - protein, 546 aa, Leishmania major
  M15 (stress-inducible 1 or LmSTI1)
MDATELKNKGNEEFSAGRYVEAVNYFSKAIQLDEQNSVLYSNRSACFAAMQKYKDALDDADKCISIKPNWAKGYVRRGAALHGMRRYDDAIAAYEKGLKVDPSNSGCAQGVKDVQVAKAREARDPIARVFTPEAFRKIQENPKLSLLMLQPDYVKMVDTVIRDPSQGRLYMEDQRFALTLMYLSGMKIPNDGDGEEEERPSAKAAETAKPKEEKPLTDNEKEALALKEEGNKLYLSKKFEEALTKYQEAQVKDPNNTLYILNVSAVYFEQGDYDKCIAECEHGIEHGRENHCDYTIIAKLMTRNALCLQRQRKYEAAIDLYKRALVEWRNPDTLKKLTECEKEHQKAVEEAYIDPEIAKQKKDEGNQYFKEDKFPEAVAAYTEAIKRNPAEHTSYSNRAAAYIKLGAFNDALKDAEKCIELKPDFVKGYARKGHAYFWTKQYNRALQAYDEGLKVDPSNADCKDGRYRTIMKIQEMASGQSADGDEAARRAMDDPEIAAIMQDSYMQLVLKEMQNDPTRIQEYMKDSGISSKINKLISAGIIRFGQ

SEQ ID NO: 72 - DNA, 2040 bp, Leishmania braziliensis
  6H homolog to eukaryotic 83-kDa heat shock protein (Lbhsp83)
cgcggtggcggccgctctagaactagtggatcccccgggctgcaggaattcggcacgagagagcctgacggacccggcggtgctgggcgaggagactcacctgcgcgtccgcgtggtgccggacaaggcgaacaagacgctgacggtggaggataacggcatcggcatgaccaaggcggacctcgtgaacaatctgggcacgatcgcgcgctccggcacgaaggctttcatggaggcactggaggccggcggcgacatgagcatgatcggccagttcggtgtcggcttctactccgcgtaccttgtggcggaccgcgtgacggtggtgtcgaagaacaactcggacgaggcgtactgggaatcgtctgcggggggcacgttcaccatcacgagcgtgcaggagtcggacatgaagcgcggcacgagtacaacgctgcacctaaaggaggaccagcaggagtacctggaggagcgccgggtgaaggagctgatcaagaagcactccgagttcatcggctacgacatcgagctgatggtggagaagacggcggagaaggaggtgacggacgaggacgaggaggaggacgagtcgaagaagaagtcctgcggggacgagggcgagccgaaggtggaggaggtgacggagggcggcgaggacaagaagaagaagacgaagaaggtgaaggaggtgaagaagacgtacgaggtcaagaacaagcacaagccgctctggacgcgcgacacgaaggacgtgacgaaggaggagtacgcggccttctacaaggccatctccaacgactgggaggacacggcggcgacgaagcacttctcggtggagggccagctggagttccgcgcgatcgcgttcgtgccgaagcgcgcgccgttcgacatgttcgagccgaacaagaagcgcaacaacatcaagctgtacgtgcgccgcgtgttcatcatggacaactgcgaggacctgtgcccggactggctcggcttcgtgaagggcgtcgtggacagcgaggacctgccgctgaacatctcgcgcgagaacctgcagcagaacaagatcctgaaggtgatccgcaagaacatcgtgaagaagtgcctggagctgttcgaagagatagcggagaacaaggaggactacaagcagttctacgagcagttcggcaagaacatcaagctgggcatccacgaggacacggcgaaccgcaagaagctgatggagttgctgcgcttctacagcaccgagtcgggggaggagatgacgacactgaaggactacgtgacgcgcatgaagccggagcagaagtcgatctactacatcactggcgacagcaagaagaagctggagtcgtcgccgttcatcgagaaggcgagacgctgcgggctcgaggtgctgttcatgacggagccgatcgacgagtacgtgatgcagcaggtgaaggacttcgaggacaagaagttcgcgtgcctgacgaaggaaggcgtgcacttcgaggagtccgaggaggagaagaagcagcgcgaggagaagaaggcggcgtgcgagaagctgtgcaagacgatgaaggaggtgctgggcgacaaggtggagaaggtgaccgtgtcggagcgcctgttgacgtcgccgtgcatcctggtgacgtcggagtttgggtggtcggcgcacatggaacagatcatgcgcaaccaggcgctgcgcgactccagcatggcgcagtacatggtgtccaagaagacgatggaggtgaaccccgaccaccccatcatcaaggagctgcgccgccgcgtggaggcggacgagaacgacaaggccgtgaaggacctcgtcttcctgctcttcgacacgtcgctgctcacgtccggcttccagctggatgaccccaccggctacgccgagcgcatcaaccgcatgatcaagctcggcctgtcgctcgacgaggaggaggaggaggtcgccgaggcgccgccggccgaggcagcccccgcggaggtcaccgccggcacctccagcatggagcaggtggactgagccggtaa

SEQ ID NO: 73 - protein, 656 aa, Leishmania braziliensis
  6H homolog to eukaryotic 83-kDa heat shock protein (Lbhsp83)
SLTDPAVLGEETHLRVRVVPDKANKTLTVEDNGIGMTKADLVNNLGTIARSGTKAFMEALEAGGDMSMIGQFGVGFYSAYLVADRVTVVSKNNSDEAYWESSAGGTFTITSVQESDMKRGTSTTLHLKEDQQEYLEERRVKELIKKHSEFIGYDIELMVEKTAEKEVTDEDEEEDESKKKSCGDEGEPKVEEVTEGGEDKKKKTKKVKEVKKTYEVKNKHKPLWTRDTKDVTKEEYAAFYKAISNDWEDTAATKHFSVEGQLEFRAIAFVPKRAPFDMFEPNKKRNNIKLYVRRVFIMDNCEDLCPDWLGFVKGVVDSEDLPLNISRENLQQNKILKVIRKNIVKKCLELFEEIAENKEDYKQFYEQFGKNIKLGIHEDTANRKKLMELLRFYSTESGEEMTTLKDYVTRMKPEQKSIYYITGDSKKKLESSPFIEKARRCGLEVLFMTEPIDEYVMQQVKDFEDKKFACLTKEGVHFEESEEEKKQREEKKAACEKLCKTMKEVLGDKVEKVTVSERLLTSPCILVTSEFGWSAHMEQIMRNQALRDSSMAQYMVSKKTMEVNPDHPIIKELRRRVEADENDKAVKDLVFLLFDTSLLTSGFQLDDPTGYAERINRMIKLGLSLDEEEEEVAEAPPAEAAPAEVTAGTSSMEQVD

SEQ ID NO: 74 - DNA, 392 bp, Artificial Sequence
  Description of Artificial SequenceDPAS-AC (MTB12) codon optimized for eukaryotic expression (altered codon)
gataaagcttgcaatcatggaccccgcctccgcccctgacgtgcccaccgccgcccagctgaccagcctgctgaacagcctcgccgaccccaacgtgtccttcgccaacaagggcagcctggtggagggcggcatcgggggcaccgaggctcgcatcgccgaccacaagctgaagaaggccgccgagcacggggacctgcccctgtccttcagcgtgaccaacatccagcctgctgccgccggctccgccaccgccgacgtgtccgtctctggccctaagctctcctctcccgtcacccagaacgtgacattcgtgaaccagggcggctggatgctgtcccgcgcctccgctatggagctgctgcaggccgcagggaactgagctagctatc

SEQ ID NO: 75 - DNA, 278 bp, Artificial Sequence
  Description of Artificial SequenceDPV-AC (MTB8.4) codon optimized for eukaryotic expression (altered codon)
gataaagcttgcaatcatggaccccgtggacgccgtgatcaacaccacctgcaactacggccaggtggtggctgccctgaacgcgaccgaccccggcgctgccgcacagttcaacgcctcccctgtggcccagtcctacctgcgcaacttcctcgccgcacccccacctcagcgcgctgccatggccgcccagctgcaggctgtgcccggcgccgcacagtacatcggcctggtcgagtccgtggccggctcctgcaacaactactaagctagctatc

SEQ ID NO: 76 - DNA, 849 bp, Artificial Sequence
  Description of Artificial SequenceDPV-AC/MAPS fusion protein with DPV-AC fused upstream of MAPS, TB DPV (MTB8.4) codon optimized for eukaryotic expression (altered codon) linked with Leishmania thiol-specific antioxidant (TSA or MAPS)
atggaccccgtggacgccgtgatcaacaccacctgcaactacggccaggtggtggctgccctgaacgcgaccgaccccggcgctgccgcacagttcaacgcctcccctgtggcccagtcctacctgcgcaacttcctcgccgcacccccacctcagcgcgctgccatggccgcccagctgcaggctgtgcccggcgccgcacagtacatcggcctggtcgagtccgtggccggctcctgcaacaactacatgtcctgcggtaacgccaagatcaactctcccgcgccgtccttcgaggaggtggcgctcatgcccaacggcagcttcaagaagatcagcctctcctcctacaagggcaagtgggtcgtgctcttcttctacccgctcgacttcaccttcgtgtgcccgacagaggtcatcgcgttctccgacagcgtgagtcgcttcaacgagctcaactgcgaggtcctcgcgtgctcgatagacagcgagtacgcgcacctgcagtggacgctgcaggaccgcaagaagggcggcctcgggaccatggcgatcccaatgctagccgacaagaccaagagcatcgctcgttcctacggcgtgctggaggagagccagggcgtggcctaccgcggtctcttcatcatcgacccccatggcatgctgcgtcagatcaccgtcaatgacatgccggtgggccgcagcgtggaggaggttctacgcctgctggaggcttttcagttcgtggagaagcacggcgaggtgtgccccgcgaactggaagaagggcgcccccacgatgaagccggaaccgaatgcgtctgtcgagggatacttcagcaagcagtag

SEQ ID NO: 77 - protein, 282 aa, Artificial Sequence
  Description of Artificial SequenceDPV-AC/MAPS fusion protein with DPV-AC fused upstream of MAPS, TB DPV (MTB8.4) codon optimized for eukaryotic expression (altered codon) linked with Leishmania thiol-specific antioxidant (TSA or MAPS)
MDPVDAVINTTCNYGQVVAALNATDPGAAAQFNASPVAQSYLRNFLAAPPPQRAAMAAQLQAVPGAAQYIGLVESVAGSCNNYMSCGNAKINSPAPSFEEVALMPNGSFKKISLSSYKGKWVVLFFYPLDFTFVCPTEVIAFSDSVSRFNELNCEVLACSIDSEYAHLQWTLQDRKKGGLGTMAIPMLADKTKSIARSYGVLEESQGVAYRGLFIIDPHGMLRQITVNDMPVGRSVEEVLRLLEAFQFVEKHGEVCPANWKKGAPTMKPEPNASVEGYFSKQ

SEQ ID NO: 78 - DNA, 51 bp, Artificial Sequence
  Description of Artificial Sequencemegaprimer PCR primer MAPSN5-DPV-AC
gataaagcttgcaatcatgtcctgcggtaacgaccccgtggacgccgtgat

SEQ ID NO: 79 - DNA, 66 bp, Artificial Sequence
  Description of Artificial Sequencemegaprimer PCR primer MAPSN10-DPV-AC
gataaagcttgcaatcatgtcctgcggtaacgccaagatcaactctgaccccgtggacgccgtgat

SEQ ID NO: 80 - DNA, 32 bp, Artificial Sequence
  Description of Artificial Sequencemegaprimer PCR primer DPV-AC-NheI-R
gatagctagcttagtagttgttgcaggagccg